(19) United States
(12) Patent Application Publication
(10) Pub. No.: US 2023/0272081 A1
(43) Pub. Date: Aug. 31, 2023

(54) SERUM HALF-LIFE EXTENDED PD-L1 INHIBITORY POLYPEPTIDES

(71) Applicant: Avacta Life Sciences Limited, Wetherby (GB)

(72) Inventors: Emma Jenkins, Stansted (GB); Estelle Adam, Hitchin (GB); Emma Stanley, Hitchin (GB); Amrik Basran, Cambridge (GB); Matthew P. Vincent, Amesbury, MA (US)

(73) Assignee: Avacta Life Sciences Limited, Wetherby (GB)

(21) Appl. No.: 18/018,218

(22) PCT Filed: Jul. 30, 2021
(86) PCT No.: PCT/EP2021/071417

§ 371 Date: Jan. 26, 2023

Related U.S. Application Data
(60) Provisional application No. 63/059,026, filed on Jul. 30, 2020.
(60) Provisional application No. 63/059,037, filed on Jul. 30, 2020.

(51) Int. Cl. C07K 16/28; C12N 15/86; A61P 35/00

(52) U.S. Cl. CPC C07K 16/2827; C12N 15/86; C07K 16/2809; A61P 35/00; C07K 2317/92; C07K 2319/00

(57) ABSTRACT

Provided herein, in some embodiments, are fusion proteins comprising recombinantly engineered variant of stefin polypeptide (an AFFIMER® polypeptide) that binds to PD-L1 and a polypeptide that binds to human serum albumin (HSA). Also provided herein, in some embodiments, are compositions containing the fusion proteins, methods of using the fusion proteins, and methods of producing the fusion proteins.

### RELATED APPLICATIONS

This application claims the benefit under 35 U.S.C. § 119(e) of U.S. 63/059,026, filed Jul. 30, 2020 and U.S. 63/059,037, filed Jul. 30, 2020, which are incorporated by reference herein in their entireties.

### BACKGROUND

Specific antibodies to programmed death-ligand 1 (PD-L1) have been developed as anti-cancer agents (see, e.g., U.S. Pat. Nos. 9,212,224 and 8,008,449). There exists a need however, for additional PD-L1 inhibitory activities useful in the treatment of cancer, infectious disease, and neurodegenerative disease.

### SUMMARY

Provided herein, in some aspects, are therapeutic proteins comprising an (at least one) recombinantly engineered variant of stefin polypeptide (AFFIMER® polypeptide) sequence that binds to PD-L1 and an (at least one) AFFIMER® polypeptide sequence that binds to serum albumin (such as human serum albumin or “HSA”). The PD-L1 and HSA AFFIMER® polypeptide sequences included in the “chimeric” protein can be linked covalently (such as by chemical cross-linking or as a fusion protein), or non-covalently associated (such as through multimerization domains or small molecule binding domains). Even in dimeric forms that would otherwise be below the renal filtration threshold size, these polypeptides have been shown in in vivo pharmacokinetic (PK) studies to have a serum half-life of at least 7 days and can be made, for example, in bacterial cells (e.g., Escherichia coli).

Some aspects of the present disclosure provide a chimeric protein, preferably a fusion protein, comprising an HSA binding recombinantly engineered variant of stefin polypeptide (AFFIMER® polypeptide) and a PD-L1 binding AFFIMER® polypeptide, wherein the HSA binding AFFIMER® polypeptide binds to HSA with a K_{d} of 1×10⁻⁶M or less at pH 6.0 and optionally a K_{d} for binding HSA at pH 7.4 that is at least half a log greater than the K_{d} for binding at pH 6.0, and wherein the PD-L1 binding AFFIMER® polypeptide binds to PD-L1 with a K_{d} of 1×10⁻⁶M or less.

Other aspects of the present disclosure provide a chimeric protein, preferably a fusion protein, comprising an HSA binding AFFIMER® polypeptide that binds to HSA and a PD-L1 binding AFFIMER® polypeptide that binds to PD-L1, wherein the protein has a circulating half-life in human subjects of greater than 10 hours, greater than 24 hours, greater than 48 hours, greater than 72 hours, greater than 96 hours, greater than 120 hours, greater than 144 hours, greater than 168 hours, greater than 192 hours, greater than 216 hours, greater than 240 hours, greater than 264 hours, greater than 288 hours, greater than 312 hours, greater than 336 hours or, greater than 360 hours.

Other aspects of the present disclosure provide a chimeric protein, preferably a fusion protein, comprising an HSA binding AFFIMER® polypeptide that binds to HSA and a PD-L1 binding AFFIMER® polypeptide that binds to PD-L1, wherein the protein has a circulating half-life in human subjects of at least 7 days, more preferably at least 10, 12, 14, 16, 18, 20, 22 or even 24 days.

In some embodiments, the polypeptides have a serum half-life in human patients of greater than 50%, greater than 60%, greater than 70%, or greater than 80% of the serum half-life of HSA.

In some embodiments, AFFIMER® polypeptides (HSA or PD-L1 binding AFFIMER® polypeptides) have amino acid sequences which are, each independently, represented in general formula (I):

FR1-(Xaa)ₙ-FR2-(Xaa)ₘ-FR3 (I)

wherein
FR1 is an amino acid sequence having at least 70% identity to MIPGGLSEAK PATPEIQEIV DKVKPQLEEK TNETYGKLEA VQYKTQVLA (SEQ ID NO: 1) or MIPRGLSEAK PATPEIQEIV DKVKPQLEEK TNETYGKLEA VQYKTQVLA (SEQ ID NO: 2);
FR2 is an amino acid sequence having at least 70% identity to GTNYYIKVRA GDNKYMHLKV FKSL (SEQ ID NO: 3) or STNYYIKVRA GDNKYMHLKV FNGP (SEQ ID NO: 4);
FR3 is an amino acid sequence having at least 70% identity to ADRVLTGYQV DKNKDDELTG F (SEQ ID NO: 5) or EDLVLTGYQV DKNKDDELTG F (SEQ ID NO: 6);
Xaa, individually for each occurrence, is an amino acid,
n is an integer from 3 to 20, and
m is an integer from 3 to 20.

In some embodiments, FR1 is a polypeptide sequence having at least 80%, 85%, 90%, 95% or even 98% homology with SEQ ID NO: 1 and/or 2. In some embodiments, FR1 is a polypeptide sequence having at least 80%, 85%, 90%, 95% or even 98% identity with SEQ ID NO: 1 and/or 2; In some embodiments, FR2 is a polypeptide sequence having at least 80%, 85%, 90%, 95% or even 98% homology with SEQ ID NO: 3 and/or 4. In some embodiments, FR2 is a polypeptide sequence having at least 80%, 85%, 90%, 95% or even 98% identity with SEQ ID NO: 3 and/or 4; In some embodiments, FR3 is a polypeptide sequence having at least 80%, 85%, 90%, 95% or even 98% homology with SEQ ID NO: 5 and/or 6. In some embodiments, FR3 is a polypeptide sequence having at least 80%, 85%, 90%, 95% or even 98% identity with SEQ ID NO: 5 and/or 6.

In some embodiments, the anti-PD-L1 AFFIMER® polypeptide has an amino acid sequence represented in the general formula:

(SEQ ID NO: 7)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF

wherein
Xaa, individually for each occurrence, is an amino acid residue; n and m are each, independently, an integer from 3 to 20; Xaa1 is Gly, Ala, Val, Arg, Lys, Asp, or Glu, more preferably Gly, Ala, Arg or Lys, and more even more preferably Gly or Arg; Xaa2 is Gly, Ala, Val, Ser or Thr, more preferably Gly or Ser; Xaa3 is Arg, Lys, Asn, Gln, Ser, Thr, more preferably Arg, Lys, Asn or Gln, and even more preferably Lys or Asn; Xaa4 is Gly, Ala, Val, Ser or Thr, more preferably Gly or Ser; Xaa5 is Ala, Val, Ile, Leu, Gly or Pro, more preferably Ile, Leu or Pro, and even more preferably Leu or Pro; Xaa6 is Gly, Ala, Val, Asp or Glu, more preferably Ala, Val, Asp or Glu, and even more preferably Ala or Glu; and Xaa7 is Ala, Val, Ile, Leu, Arg or Lys, more preferably Ile, Leu or Arg, and even more preferably Leu or Arg.

For instance, the anti-PD-L1 AFFIMER® polypeptide can have an amino acid sequence represented in the general formula:

(SEQ ID NO: 8)
MIPRGLSEAKPATPEIQEIVDKVKPQLEEKTNETYGKLEAVQYKTQVLA-
(Xaa)ₙ-STNYYIKVRAGDNKYMHLKVFNGP-(Xaa)ₘ-ADRVLTGYQVDK
NKDDELTGF,

wherein Xaa, individually for each occurrence, is an amino acid residue; n and m are each, independently, an integer from 3 to 20.

In some embodiments, n is 3 to 15, 3 to 12, 3 to 9, 3 to 7, 5 to 7, 5 to 9, 5 to 12, 5 to 15, 7 to 12 or 7 to 9.

In some embodiments, m is 3 to 15, 3 to 12, 3 to 9, 3 to 7, 5 to 7, 5 to 9, 5 to 12, 5 to 15, 7 to 12 or 7 to 9.

In some embodiments, Xaa, independently for each occurrence, is an amino acid that can be added to a polypeptide by recombinant expression in a prokaryotic or eukaryotic cell, and even more preferably one of the 20 naturally occurring amino acids.

In some embodiments of the above sequences and formulas, (Xaa)ₙ is an amino acid sequence represented in the general formula:

(SEQ ID NO: 308)
-aa1-aa2-aa3-Gly-Pro-aa4-aa5-Trp-aa6-

wherein
aa1 represents an amino acid residue with a basic sidechain, more preferably Lys, Arg or His, and even more preferably Lys or Arg;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain, even more preferably Ala, Pro, Ile, Gln, Thr, Asp, Glu, Lys, Arg or His, and even more preferably Ala, Gln, Asp or Glu;
aa3 represents an amino acid residue with an aromatic or basic sidechain, preferably Phe, Tyr, Trp, Lys, Arg or His, more preferably Phe, Tyr, Trp, and even more preferably His or Tyr, Trp or His;
aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu;
aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg; and
aa6 represents an amino acid residue with an aromatic or acid sidechain, preferably Phe, Tyr, Trp, Asp or Glu; more preferably Trp or Asp; and even more preferably Trp.

In some embodiments of the above sequences and formulas, (Xaa)ₙ is an amino acid sequence represented in the general formula:

(SEQ ID NO: 310)
-aa1-aa2-aa3-Phe-Pro-aa4-aa5-Phe-Trp-

wherein
aa1 represents an amino acid residue with a basic sidechain or aromatic sidechain, preferably Lys, Arg, His, Ser, Thr, Asn or Gln, more preferably Lys, Arg, His, Asn or Gln, and even more preferably Lys or Asn;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain, even more preferably Ala, Pro, Ile, Gln, Thr, Asp, Glu, Lys, Arg or His, and even more preferably Ala, Gln, Asp or Glu;
aa3 represents an amino acid residue with an aromatic or basic sidechain, preferably Phe, Tyr, Trp, Lys, Arg or His, more preferably Phe, Tyr, Trp or His, and even more preferably Tyr, Trp or His;
aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu; and
aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg.

In some embodiments of the above sequences and formulas, (Xaa)ₙ is an amino acid sequence selected from SEQ ID NOs: 16-50, or an amino acid sequence having at least 80%, 85%, 90%, 95% or even 98% homology with a sequence selected from SEQ ID NOs: 16-50.

In some embodiments of the above sequences and formulas, (Xaa)ₘ is an amino acid sequence represented in the general formula:

(SEQ ID NO: 311)
-aa7-aa8-aa9-aa10-aa11-aa12-aa13-aa14-aa15-

wherein
aa7 represents an amino acid residue with neutral polar or non-polar sidechain or an acidic sidechain; preferably Gly, Ala, Val, Pro, Trp, Gln, Ser, Asp or Glu, and even more preferably Gly, Ala, Trp, Gln, Ser, Asp or Glu;
aa8 represents an amino acid residue, preferably an amino acid residue with a neutral polar or non-polar sidechain or a charged (acidic or basic) sidechain or aromatic sidechain, more preferably a charged (acidic or basic) sidechain, more preferably Asp, Glu, Lys, Arg, His, Gln, Ser, Thr, Asn, Ala, Val, Pro, Gly, Tyr or Phe, and even more preferably Asp, Glu, Lys, Arg, His or Gln;
aa9 represents an amino acid residue, preferably an amino acid residue with a neutral polar or non-polar sidechain or a charged (acidic or basic) sidechain or aromatic sidechain, more preferably a neutral polar side chain or an acid side chain, more preferably Gln, Ser, Thr, Asn, Asp, Glu, Arg, Lys, Gly, Leu, Pro or Tyr, and even more preferably Gln, Thr or Asp;
aa10 represents an amino acid residue, preferably an amino acid residue with a neutral polar or non-polar sidechain or a charged (acidic or basic) sidechain or aromatic sidechain, more preferably a neutral polar side chain or a basic or acid side chain, more preferably Asp, Glu, Arg, His, Lys, Ser, Gln, Asn, Ala, Leu, Tyr, Trp, Pro or Gly, and even more preferably Asp, Glu, His, Gln, Asn, Leu, Trp or Gly;
aa11 represents an amino acid residue, preferably an amino acid residue with a neutral polar sidechain or a charged (acidic or basic) sidechain or a nonpolar aliphatic sidechain or an aromatic sidechain, more preferably a neutral polar side chain or a basic or acid side chain, more preferably Asp, Glu, Ser, Thr, Gln, Arg, Lys, His, Val, Ile, Tyr or Gly and even more preferably Asp, Glu, Ser, Thr, Gln, Lys or His;
aa12 represents an amino acid residue, preferably an amino acid residue with a neutral polar sidechain or a charged (acidic or basic) sidechain or a nonpolar aliphatic sidechain or an aromatic sidechain, more preferably an acid side chain, more preferably Asp, Glu, Ser, Thr, Gln, Asn, Lys, Arg, Val, Leu, Ile, Trp, Tyr, Phe or Gly and even more preferably Asp, Glu, Ser, Tyr, Trp, Arg or Lys;
aa13 represents an amino acid residue, preferably an amino acid residue with a neutral polar sidechain or a charged (acidic or basic) sidechain or a nonpolar aliphatic sidechain or an aromatic sidechain, more preferably an acid side chain, more preferably Ser, Thr, Gln, Asn, Val, Ile, Leu, Gly, Pro, Asp, Glu, His, Arg, Trp, Tyr or Phe and even more preferably Ser, Thr, Gln, Asn, Val, Ile, Leu, Gly, Asp or Glu;
aa14 represents an amino acid residue, preferably an amino acid residue with a neutral polar sidechain or a charged (acidic or basic) sidechain, more preferably Ala, Ile, Trp, Pro, Asp, Glu, Arg, Lys, His, Ser, Thr, Gln or Asn and even more preferably Ala, Pro, Asp, Glu, Arg, Lys, Ser, Gln or Asn; and
aa15 represents an amino acid residue, preferably an amino acid residue with a neutral polar or neutral non-polar sidechain or a charged (acidic or basic) sidechain, more preferably His, Arg, Lys, Asp, Ser, Thr, Gln, Asn, Ala, Val, Leu, Gly or Phe and even more preferably His, Arg, Lys, Asp, Ser, Thr, Gln or Asn.

In some embodiments of the above sequences and formulas, (Xaa)ₘ is an amino acid sequence selected from SEQ ID NOs: 51-85, or an amino acid sequence having at least 80%, 85%, 90%, 95% or even 98% homology with a sequence selected from SEQ ID NOs: 51-85.

In some embodiments, the PD-L1 binding AFFIMER® amino acid sequence has at least 70%, at least 75%, at least 80%, at least 85%, at least 90%, or at least 95% identity to an amino acid sequence of any one of SEQ ID NOS: 192-200. In some embodiments, the PD-L1 binding AFFIMER® amino acid sequence comprises an amino acid sequence of any one of SEQ ID NOS: 192-200.

For further illustration, the HSA binding AFFIMER® polypeptide(s) of the fusion protein can have an amino acid sequence represented in the general formula:

(SEQ ID NO: 9)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF,

wherein Xaa, individually for each occurrence, is an amino acid; n is an integer from 3 to 20, and m is an integer from 3 to 20; Xaa1 is Gly, Ala, Val, Arg, Lys, Asp, or Glu; Xaa2 is Gly, Ala, Val, Ser or Thr; Xaa3 is Arg, Lys, Asn, Gln, Ser, Thr; Xaa4 is Gly, Ala, Val, Ser or Thr; Xaa5 is Ala, Val, Ile, Leu, Gly or Pro; Xaa6 is Gly, Ala, Val, Asp or Glu; and Xaa7 is Ala, Val, Ile, Leu, Arg or Lys.

In some embodiments, the amino acid sequence of HSA binding AFFIMER® polypeptide provided herein is represented in general formula:

(SEQ ID NO: 10)
MIPRGLSEAKPATPEIQEIVDKVKPQLEEKTNETYGKLEAVQYKTQVLA-
(Xaa)ₙ-GTNYYIKVRAGDNKYMHLKVFKSL-(Xaa)m-EDL VLTGYQV
DKNKDDELTGF,

wherein Xaa, individually for each occurrence, is an amino acid; n is an integer from 3 to 20, and m is an integer from 3 to 20.

In some embodiments, (Xaa)ₙ is represented by formula:

-aa1-aa2-aa3-aa4-aa5-aa6-aa7-aa8-aa9-

wherein aa1 is an amino acid with a neutral polar hydrophilic side chain; aa2 is an amino acid with a neutral nonpolar hydrophobic side chain; aa3 is an amino acid with a neutral nonpolar hydrophobic side chain; aa4 is an amino acid with a neutral polar hydrophilic side chain; aa5 is an amino acid with a positively charged polar hydrophilic side chain; aa6 is an amino acid with a positively charged polar hydrophilic side chain; aa7 is an amino acid with a neutral nonpolar hydrophobic side chain; aa8 is an amino acid with a neutral nonpolar hydrophobic side chain; and aa9 is an amino acid with a neutral nonpolar hydrophilic side chain.

In some embodiments, (Xaa)ₘ is represented by formula:

-aa1-aa2-aa3-aa4-aa5-aa6-aa7-aa8-aa9-

wherein aa1 is an amino acid with a neutral nonpolar hydrophobic side chain; aa2 is an amino acid with a positively charged polar hydrophilic side chain; aa3 is an amino acid with a neutral nonpolar hydrophobic side chain; aa4 is an amino acid with a positively charged polar hydrophilic side chain; aa5 is an amino acid with a neutral polar hydrophilic side chain; aa6 is an amino acid with a neutral polar hydrophilic side chain; aa7 is an amino acid with a negatively charged polar hydrophilic side chain; aa8 is an amino acid with a positively charged polar hydrophilic side chain; and aa9 is an amino acid with a neutral nonpolar hydrophilic side chain.

In some embodiments, the amino acid with the neutral nonpolar hydrophilic side chain is selected from cysteine (C or Cys) and glycine (G or Gly); the amino acid with the neutral nonpolar hydrophobic side chain is selected from alanine (A or Ala), isoleucine (I or Ile), leucine (L or Leu), methionine (M or Met), phenylalanine (F or Phe), proline (P or Pro), tryptophan (W or Trp), and valine (V or Val); the amino acid with the neutral polar hydrophilic side chain is selected from asparagine (N or Asn), glutamine (Q or Gln), serine (S or Ser), threonine (T or Thr), and tyrosine (Y or Tyr); the amino acid with the positively charged polar hydrophilic side chain is selected from arginine (R or Arg), histidine (H or His), and lysine (K or Lys); and the amino acid with the negatively charged polar hydrophilic side chain is selected from aspartate (D or Asp) and glutamate (E or Glu).

In some embodiments, (Xaa)ₙ is represented by formula:

(SEQ ID NO: 309)
-aa1-aa2-aa3-aa4-aa5-aa6-aa7-aa8-aa9-

wherein aa1 is an amino acid selected from D, G, N, and V; aa2 is an amino acid selected from W, Y, H, and F; aa3 is an amino acid selected from W, Y, G, W, and F; aa4 is an amino acid selected from Q, A, and P; aa5 is an amino acid selected from A, Q, E, R, and S; aa6 is an amino acid selected from K, R, and Y; aa7 is an amino acid selected from W and Q; aa8 is an amino acid selected from P and H; aa9 is an amino acid selected from H, G, and Q.

In some embodiments, (Xaa)ₙ is an amino acid sequence having at least 80% or at least 90% identity to the amino acid sequence of any one of SEQ ID NOs: 86-138. In some embodiments, (Xaa)ₙ is the amino acid sequence of any one of SEQ ID NOs: 86-138.

In some embodiments, (Xaa)ₘ is represented by formula:

(SEQ ID NO: 312)
-aa1-aa2-aa3-aa4-aa5-aa6-aa7-aa8-aa9-

wherein aa1 is an amino acid selected from Y, F, W, and N; aa2 is an amino acid selected from K, P, H, A, and T; aa3 is an amino acid selected from V, N, G, Q, A, and F; aa4 is an amino acid selected from H, T, Y, W, K, V, and R; aa5 is an amino acid selected from Q, S, G, P, and N; aa6 is an amino acid selected from S, Y, E, L, K, and T; aa7 is an amino acid selected from S, D, V, and K; aa8 is an amino acid selected from G, L, S, P, H, D, and R; aa9 is an amino acid selected from G, Q, E, and A.

In some embodiments, (Xaa)ₘ is an amino acid sequence having at least 80% or at least 90% identity to the amino acid sequence of any one of SEQ ID NOs: 139-191. In some embodiments, (Xaa)ₘ is the amino acid sequence of any one of SEQ ID NOs: 139-191.

In some embodiments, the HSA binding AFFIMER® amino acid sequence has at least 70%, at least 75%, at least 80%, at least 85%, at least 90%, or at least 95% identity to an amino acid sequence of any one of SEQ ID NOs: 201-235. In some embodiments, the HSA binding AFFIMER® amino acid sequence comprises an amino acid sequence of any one of SEQ ID NOs: 201-235.

In some embodiments, HSA binding AFFIMER® polypeptide has an amino acid sequence that can be encoded by a polynucleotide having a coding sequence that hybridizes to any one of SEQ ID NOs: 201-235 under stringent conditions of 6× sodium chloride/sodium citrate (SSC) at 45° C. followed by a wash in 0.2×SSC at 65° C., and wherein the PD-L1 binding AFFIMER® polypeptide has an amino acid sequence that can be encoded by a polynucleotide having a coding sequence that hybridizes to any one of SEQ ID NOS: 192-200 under stringent conditions of 6× sodium chloride/sodium citrate (SSC) at 45° C. followed by a wash in 0.2×SSC at 65° C.

In some embodiments, the HSA binding AFFIMER® polypeptide binds to HSA and/or the PD-L1 binding AFFIMER® polypeptide binds to PDL1 with a Kd of 1×10⁻⁷ M, a Kd of 1×10⁻⁸ M, or Kd of 1×10⁻⁹ M.

In some embodiments, the HSA binding AFFIMER® polypeptide binds to HSA at pH 7.4 with a K_{d} that is at least one log greater than the K_{d} for binding to HSA at pH 6.0, at least 1.5 logs greater than the K_{d} for binding to HSA at pH 6, at least 2 logs greater than the K_{d} for binding to HSA at pH 6, or at least 2.5 log greater than the K_{d} for binding to HSA at pH 6.

In some embodiments, the chimeric protein does not inhibit binding of human serum albumin to HSA.

In some embodiments, the protein does not inhibit binding of IgG to HSA.

In some embodiments, the therapeutic proteins of the present disclosure include, in addition to at least on PD-L1 binding AFFIMER® sequence and at least one HSA binding AFFIMER® sequence, the sequence(s) of one or more additional polypeptides which may confer additional therapeutic activity and/or which may confer additional PK/ADME properties to the resulting therapeutic protein.

In some embodiments, the additional polypeptide ligand can be an immunostimulatory cytokine that promotes antitumor immunity, such as IFN-α, IL-2, IL-15, IL-21, and IL-12, or a variant sequence thereof.

For example, the additional polypeptide ligand can be an IL-2 cytokine or a variant polypeptide sequence thereof. The IL-2 cytokine displays multiple immunological effects and acts by binding to the IL-2 receptor (IL-2R). The association of IL-2Rα (CD25), IL-2Rβ (CD122), and IL-2Rγ (CD132) subunits results in the trimeric high affinity IL-2Rαβγ. CD25 confers high affinity binding to IL-2, whereas the β and γ subunits (expressed on natural killer (NK) cells, monocytes, macrophages and resting CD4⁺ and CD8⁺ T cells) mediate signal transduction. It appears that the expression of CD25 is essential for the expansion of immunosuppressive regulatory T cells (Treg); on the other hand, cytolytic CD8⁺ T and NK cells can proliferate and kill target cells responding to IL-2 by the IL-2Rβγ engagement in the absence of CD25. In some embodiments including an IL-2 sequence as part of the fusion protein, the IL-2 polypeptide sequence is a mutant IL-2 polypeptide that comprises the amino acid sequence of SEQ ID NO: 11 having one or more amino acid substitutions that abolish or reduce affinity of the mutant IL-2 polypeptide (including in the context of the fusion protein) to the high-affinity IL-2 receptor (CD25) while preserving all or a substantial portion of the native affinity to the intermediate-affinity IL-2 receptor(s) as compared to a wild-type IL-2 polypeptide.

SEQ ID NO. 11
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFAQSIIS TLT

There have been a variety of published approaches that can attain suitable reductions in CD25 binding, including through multiple mutations to the IL-2 sequence. For instance, mutation of two amino acids, R38 and F42, were identified to significantly reduce binding to CD25, i.e., in context of the high affinity IL2R. Merely to illustrate, variant IL-2 polypeptide sequences that can be used include variant sequences to SEQ ID NO: 11 in which one or more of the following residues have been altered T3 (such as T3A), D20 (such as D20T), R38 (such as R38A and R38D), F42 (such as F42A, F42G, F42S, F42T, F42Q, F42E, F42N, F42D, F42R or F42K), K43 (such as K43E), E61 (such as E61R), E62 (such as E62A), Y45 (such as Y45A, Y45G, Y45S, Y45T, Y45Q, Y45E, Y45N, Y45D, Y45R or Y45K) and/or L72 (such as L72G, L72A, L72S, L72T, L72Q, L72E, L72N, L72D, L72R or L72K) as examples. In some embodiments, the IL-2 variant can include a combination of changes, including the set of R38D, K43E, E61R which targets charged residues, or R38A, F42A, Y45A, and E62A targeting both charged and aromatic residues. Exemplary variant IL-2 sequences that can be utilized as part of the subject fusion protein are also described in, for example, U.S. Pat. No. 9,266,938 and US Application 20060251617; Ghasemi et al. Nat Commun. (2016) 7: 12878; Tang et al. Cytokine: X (2019) 1(1): 1-9; and Heaton et al. Cancer Res June (1993) 53(11):2597-602, each of which is incorporated by reference herein.

The polypeptide sequences for exemplary PD-L1/XT/IL-2 variant fusion include the following, where the first underlined sequence is a secretion signal sequence and the second underlined sequence is a (G₄S)ₙ linked IL-2 variant polypeptide sequence:

SEQ ID NO: 12
MPLLLLLPLL WAGALAIPRG LSEAKPATPE IQEIVDKVKP
QLEEKTNETY GKLEAVQYKT QVLAREGRQD WVLSTNYYIK
VRAGDNKYMH LKVFNGPWVP FPHQQLADRV LTGYQVDKNK
DDELTGFAEA AAKEAAAKEA AAKEAAAKEA AAKEAAAKMI
PRGLSEAKPA TPEIQEIVDK VKPQLEEKTN ETYGKLEAVQ
YKTQVLAREG RQDWVLSTNY YIKVRAGDNK YMHLKVFNGP
WVPFPHQQLA DRVLTGYQVD KNKDDELTGF AEAAAKEAAA
KEAAAKEAAA KEAAAKEAAA KMIPRGLSEA KPATPEIQEI
VDKVKPQLEE KTNETYGKLE AVQYKTQVLA NFFQRRWPGS
TNYYIKVRAG DNKYMHLKVF NGPWKFRNTD RGADRVLTGY
QVDKNKDDEL TGFAAAGGRA EQKLISEEDL GAAGGGGSGG
GGSGGGGSGG GGSAPASSST KKTQLQLEHL LLDLQMILNG
INNYKNPKLT RMLTAKFAMP KKATELKHLQ CLEEELKPLE
EVLNGAQSKN FHLRPRDLIS NINVIVLELK GSETTFMCEY
ADETATIVEF LNRWITFAQS IISTLT
SEQ ID NO: 13
MPLLLLLPLL WAGALAIPRG LSEAKPATPE IQEIVDKVKP
QLEEKTNETY GKLEAVQYKT QVLAREGRQD WVLSTNYYIK
VRAGDNKYMH LKVFNGPWVP FPHQQLADRV LTGYQVDKNK
DDELTGFAEA AAKEAAAKEA AAKEAAAKEA AAKEAAAKMI
PRGLSEAKPA TPEIQEIVDK VKPQLEEKTN ETYGKLEAVQ
YKTQVLAREG RQDWVLSTNY YIKVRAGDNK YMHLKVFNGP
WVPFPHQQLA DRVLTGYQVD KNKDDELTGF AEAAAKEAAA
KEAAAKEAAA KEAAAKEAAA KMIPRGLSEA KPATPEIQEI
VDKVKPQLEE KTNETYGKLE AVQYKTQVLA NFFQRRWPGS
TNYYIKVRAG DNKYMHLKVF NGPWKFRNTD RGADRVLTGY
QVDKNKDDEL TGFAAAGGRA EQKLISEEDL GAAGGGGSGG
GGSGGGGSGG GGSAPTSSST KKTQLQLEHL LLDLQMILNG
INNYKNPKLT DMLTFEFYMP KKATELKHLQ CLERELKPLE
EVLNLAQSKN FHLRPRDLIS NINVIVLELK GSETTFMCEY
ADETATIVEF LNRWITFAQS IISTLT
SEQ ID NO: 14
MPLLLLLPLL WAGALAIPRG LSEAKPATPE IQEIVDKVKP
QLEEKTNETY GKLEAVQYKT QVLAREGRQD WVLSTNYYIK
VRAGDNKYMH LKVFNGPWVP FPHQQLADRV LTGYQVDKNK
DDELTGFAEA AAKEAAAKEA AAKEAAAKEA AAKEAAAKMI
PRGLSEAKPA TPEIQEIVDK VKPQLEEKTN ETYGKLEAVQ
YKTQVLAREG RQDWVLSTNY YIKVRAGDNK YMHLKVFNGP
WVPFPHQQLA DRVLTGYQVD KNKDDELTGF AEAAAKEAAA
KEAAAKEAAA KEAAAKEAAA KMIPRGLSEA KPATPEIQEI
VDKVKPQLEE KTNETYGKLE AVQYKTQVLA NFFQRRWPGS
TNYYIKVRAG DNKYMHLKVF NGPWKFRNTD RGADRVLTGY
QVDKNKDDEL TGFAAAGGRA EQKLISEEDL GAAGGGGSGG
GGSGGGGSGG GGSAPTSSST KKTQLQLEHL LLDLQMILNG
INNYKNPKLT AMLTAKFAMP KKATELKHLQ CLEEALKPLE
EVLNLAQSKN FHLRPRDLIS NINVIVLELK GSETTFMCEY
ADETATIVEF LNRWITFAQS IISTLT

In some embodiments of the additional polypeptide sequence can be a receptor ligand, such as is a ligand for a co-stimulatory receptor and agonizes the co-stimulatory receptor upon binding. For instance, the polypeptide ligand sequence can be selected from B7.1, 4-11B1L, OX40L, GITRL or LIGHT.

In some embodiments, where the additional polypeptide sequences require dimerization or higher order multimerization to work, the therapeutic protein may also include one or more multimerization domains that induces multimerization of, for example, the receptor ligand containing fusion protein or the cytokine containing fusion protein, i.e., complexes including 2, 3, 4, 5, 6, 7, 8, 9 or even 10 fusion proteins in a multimeric complex.

In still another aspect of the disclosure, the additional polypeptide sequence can be one that engages T-cells through binding, such as a CD3 binding polypeptide sequence that directs the fusion protein to bind to CD3 on the surface of T-cells.

Also provided herein, in some aspects, is a pharmaceutical composition suitable for therapeutic use in a human subject, comprising a chimeric protein of any of any one of the preceding claims, and a pharmaceutically acceptable excipient.

Further provided herein, in some aspects, is a polynucleotide comprising a sequence encoding a polypeptide (e.g., protein) of any of any one of the preceding claims. In some embodiments, the sequence encoding the polypeptide is operably linked to a transcriptional regulatory sequence. In some embodiments, the transcriptional regulatory sequence is selected from the group consisting of promoters and enhancers. In some embodiments, the polynucleotide further comprises an origin of replication, a minichromosome maintenance element (MME), and/or a nuclear localization element. In some embodiments, the polynucleotide further comprises a polyadenylation signal sequence operably linked and transcribed with the sequence encoding the polypeptide. In some embodiments, the sequence encoding the polypeptide comprises at least one intronic sequence. In some embodiments, the polynucleotide further comprises at least one ribosome binding site transcribed with the sequence encoding the polypeptide.

In some embodiments, the polynucleotide is a deoxyribonucleic acid (DNA). In some embodiments, the polynucleotide is a ribonucleic acid (RNA).

Also provided herein, in some aspects, is a viral vector comprising the polynucleotide of the present disclosure, a plasmid or minicircle comprising the polynucleotide of the present disclosure, a cell comprising the polypeptide of the present disclosure, the polynucleotide of the present disclosure, a viral vector of the present disclosure, and a plasmid or minicircle of the present disclosure.

Further aspects provide a method of producing a chimeric protein of the present disclosure, the method comprising expressing in a host cell a nucleic acid encoding the polypeptide, and optionally isolating the polypeptide from the host cell.

Also provided herein is a pharmaceutical composition suitable for therapeutic use in a human subject, comprising the chimeric protein of the present disclosure, and a pharmaceutically acceptable excipient.

In some embodiments, the HSA binding polypeptide comprises a loop 2 sequences selected from any one of SEQ ID NOs: 86-138 and/or a loop 4 sequence selected from any one of SEQ ID NOs: 139-191.

In some embodiments, the PD-L1 binding polypeptide comprises a loop 2 sequences selected from any one of SEQ ID NOs: 16-50 and/or a loop 4 sequence selected from any one of SEQ ID NOs: 51-85.

In some embodiments, the fusion protein comprises the amino acid sequence of SEQ ID NO: 277, or an amino acid sequence having at least 80%, at least 85%, at least 90%, or at least 95% identity to the amino acid sequence of SEQ ID NO: 277.

In some embodiments, the fusion protein comprises the amino acid sequence of SEQ ID NO: 278, or an amino acid sequence having at least 80%, at least 85%, at least 90%, or at least 95% identity to the amino acid sequence of SEQ ID NO: 278.

In some embodiments, the fusion protein comprises the amino acid sequence of SEQ ID NO: 279, or an amino acid sequence having at least 80%, at least 85%, at least 90%, or at least 95% identity to the amino acid sequence of SEQ ID NO: 279.

In some embodiments, the fusion protein comprises the amino acid sequence of SEQ ID NO: 280, or an amino acid sequence having at least 80%, at least 85%, at least 90%, or at least 95% identity to the amino acid sequence of SEQ ID NO: 280.

In some embodiments, the fusion protein comprises the amino acid sequence of SEQ ID NO: 281, or an amino acid sequence having at least 80%, at least 85%, at least 90%, or at least 95% identity to the amino acid sequence of SEQ ID NO: 281.

In some embodiments, the fusion protein comprises the amino acid sequence of SEQ ID NO: 282, or an amino acid sequence having at least 80%, at least 85%, at least 90%, or at least 95% identity to the amino acid sequence of SEQ ID NO: 282.

In some embodiments, the fusion protein further comprises a second a PD-L1 binding polypeptide, wherein the second PD-L1 binding polypeptide binds to PD-L1 with a K_{d} of 1×10⁻⁶M.

Some aspects of the present disclosure provide an in-line fusion protein comprising a human serum albumin (HSA) binding recombinantly engineered variant of stefin, first a PD-L1 binding polypeptide, and a second a PD-L1 binding polypeptide, wherein the HSA binding polypeptide binds to HSA with a K_{d} of 1×10⁻⁶M or less at pH 6.0 and optionally a K_{d} for binding HSA at pH 7.4 that is at least half a log greater than the K_{d} for binding at pH 6.0, and wherein the first and second PD-L1 binding polypeptides bind to PD-L1 with a K_{d} of 1×10⁻⁶M.

In some embodiments, the fusion protein further comprises a linker.

In some embodiments, the linker is a rigid linker or a flexible linker.

In some embodiments, the rigid linker comprises the sequence of SEQ ID NO: 294, or the flexible linker comprises the sequence of SEQ ID NO: 293.

In some embodiments, the fusion protein comprises the amino acid sequence of SEQ ID NO: 283, or an amino acid sequence having at least 80%, at least 85%, at least 90%, or at least 95% identity to the amino acid sequence of SEQ ID NO: 283.

In some embodiments, the fusion protein comprises the amino acid sequence of SEQ ID NO: 284, or an amino acid sequence having at least 80%, at least 85%, at least 90%, or at least 95% identity to the amino acid sequence of SEQ ID NO: 284.

In some embodiments, the fusion protein comprises the amino acid sequence of SEQ ID NO: 285, or an amino acid sequence having at least 80%, at least 85%, at least 90%, or at least 95% identity to the amino acid sequence of SEQ ID NO: 285.

In some embodiments, the fusion protein comprises the amino acid sequence of SEQ ID NO: 286, or an amino acid sequence having at least 80%, at least 85%, at least 90%, or at least 95% identity to the amino acid sequence of SEQ ID NO: 286.

In some embodiments, the fusion protein comprises the amino acid sequence of SEQ ID NO: 287, or an amino acid sequence having at least 80%, at least 85%, at least 90%, or at least 95% identity to the amino acid sequence of SEQ ID NO: 287.

In some embodiments, the fusion protein comprises the amino acid sequence of SEQ ID NO: 290, or an amino acid sequence having at least 80%, at least 85%, at least 90%, or at least 95% identity to the amino acid sequence of SEQ ID NO: 290.

In some embodiments, the fusion protein comprises the amino acid sequence of SEQ ID NO: 291, or an amino acid sequence having at least 80%, at least 85%, at least 90%, or at least 95% identity to the amino acid sequence of SEQ ID NO: 291.

In some embodiments, the linker is a flexible linker, optionally wherein the flexible linker comprises the sequence of SEQ ID NO: 293.

### BRIEF DESCRIPTION OF THE DRAWINGS

FIG. 1A Schematic representation of a PD-L1/serum albumin binding in-line fusion (ILF) AFFIMER® protein.

FIG. 1B SEC-HPLC chromatograms of PD-L1/serum albumin binding ILF AFFIMERS® following purification.

FIG. 2 Schematic representation of PD-L1/serum albumin binding trimer ILF AFFIMER® proteins.

FIG. 3 Production and SDS-PAGE analysis of purified PD-L1/serum albumin binding trimer ILF AFFIMER® proteins.

FIG. 4 BIACORE™ kinetic analysis showing ILF AFFIMER® trimers retain binding to both PD-L1 target antigen and serum albumin.

FIG. 5 Graph showing half-life extended AFFIMER® ILF trimers binding to human PD-L1 by ELISA and exhibiting similar binding to the parental molecule AVA04-251.

FIG. 6 Graph showing the potency of half-life extended ILF AFFIMER® polypeptides is similar to the parental molecule in the PD-1/PD-L1 blockade Bioassay (PROMEGA®).

FIG. 7 Graph showing the half-life extended ILF AFFIMER® polypeptides binding to human serum albumin binding is equivalent by ELISA at pH 7.4.

FIG. 8 Mixed lymphocyte reaction (MLR) showing ILF half-life extended AFFIMER® trimer (AVA04-251 XT14) is functional and retains potency when formatted compared to the parental molecule.

FIG. 9 Pharmacokinetic profile of ILF half-life extended trimers in mouse.

FIGS. 10A-10C In vivo efficacy of an ILF AVA04-251 XT14 in an A375 xenograft model. Individual traces over time are shown in FIG. 10A. FIG. 10B shows the results in FIG. 10A consolidated by group. FIG. 21C shows the tumor volume in each group.

FIG. 11 Expression and purification of AVA04-251 XT14-cys from E. coli.

FIG. 12A Anti-mouse PD-L1 AFFIMER® half-life extended trimer production and characterization.

FIG. 12B AVA04-182 XT20 K_{D} determination against mouse PD-L1 Fc using BIACORE™ FIGS. 13A and 13B ELISA showing AVA04-182 XT20 binding to MSA at pH 7.4 (FIG. 13A) and 6.0 (FIG. 13B).

FIG. 13C An mPD-L1 competition ELISA of both AVA04-182 and AVA04-182 XT20 FIG. 14 Pharmacokinetic profile of the AVA04-182 XT20 trimer, AVA04-182 Fc formatted AFFIMER® polypeptides in mice.

FIGS. 15A-15C Schematic (FIG. 15A) and characterization of AVA04-251 BH cys ILF dimer protein. FIG. 15B shows a purity analysis and FIG. 15C shows the SDS-PAGE analysis.

FIGS. 16A and 16B Evaluation of binding capacity of fluorescently labelled AFFIMER® polypeptides, AVA04-251 BH cys800 (FIG. 16A) and AVA04-251 XT14 cys800 (FIG. 16B), compared to parental molecules using a binding ELISA to huPD-L1.

FIG. 17 Representative images of biodistribution of fluorescently labelled AFFIMER® anti-huPD-L1 polypeptides in two A375 melanoma xenograft models four hours post treatment.

FIG. 18 Quality control analysis (purity) of AVA04-251 XT ILF with HSA-18 half-life extending AFFIMER® polypeptide (two different formats: XT60 and XT61).

FIGS. 19A and 19B Biacore kinetic analysis for the XT60 and XT61 ILF binding to HSA at pH7.4 (FIG. 19A) and pH6.0 (FIG. 19B).

FIG. 20 Binding ELISA for XT60 and XT61 ILF binding to HSA and MSA at pH7.4.

FIG. 21 XT60 and XT61 ILF Biacore kinetic analysis for binding to MSA at pH6.0.

FIG. 22 Biacore kinetic analysis for XT60 and XT61 ILF polypeptides binding to human PD-L1 Fc.

### DETAILED DESCRIPTION

The present disclosure is based on the generation of a chimeric protein that includes an AFFIMER® polypeptide that binds to PD-L1 and an AFFIMER® polypeptide that binds to human serum albumin (HSA). The HSA binding AFFIMER® polypeptide extends, in a controlled manner, the serum half-life of the PD-L1 binding AFFIMER® polypeptide to which it is conjugated.

Based on naturally occurring proteins (cystatins) that have been engineered to stably display two loops that create a binding surface, the AFFIMER® polypeptides of the present disclosure provide a number of advantages over antibodies, antibody fragments, and other non-antibody molecule-binding proteins. One is the small size of the AFFIMER® polypeptide itself. In its monomeric form it is about 14 kDa, or 1/10th the size of an antibody. This small size gives greater potential for increased tissue penetration, particularly in poorly vascularized and/or fibrotic target tissues (like tumors). AFFIMER® polypeptides have a simple protein structure (versus multi-domain antibodies), and as the AFFIMER® polypeptides do not require disulfide bonds or other post-translational modifications for function, these polypeptides can be manufactured in prokaryotic and eukaryotic systems.

Using libraries of AFFIMER® polypeptides (such as the phage display techniques described in the appended examples) as well as site directed mutagenesis, AFFIMER® polypeptides can be generated with tunable binding kinetics with ideal ranges for therapeutic uses. For instance, an AFFIMER® polypeptide can have high affinity for HSA or PD-L1, such as single digit nanomolar or lower K_{d} for monomeric AFFIMER® polypeptides, and picomolar K_{d} and avidity in multi-valent formats. An AFFIMER® polypeptide can be generated with tight binding kinetics for HSA or PD-L1, such as slow K_{off} rates in the 10-4 to 10⁻⁵ (s-1) range, which benefits target tissue localization.

The chimeric proteins of the present disclosure include AFFIMER® polypeptides with exquisite selectivity.

The lack of need for disulfide bonds and post-translational modifications also permit many embodiments of fusion proteins including the AFFIMER® polypeptides to be delivered therapeutically by expression of gene delivery constructs that are introduced into the tissues of a patient, including formats where the protein is delivered systemically (such as expression from muscle tissue) or delivered locally (such as through intratumoral gene delivery).

An AFFIMER® polypeptide (also referred to simply as an AFFIMER®) is a small, highly stable polypeptide (e.g., protein) that is a recombinantly engineered variant of stefin polypeptides. Thus, the term “AFFIMER® polypeptide” may be used interchangeably herein with the term “recombinantly engineered variant of stefin polypeptide”. A stefin polypeptide is a subgroup of proteins in the cystatin superfamily—a family that encompasses proteins containing multiple cystatin-like sequences. The stefin subgroup of the cystatin family is relatively small (˜100 amino acids) single domain proteins. They receive no known post-translational modification, and lack disulfide bonds, suggesting that they will be able to fold identically in a wide range of extracellular and intracellular environments. Stefin A is a monomeric, single chain, single domain protein of 98 amino acids. The structure of stefin A has been solved, facilitating the rational mutation of stefin A into the AFFIMER® polypeptide. The only known biological activity of cystatins is the inhibition of cathepsin activity, has enabled exhaustively testing for residual biological activity of the engineered proteins.

AFFIMER® polypeptides display two peptide loops and an N-terminal sequence that can all be randomized to bind to desired target proteins with high affinity and specificity, in a similar manner to monoclonal antibodies. Stabilization of the two peptides by the stefin A protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the stefin A polypeptide sequence can be carried out, with such variations improving the properties of these affinity reagents, such as increase stability, make them robust across a range of temperatures and pH, for example. In some embodiments, an AFFIMER® polypeptide includes a sequence derived from stefin A, sharing substantial identify with a stefin A wild type sequence, such as human stefin A. In some embodiments, an AFFIMER® polypeptide has an amino acid sequence that shares at least 25%, 35%, 45%, 55% or 60% identity to the sequences corresponding to human stefin A. For example, an AFFIMER® polypeptide may have an amino acid sequence that shares at least 70%, at least 80%, at least 85%, at least 90%, at least 92%, at least 94%, at least 95% identity, e.g., where the sequence variations do not adversely affect the ability of the scaffold to bind to the desired target, and e.g., which do not restore or generate biological functions such as those that are possessed by wild type stefin A, but which are abolished in mutational changes described herein.

### Bispecific AFFIMER® Proteins

One aspect of the disclosure provides chimeric proteins comprising an AFFIMER® polypeptide that binds programmed death-ligand 1 (PD-L1) and an AFFIMER® polypeptide that binds human serum albumin (HSA).

PD-L1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1 (Freeman et al., J. Exp. Med. 192(7): 1027-34 (2000); Latchm an et al., Nat Immunol 2:261-8 (2001)).

PD-1 primarily functions in peripheral tissues where activated T-cells may encounter the immunosuppressive PD-L1 (also called B7-H1 or CD274) and PD-L2 (B7-DC) ligands expressed by tumor and/or stromal cells (Flies et al., Yale J Biol Med 84:409-21 (2011); Topalian et al., Curr Opin Immuno 24:1-6 (2012)).

Inhibition of the PD-1/PD-L1 interaction mediates potent antitumor activity in preclinical models (U.S. Pat. Nos. 8,008,449 and 7,943,743). It appears that upregulation of PD-L1 may allow cancers to evade the host immune system. An analysis of 196 tumor specimens from patients with renal cell carcinoma found that high tumor expression of PD-L1 was associated with increased tumor aggressiveness and a 4.5-fold increased risk of death (Thompson et al., Proc Natl Acad Sci USA 101 (49): 17174-9 (2004)). Ovarian cancer patients with higher expression of PD-L1 had a significantly poorer prognosis than those with lower expression. PD-L1 expression correlated inversely with intraepithelial CD8+T-lymphocyte count, suggesting that PD-L1 on tumor cells may suppress antitumor CD8+ T cells (Hamanishi et al., Proc Natl Acad Sci USA 104 (9): 3360-3365 (2007)).

PD-L1 has also been implicated in infectious disease, in particular chronic infectious disease. Cytotoxic CD8 T lymphocytes (CTLs) play a pivotal role in the control of infection. Activated CTLs, however, often lose effector function during chronic infection. PD-1 receptor and its ligand PD-L1 of the B7/CD28 family function as a T cell co-inhibitory pathway and are emerging as major regulators converting effector CTLs into exhausted CTLs during chronic infection with human immunodeficiency virus, hepatitis B virus, hepatitis C virus, herpes virus, and other bacterial, protozoan, and viral pathogens capable of establishing chronic infections. Such bacterial and protozoal pathogens can include E. coli, Staphylococcus sp., Streptococcus sp., Mycobacterium tuberculosis, Giardia, Malaria, Leishmania, and Pseudomonas aeruginosa. Importantly, blockade of the PD-1/PD-L1 pathway is able to restore functional capabilities to exhausted CTLs. PD1/PD-L1 is thus a target for developing effective prophylactic and therapeutic vaccination against chronic bacterial and viral infections (see, e.g., Hofmeyer et al., Journal of Biomedicine and Biotechnology, vol. 2011, Article ID 451694, 9 pages, doi:10.1155/2011/451694).

Recent studies have also shown that systemic immune suppression may curtail the ability to mount the protective, cell-mediated immune responses that are needed for brain repair in neurodegenerative diseases. By using mouse models of Alzheimer's disease, immune checkpoint blockade directed against the programmed death-1 (PD-1) pathway was shown to evoke an interferon γ-dependent systemic immune response, which was followed by the recruitment of monocyte-derived macrophages to the brain. When induced in mice with established pathology, this immunological response led to clearance of cerebral amyloid-β (Aβ) plaques and improved cognitive performance. These findings suggest that immune checkpoints may be targeted therapeutically in neurodegenerative disease such as Alzheimer's disease using antibodies to PD-L1 (see, e.g., Baruch et al., Nature Medicine, January 2016, doi:10.1038/nm.4022).

Human serum albumin (HSA) is a protein encoded by the ALB gene. HSA is a 585 amino acid polypeptide (approx. 67 kDa) having a serum half-life of about 20 days, and is primarily responsible for the maintenance of colloidal osmotic blood pressure, blood pH, and transport and distribution of numerous endogenous and exogenous ligands. HSA has three structurally homologous domains (domains I, II and III), is almost entirely in the alpha-helical conformation, and is highly stabilized by 17 disulfide bridges. A representative HSA sequence is provided by UniProtKB Primary accession number P02768 and may include other human isoforms thereof.

AFFIMER® polypeptides comprise an AFFIMER® polypeptide in which at least one of the solvent accessible loops is from the wild-type stefin A protein having amino acid sequences to enable an AFFIMER® polypeptide to bind PD-L1 or HSA, selectively, and in some embodiments, with a K_{d} of 10⁻⁶M or less.

In some embodiments, an AFFIMER® polypeptide bind to PD-L1 or HSA with a K_{d} of 1×10⁻⁹ M to 1×10⁻⁶ M at pH 7.4 to 7.6. In some embodiments, the polypeptides bind to HSA with a K_{d} of 1×10⁻⁶ M or less at pH 7.4 to 7.6. In some embodiments, an AFFIMER® polypeptide bind to PD-L1 or HSA with a K_{d} of 1×10⁻⁷ M or less at pH 7.4 to 7.6. In some embodiments, an AFFIMER® polypeptide bind to PD-L1 or HSA with a K_{d} of 1×10⁻⁸ M or less at pH 7.4 to 7.6. In some embodiments, an AFFIMER® polypeptide bind to PD-L1 or HSA with a K_{d} of 1×10⁻⁹ M or less at pH 7.4 to 7.6. In some embodiments, an AFFIMER® polypeptide bind to PD-L1 or HSA with a K_{d} of 1×10⁻⁹ M to 1×10⁻⁶ M at pH 7.4. In some embodiments, an AFFIMER® polypeptide bind to PD-L1 or HSA with a K_{d} of 1×10⁻⁶ M or less at pH 7.4. In some embodiments, an AFFIMER® polypeptide bind to PD-L1 or HSA with a K_{d} of 1×10⁻⁷ M or less at pH 7.4. In some embodiments, an AFFIMER® polypeptide bind to PD-L1 or HSA with a K_{d} of 1×10⁻⁸ M or less at pH 7.4. In some embodiments, an AFFIMER® polypeptide bind to PD-L1 or HSA with a K_{d} of 1×10⁻⁹ M or less at pH 7.4.

In some embodiments, an AFFIMER® polypeptide at pH 5.8 to 6.2 binds to HSA with a K_{d} of half a log to 2.5 logs less than the K_{d} for binding to HSA at pH 7.4 to 7.6, respectively. In some embodiments, an AFFIMER® polypeptide at pH 5.8 to 6.2 binds to HSA with a K_{d} of half a log less than the K_{d} for binding to HSA at pH 7.4 to 7.6, respectively. In some embodiments, an AFFIMER® polypeptide at pH 5.8 to 6.2 binds to HSA with a K_{d} of at least one log less than the K_{d} for binding to HSA at pH 7.4 to 7.6, respectively. In some embodiments, an AFFIMER® polypeptide at pH 5.8 to 6.2 binds to HSA with a K_{d} of at least 1.5 log less than the K_{d} for binding to HSA at pH 7.4 to 7.6, respectively. In some embodiments, an AFFIMER® polypeptide at pH 5.8 to 6.2 binds to HSA with a K_{d} of at least 2 log less than the K_{d} for binding to HSA at pH 7.4 to 7.6, respectively. In some embodiments, an AFFIMER® polypeptide at pH 5.8 to 6.2 binds to HSA with a K_{d} of at least 2.5 log less than the K_{d} for binding to HSA at pH 7.4 to 7.6, respectively.

In some embodiments, an AFFIMER® polypeptide at pH 6 binds to HSA with a K_{d} of half a log to 2.5 logs less than the K_{d} for binding to HSA at pH 7.4. In some embodiments, an AFFIMER® polypeptide at pH 6 binds to HSA with a K_{d} of at least half a log less than the K_{d} for binding to HSA at pH 7.4. In some embodiments, an AFFIMER® polypeptide at pH 6 binds to HSA with a K_{d} of at least one log less than the K_{d} for binding to HSA at pH 7.4. In some embodiments, an AFFIMER® polypeptide at pH 6 binds to HSA with a K_{d} of at least 1.5 log less than the K_{d} for binding to HSA at pH 7.4. In some embodiments, an AFFIMER® polypeptide at pH 6 binds to HSA with a K_{d} of at least two logs less than the K_{d} for binding to HSA at pH 7.4. In some embodiments, an AFFIMER® polypeptide at pH 6 binds to HSA with a K_{d} of at least 2.5 logs less than the K_{d} for binding to HSA at pH 7.4.

In some embodiments, the proteins comprising the AFFIMER® polypeptides have a serum half-life in human patients of greater than 10 hours. In some embodiments, the polypeptides have a serum half-life in human patients of greater than 24 hours. In some embodiments, the proteins comprising the AFFIMER® polypeptides have a serum half-life in human patients of greater than 48 hours. In some embodiments, the proteins comprising the AFFIMER® polypeptides have a serum half-life in human patients of greater than 72 hours. In some embodiments, the proteins comprising the AFFIMER® polypeptides have a serum half-life in human patients of greater than 96 hours. In some embodiments, the proteins comprising the AFFIMER® polypeptides have a serum half-life in human patients of greater than 120 hours. In some embodiments, the proteins comprising the AFFIMER® polypeptides have a serum half-life in human patients of greater than 144 hours. In some embodiments, the proteins comprising the AFFIMER® polypeptides have a serum half-life in human patients of greater than 168 hours. In some embodiments, the proteins comprising the AFFIMER® polypeptides have a serum half-life in human patients of greater than 192 hours. In some embodiments, the proteins comprising the AFFIMER® polypeptides have a serum half-life in human patients of greater than 216 hours. In some embodiments, the proteins comprising the AFFIMER® polypeptides have a serum half-life in human patients of greater than 240 hours. In some embodiments, the proteins comprising the AFFIMER® polypeptides have a serum half-life in human patients of greater than 264 hours. In some embodiments, the proteins comprising the AFFIMER® polypeptides have a serum half-life in human patients of greater than 288 hours. In some embodiments, the proteins comprising the AFFIMER® polypeptides have a serum half-life in human patients of greater than 312 hours. In some embodiments, the proteins comprising the AFFIMER® polypeptides have a serum half-life in human patients of greater than 336 hours. In some embodiments, the proteins comprising the AFFIMER® polypeptides have a serum half-life in human patients of greater than 360 hours. In some embodiments, the proteins comprising the AFFIMER® polypeptides have a serum half-life in human patients of 24 to 360 hours, 48 to 360 hours, 72 to 360 hours, 96 to 360 hours, or 120 to 360 hours.

In some embodiments, the polypeptides have a serum half-life in human patients of greater than 50%, greater than 60%, greater than 70%, or greater than 80% of the serum half-life of HSA. In some embodiments, the polypeptides have a serum half-life in human patients of 50% to 80%, 50% to 90%, or 50% to 100% of the serum half-life of HSA.

In some embodiments, AFFIMER® polypeptides (HSA or PD-L1 binding AFFIMER® polypeptides) have amino acid sequences which are, each independently, represented in general formula (I):

FR1-(Xaa)ₙ-FR2-(Xaa)ₘ-FR3 (I)

wherein
FR1 is an amino acid sequence having at least 70% identity to MIPGGLSEAK PATPEIQEIV DKVKPQLEEK TNETYGKLEA VQYKTQVLA (SEQ ID NO: 1) or MIPRGLSEAK PATPEIQEIV DKVKPQLEEK TNETYGKLEA VQYKTQVLA (SEQ ID NO: 2);
FR2 is an amino acid sequence having at least 70% identity to GTNYYIKVRA GDNKYMHLKV FKSL (SEQ ID NO: 3) or STNYYIKVRA GDNKYMHLKV FNGP (SEQ ID NO: 4);
FR3 is an amino acid sequence having at least 70% identity to ADRVLTGYQV DKNKDDELTG F (SEQ ID NO: 5) or EDLVLTGYQV DKNKDDELTG F (SEQ ID NO: 6);
Xaa, individually for each occurrence, is an amino acid,
n is an integer from 3 to 20, and
m is an integer from 3 to 20.

In some embodiments, FR1 is a polypeptide sequence having at least 80%, 85%, 90%, 95% or even 98% homology with SEQ ID NO: 1 and/or 2. In some embodiments, FR1 is a polypeptide sequence having at least 80%, 85%, 90%, 95% or even 98% identity with SEQ ID NO: 1 and/or 2; In some embodiments, FR2 is a polypeptide sequence having at least 80%, 85%, 90%, 95% or even 98% homology with SEQ ID NO: 3 and/or 4. In some embodiments, FR2 is a polypeptide sequence having at least 80%, 85%, 90%, 95% or even 98% identity with SEQ ID NO: 3 and/or 4; In some embodiments, FR3 is a polypeptide sequence having at least 80%, 85%, 90%, 95% or even 98% homology with SEQ ID NO: 5 and/or 6. In some embodiments, FR3 is a polypeptide sequence having at least 80%, 85%, 90%, 95% or even 98% identity with SEQ ID NO: 5 and/or 6.

In some embodiments, a PD-L1 binding AFFIMER® polypeptide comprises a loop 2 amino acid sequence selected from any one of SEQ ID NOs: 16-50 (Table 1). In some embodiments, a PD-L1 binding AFFIMER® polypeptide comprises a loop 4 amino acid sequence selected from any one of SEQ ID NOs: 51-85 (Table 1).

In some embodiments, (Xaa)ₙ comprises an amino acid sequence having at least 80% or at least 90% identity to the amino acid sequence of any one of SEQ ID NOs: 16-50. In some embodiments, (Xaa)ₙ comprises an amino acid sequence having 80% to 90% identity to the amino acid sequence of any one of SEQ ID NOs: 16-50. In some embodiments, (Xaa)ₙ comprises the amino acid sequence of any one of SEQ ID NOs: 16-50.

In some embodiments, (Xaa)ₘ comprises an amino acid sequence having at least 80% or at least 90% identity to the amino acid sequence of any one of SEQ ID NOs: 51-85. In some embodiments, (Xaa)ₘ comprises an amino acid sequence having 80% to 90% identity to the amino acid sequence of any one of SEQ ID NOs: 51-85. In some embodiments, (Xaa)ₘ comprises the amino acid sequence of any one of SEQ ID NOs: 51-85.

In some embodiments, an HSA binding AFFIMER® polypeptide comprises a loop 2 amino acid sequence selected from any one of SEQ ID NOs: 86-138 (Table 2). In some embodiments, an HSA binding AFFIMER® polypeptide comprises a loop 4 amino acid sequence selected from any one of SEQ ID NOs: 86-138 (Table 2).

In some embodiments, (Xaa)₁₁ comprises an amino acid sequence having at least 80% or at least 90% identity to the amino acid sequence of any one of SEQ ID NOs: 86-138. In some embodiments, (Xaa)₁₁ comprises an amino acid sequence having 80% to 90% identity to the amino acid sequence of any one of SEQ ID NOs: 86-138. In some embodiments, (Xaa) comprises the amino acid sequence of any one of SEQ ID NOs: 86-138.

In some embodiments, (Xaa)ₘ comprises an amino acid sequence having at least 80% or at least 90% identity to the amino acid sequence of any one of SEQ ID NOs: 139-191. In some embodiments, (Xaa)ₘ comprises an amino acid sequence having 80% to 90% identity to the amino acid sequence of any one of SEQ ID NOs: 139-191. In some embodiments, (Xaa)ₘ comprises the amino acid sequence of any one of SEQ ID NOs: 139-191.

TABLE 1
Examples of PD-L1 Binding AFFIMER ® Loop Sequences

| | | SEQ | | SEQ |
|---|---|---|---|---|
| Name | Loop 2 | ID NO: | Loop 4 | ID NO: |
| PD-L1-01 | KAWGPKQWW | 16 | GRTIQ | 51 |
| PD-L1-02 | KPYGPRDWD | 17 | EPQLDTSPI | 52 |
| PD-L1-03 | KEYGPEEWW | 18 | GDYEQVLIH | 53 |
| PD-L1-04 | HAYGPRDWD | 19 | PADHVLEEA | 54 |
| PD-L1-05 | KDHGPIAWW | 20 | EDTNTDGAL | 55 |
| PD-L1-06 | NKHFHQRFW | 21 | GQSWDQRRQ | 56 |
| PD-L1-07 | NKHFPIHFW | 22 | SKSPIDLPF | 57 |
| PD-L1-08 | HEFGPAEWD | 23 | DPQDVYLNQ | 58 |
| PD-L1-09 | NAHFPQSFW | 24 | GSLHSFGST | 59 |
| PD-L1-00 | KEHGPDSWW | 25 | QEKNQWVEE | 60 |
| PD-L1-11 | NQHFPHSFW | 26 | QKNYEEDPH | 61 |
| PD-L1-12 | NAHFGPRFW | 27 | WDGHKRFAD | 62 |
| PD-L1-13 | NTWFPESFW | 28 | DDNQERQEH | 63 |
| PD-L1-14 | NQHFPQSFW | 29 | AVTQEDQAV | 64 |
| PD-L1-15 | KQYGPDDWW | 30 | EVDWKYQDH | 65 |
| PD-L1-16 | KDWGPSNWW | 31 | VDDKTLSKD | 66 |
| PD-L1-17 | KQFGPKDWW | 32 | QGQGKDPSQ | 67 |
| PD-L1-18 | NHHFPKRFW | 33 | GHQSEVQHS | 68 |
| PD-L1-19 | YRHFPQWH | 34 | TGTSIWNQD | 69 |
| PD-L1-20 | NIHFPPNFW | 35 | GVHDSLQGYDA | 70 |
| PD-L1-21 | YTHFPQWT | 36 | QKGQKIDKF | 71 |
| PD-L1-22 | NDHFPHTFW | 37 | DDELHDTRH | 72 |
| PD-L1-23 | NQHFPSYFW | 38 | ATTGDEWDR | 73 |
| PD-L1-24 | NQYFPPHFW | 39 | SHPHSNHTS | 74 |
| PD-L1-25 | KKHFPASFW | 40 | WRTDYKYEE | 75 |
| PD-L1-26 | KKFFPKHFW | 41 | NDPHDSVPH | 76 |
| PD-L1-27 | KLHFPRSFW | 42 | GQQRENEQE | 77 |
| PD-L1-28 | YKHFPPNFW | 43 | GERQQDDAN | 78 |
| PD-L1-29 | EEHFPFQFW | 44 | AYREGSQWT | 79 |
| PD-L1-30 | KPHFPDNFW | 45 | EFYDHGIIQ | 80 |
| PD-L1-31 | YQYFPDQFN | 46 | ENEATRDQH | 81 |
| PD-L1-32 | VQWFPRSFW | 47 | GYDHEDNRG | 82 |
| PD-L1-33 | AAHFPEHFW | 48 | QPADMSAEF | 83 |
| PD-L1-34 | REGRQDWVL | 49 | WVPFPHQQL | 84 |
| PD-L1-35 | WVPFPHQQL | 50 | REGRQDWVL | 85 |

TABLE 2
Examples of HSA Binding AFFIMER ® Loop Sequences

| | | SEQ ID | | SEQ ID |
|---|---|---|---|---|
| Name | Loop 2 | NO: | Loop 4 | NO: |
| HSA-00 | WTQPKNEHH | 86 | RFKYFAHYQ | 139 |
| HSA-01 | HLKHTDAQP | 87 | FHDFWHRRW | 140 |
| HSA-02 | HDQDVLHAW | 88 | DWYHYWWEV | 141 |
| HSA-03 | KFHRQEWAD | 89 | STRSIHVTT | 142 |
| HSA-04 | PEDFWDPEH | 90 | KQHHHYLDK | 143 |
| HSA-05 | VVRTTGHVV | 91 | HSAQDREIP | 144 |
| HSA-06 | YWWFCTGQS | 92 | WVQSGYNSQ | 145 |
| HSA-07 | IHHRQARSL | 93 | AVFWGKWSD | 146 |
| HSA-08 | SHRRRAYIW | 94 | QSFDKPWTT | 147 |
| HSA-09 | WDSHHWRAP | 95 | HYPLKYSFE | 148 |
| HSA-10 | DKRVKYGQ | 96 | WHHPWHRNR | 149 |
| HSA-11 | SDWVYALQL | 97 | DPWWAWVVW | 150 |
| HSA-12 | FWWFWY | 98 | FDNQDLIQY | 151 |
| HSA-13 | VRDWPWNTF | 99 | EKKNWYKWD | 152 |
| HSA-14 | QKKRDEDYI | 100 | DRHKSRWGI | 153 |
| HSA-15 | GVHEEPRKL | 101 | LNPFTPSVT | 154 |
| HSA-16 | EWWQKHWPS | 102 | YKGALLNHD | 155 |
| HSA-17 | NFFQRRWPG | 103 | WKFRNTERG | 156 |
| HSA-18 | DWWQAKWPH | 104 | YKVHQSSGG | 157 |
| HSA-19 | GIWQSRWPG | 105 | FHPIAGRPW | 158 |
| HSA-20 | GYWAAKWPG | 106 | FPNTSYDLQ | 159 |
| HSA-21 | GFYADHWPG | 107 | FAHYNLKSG | 160 |
| HSA-22 | NWYQQRWPG | 108 | WHNYGESSG | 161 |
| HSA-23 | GFYARHWPG | 109 | KFYYADHQW | 162 |
| HSA-24 | DFWKAHWPG | 110 | YTHADPHSQ | 163 |
| HSA-25 | DFYSVRWPG | 111 | FGVPQLGAG | 164 |
| HSA-26 | YWAANHASK | 112 | YSGFPFAGF | 165 |
| HSA-27 | IKRLEHWEY | 113 | WFSWPYTPL | 166 |
| HSA-28 | EWDSPWSEN | 114 | YYHPSIQST | 167 |
| HSA-29 | KHKNLRWPF | 115 | FLGWKDTVV | 168 |
| HSA-30 | RHFPKQTNW | 116 | DWWKWWWAK | 169 |
| HSA-31 | VWGPEYQHQ | 117 | NAGWPLVPE | 170 |
| HSA-32 | TWKNNGQDV | 118 | YALDPFGGK | 171 |
| HSA-33 | ATWLNYYLP | 119 | GYKFWGVSD | 172 |
| HSA-34 | DQESLFLNN | 120 | QGKQYILLR | 173 |
| HSA-35 | GFYAQHWPD | 121 | YKRHSAHDY | 174 |
| HSA-36 | GHYARYWPG | 122 | WAQKSKVHQ | 175 |
| HSA-37 | GFWASKWPG | 123 | FTAVSKKDA | 176 |
| HSA-38 | GFWQRKWPN | 124 | WGDKENIWE | 177 |
| HSA-39 | VWPADNDLK | 125 | WSGHPWVQK | 178 |
| HSA-40 | HWAWTSPGY | 126 | YADYPLSPK | 179 |
| HSA-41 | NFFQRRWPG | 127 | WKFRNTDRG | 180 |
| HSA-42 | HHSHRLKGQ | 128 | QTVATHYHY | 181 |
| HSA-43 | YQNTIFLSI | 129 | WHAKHLLSH | 182 |
| HSA-44 | FQDQFTWSQ | 130 | SGIKKADSV | 183 |
| HSA-45 | GEPHWPWQA | 131 | KANLINVKS | 184 |
| HSA-46 | ADPRHPWVE | 132 | WKSHVEVRS | 185 |
| HSA-47 | FHKRFQSQG | 133 | WVTQKYIIQ | 186 |
| HSA-48 | EWWQNRWPN | 134 | WEHAKDWPT | 187 |
| HSA-49 | EWYQTRWPG | 135 | FHSKVLDKA | 188 |
| HSA-50 | EFWQRHWPG | 136 | YGAQKQAVW | 189 |
| HSA-51 | KFYERHWPG | 137 | FSASHFTSQ | 190 |
| Consensus | GWWQRRWPG | 138 | X₁X₂AX₃KX₄DX₅Q | 191 |

In some embodiments, an PD-L1 binding AFFIMER® polypeptide comprises an amino acid sequence selected from any one of SEQ ID NOs: 192-200 (Table 3).

In some embodiments, an PD-L1 binding AFFIMER® polypeptide comprises an amino acid sequence having at least 80% or at least 90% identity to the amino acid sequence of any one of SEQ ID NOs: 192-200. In some embodiments, an PD-L1 binding AFFIMER® polypeptide comprises an amino acid sequence having 80% to 90% identity to the amino acid sequence of any one of SEQ ID NOs: 192-200. In some embodiments, an PD-L1 binding AFFIMER® polypeptide comprises the amino acid sequence of any one of SEQ ID NOs: 192-200.

In some embodiments, an HSA binding AFFIMER® polypeptide comprises an amino acid sequence selected from any one of SEQ ID NOs: 201-235 (Table 4).

In some embodiments, an HSA binding AFFIMER® polypeptide comprises an amino acid sequence having at least 80% or at least 90% identity to the amino acid sequence of any one of SEQ ID NOs: 201-235. In some embodiments, an HSA binding AFFIMER® polypeptide comprises an amino acid sequence having 80% to 90% identity to the amino acid sequence of any one of SEQ ID NOs: 201-235. In some embodiments, an HSA binding AFFIMER® polypeptide comprises the amino acid sequence of any one of SEQ ID NOs: 201-235.

TABLE 3
Examples of PD-L1 Binding AFFIMER ® Polypeptide Sequences

| Protein Sequence | SEQ ID NO: |
|---|---|
| MIPRGLSEAK PATPEIQEIV DKVKPQLEEK TNETYGKLEA VQYKTQVLAK EHGPDSWWST | 192 |
| NYYIKVRAGD NKYMHLKVFN GPQEKNQWVE EADRVLTGYQ VDKNKDDELT GF | |
| MIPRGLSEAK PATPEIQEIV DKVKPQLEEK TNETYGKLEA VQYKTQVLAK EYGPEEWWST | 193 |
| NYYIKVRAGD NKYMHLKVFN GPGDYEQVLI HADRVLTGYQ VDKNKDDELT GF | |
| MIPRGLSEAK PATPEIQEIV DKVKPQLEEK TNETYGKLEA VQYKTQVLAK DHGPIAWWST | 194 |
| NYYIKVRAGD NKYMHLKVFN GPEDTNTDGA LADRVLTGYQ VDKNKDDELT GF | |
| MIPRGLSEAK PATPEIQEIV DKVKPQLEEK TNETYGKLEA VQYKTQVLAK DWGPSNWWST | 195 |
| NYYIKVRAGD NKYMHLKVFN GPVDDKTLSK DADRVLTGYQ VDKNKDDELT GF | |
| MIPRGLSEAK PATPEIQEIV DKVKPQLEEK TNETYGKLEA VQYKTQVLAN TWFPESFWST | 196 |
| NYYIKVRAGD NKYMHLKVFN GPDDNQERQE HADRVLTGYQ VDKNKDDELT GF | |
| MIPRGLSEAK PATPEIQEIV DKVKPQLEEK TNETYGKLEA VQYKTQVLAK PYGPRDWDST | 197 |
| NYYIKVRAGD NKYMHLKVFN GPEPQLDTSP IADRVLTGYQ VDKNKDDELT GF | |
| MIPRGLSEAK PATPEIQEIV DKVKPQLEEK TNETYGKLEA VQYKTQVLAH AYGPRDWDST | 198 |
| NYYIKVRAGD NKYMHLKVFN GPPADHVLEE AADRVLTGYQ VDKNKDDELT GF | |
| MIPRGLSEAK PATPEIQEIV DKVKPQLEEK TNETYGKLEA VQYKTQVLAA AHFPEHFWST | 199 |
| NYYIKVRAGD NKYMHLKVFN GPQPADMSAE FADRVLTGYQ VDKNKDDELT GF | |
| MIPRGLSEAK PATPEIQEIV DKVKPQLEEK TNETYGKLEA VQYKTQVLAR EGRQDWVLST | 200 |
| NYYIKVRAGD NKYMHLKVFN GPWVPFPHQQ LADRVLTGYQ VDKNKDDELT GF | |

TABLE 4
Examples of HSA Binding AFFIMER ® Polypeptide Sequences

| Name | Sequence | SEQ ID NO: |
|---|---|---|
| HSA-18 | MIPRGLSEAKPATPEIQEIVDKVKPQLEEKTNETYGKLEAVQYKTQVLADW | 201 |
| | WQAKWPHSTNYYIKVRAGDNKYMHLKVFNGPYKVHQSSGGADRVLTGYQVD | |
| | KNKDDELTGF | |
| HSA-19 | MIPRGLSEAKPATPEIQEIVDKVKPQLEEKTNETYGKLEAVQYKTQVLAGI | 202 |
| | WQSRWPGSTNYYIKVRAGDNKYMHLKVFNGPFHPIAGRPWADRVLTGYQVD | |
| | KNKDDELTGF | |
| HSA-20 | MIPRGLSEAKPATPEIQEIVDKVKPQLEEKTNETYGKLEAVQYKTQVLAGY | 203 |
| | WAAKWPGSTNYYIKVRAGDNKYMHLKVFNGPFPNTSYDLQADRVLTGYQVD | |
| | KNKDDELTGF | |
| HSA-21 | MIPRGLSEAKPATPEIQEIVDKVKPQLEEKTNETYGKLEAVQYKTQVLAGF | 204 |
| | YADHWPGSTNYYIKVRAGDNKYMHLKVFNGPFAHYNLKSGADRVLTGYQVD | |
| | KNKDDELTGF | |
| HSA-22 | MIPRGLSEAKPATPEIQEIVDKVKPQLEEKTNETYGKLEAVQYKTQVLANW | 205 |
| | YQQRWPGSTNYYIKVRAGDNKYMHLKVFNGPWHNYGESSGADRVLTGYQVD | |
| | KNKDDELTGF | |
| HSA-23 | MIPRGLSEAKPATPEIQEIVDKVKPQLEEKTNETYGKLEAVQYKTQVLAGF | 206 |
| | YARHWPGSTNYYIKVRAGDNKYMHLKVFNGPKFYYADHQWADRVLTGYQVD | |
| | KNKDDELTGF | |
| HSA-24 | MIPRGLSEAKPATPEIQEIVDKVKPQLEEKTNETYGKLEAVQYKTQVLADF | 207 |
| | WKAHWPGSTNYYIKVRAGDNKYMHLKVFNGPYTHADPHSQADRVLTGYQVD | |
| | KNKDDELTGF | |
| HSA-25 | MIPRGLSEAKPATPEIQEIVDKVKPQLEEKTNETYGKLEAVQYKTQVLADF | 208 |
| | YSVRWPGSTNYYIKVRAGDNKYMHLKVFNGPFGVPQLGAGADRVLTGYQVD | |
| | KNKDDELTGF | |
| HSA-26 | MIPRGLSEAKPATPEIQEIVDKVKPQLEEKTNETYGKLEAVQYKTQVLAYW | 209 |
| | AANHASKSTNYYIKVRAGDNKYMHLKVFNGPYSGFPFAGFADRVLTGYQVD | |
| | KNKDDELTGF | |
| HSA-27 | MIPRGLSEAKPATPEIQEIVDKVKPQLEEKTNETYGKLEAVQYKTQVLAIK | 210 |
| | RLEHWEYSTNYYIKVRAGDNKYMHLKVFNGPWFSWPYTPLADRVLTGYQVD | |
| | KNKDDELTGF | |
| HSA-28 | MIPRGLSEAKPATPEIQEIVDKVKPQLEEKTNETYGKLEAVQYKTQVLAEW | 211 |
| | DSPWSENSTNYYIKVRAGDNKYMHLKVFNGPYYHPSIQSTADRVLTGYQVD | |
| | KNKDDELTGF | |
| HSA-29 | MIPRGLSEAKPATPEIQEIVDKVKPQLEEKTNETYGKLEAVQYKTQVLAKH | 212 |
| | KNLRWPFSTNYYIKVRAGDNKYMHLKVFNGPFLGWKDTVVADRVLTGYQVD | |
| | KNKDDELTGF | |
| HSA-30 | MIPRGLSEAKPATPEIQEIVDKVKPQLEEKTNETYGKLEAVQYKTQVLARH | 213 |
| | FPKQTNWSTNYYIKVRAGDNKYMHLKVFNGPDWWKWWWAKADRVLTGYQVD | |
| | KNKDDELTGF | |
| HSA-31 | MIPRGLSEAKPATPEIQEIVDKVKPQLEEKTNETYGKLEAVQYKTQVLAVW | 214 |
| | GPEYQHQSTNYYIKVRAGDNKYMHLKVFNGPNAGWPLVPEADRVLTGYQVD | |
| | KNKDDELTGF | |
| HSA-32 | MIPRGLSEAKPATPEIQEIVDKVKPQLEEKTNETYGKLEAVQYKTQVLATW | 215 |
| | KNNGQDVSTNYYIKVRAGDNKYMHLKVFNGPYALDPFGGKADRVLTGYQVD | |
| | KNKDDELTGF | |
| HSA-33 | MIPRGLSEAKPATPEIQEIVDKVKPQLEEKTNETYGKLEAVQYKTQVLAAT | 216 |
| | WLNYYLPSTNYYIKVRAGDNKYMHLKVFNGPGYKFWGVSDADRVLTGYQVD | |
| | KNKDDELTGF | |
| HSA-34 | MIPRGLSEAKPATPEIQEIVDKVKPQLEEKTNETYGKLEAVQYKTQVLADQ | 217 |
| | ESLFLNNSTNYYIKVRAGDNKYMHLKVFNGPQGKQYILLRADRVLTGYQVD | |
| | KNKDDELTGF | |
| HSA-35 | MIPRGLSEAKPATPEIQEIVDKVKPQLEEKTNETYGKLEAVQYKTQVLAGF | 218 |
| | YAQHWPDSTNYYIKVRAGDNKYMHLKVFNGPYKRHSAHDYADRVLTGYQVD | |
| | KNKDDELTGF | |
| HSA-36 | MIPRGLSEAKPATPEIQEIVDKVKPQLEEKTNETYGKLEAVQYKTQVLAGH | 219 |
| | YARYWPGSTNYYIKVRAGDNKYMHLKVFNGPWAQKSKVHQADRVLTGYQVD | |
| | KNKDDELTGF | |
| HSA-37 | MIPRGLSEAKPATPEIQEIVDKVKPQLEEKTNETYGKLEAVQYKTQVLAGF | 220 |
| | WASKWPGSTNYYIKVRAGDNKYMHLKVFNGPFTAVSKKDAADRVLTGYQVD | |
| | KNKDDELTGF | |
| HSA-38 | MIPRGLSEAKPATPEIQEIVDKVKPQLEEKTNETYGKLEAVQYKTQVLAGF | 221 |
| | WQRKWPNSTNYYIKVRAGDNKYMHLKVFNGPWGDKENIWFADRVLTGYQVD | |
| | KNKDDELTGF | |
| HSA-39 | MIPRGLSEAKPATPEIQEIVDKVKPQLEEKTNETYGKLEAVQYKTQVLAVW | 222 |
| | PADNDLKSTNYYIKVRAGDNKYMHLKVFNGPWSGHPWVQKADRVLTGYQVD | |
| | KNKDDELTGF | |
| HSA-40 | MIPRGLSEAKPATPEIQEIVDKVKPQLEEKTNETYGKLEAVQYKTQVLAHW | 223 |
| | AWTSPGYSTNYYIKVRAGDNKYMHLKVFNGPYADYPLSPKADRVLTGYQVD | |
| | KNKDDELTGF | |
| HSA-41 | MIPRGLSEAKPATPEIQEIVDKVKPQLEEKTNETYGKLEAVQYKTQVLANF | 224 |
| | FQRRWPGSTNYYIKVRAGDNKYMHLKVFNGPWKFRNTDRGADRVLTGYQVD | |
| | KNKDDELTGF | |
| HSA-42 | MIPRGLSEAKPATPEIQEIVDKVKPQLEEKTNETYGKLEAVQYKTQVLAHH | 225 |
| | SHRLKGQSTNYYIKVRAGDNKYMHLKVFNGPQTVATHYHYADRVLTGYQVD | |
| | KNKDDELTGF | |
| HSA-43 | MIPRGLSEAKPATPEIQEIVDKVKPQLEEKTNETYGKLEAVQYKTQVLAYQ | 226 |
| | NTIFLSISTNYYIKVRAGDNKYMHLKVFNGPWHAKHLLSHADRVLTGYQVD | |
| | KNKDDELTGF | |
| HSA-44 | MIPRGLSEAKPATPEIQEIVDKVKPQLEEKTNETYGKLEAVQYKTQVLAFQ | 227 |
| | DQFTWSQSTNYYIKVRAGDNKYMHLKVFNGPSGIKKADSVADRVLTGYQVD | |
| | KNKDDELTGF | |
| HSA-45 | MIPRGLSEAKPATPEIQEIVDKVKPQLEEKTNETYGKLEAVQYKTQVLAGE | 228 |
| | PHWPWQASTNYYIKVRAGDNKYMHLKVFNGPKANLINVKSADRVLTGYQVD | |
| | KNKDDELTGF | |
| HSA-46 | MIPRGLSEAKPATPEIQEIVDKVKPQLEEKTNETYGKLEAVQYKTQVLAAD | 229 |
| | PRHPWVESTNYYIKVRAGDNKYMHLKVFNGPWKSHVEVRSADRVLTGYQVD | |
| | KNKDDELTGF | |
| HSA-47 | MIPRGLSEAKPATPEIQEIVDKVKPQLEEKTNETYGKLEAVQYKTQVLAFH | 230 |
| | KRFQSQGSTNYYIKVRAGDNKYMHLKVFNGPWVTQKYIIQADRVLTGYQVD | |
| | KNKDDELTGF | |
| HSA-48 | MIPRGLSEAKPATPEIQEIVDKVKPQLEEKTNETYGKLEAVQYKTQVLAEW | 231 |
| | WQNRWPNSTNYYIKVRAGDNKYMHLKVFNGPWEHAKDWPTADRVLTGYQVD | |
| | KNKDDELTGF | |
| HSA-49 | MIPRGLSEAKPATPEIQEIVDKVKPQLEEKTNETYGKLEAVQYKTQVLAEW | 232 |
| | YQTRWPGSTNYYIKVRAGDNKYMHLKVFNGPFHSKVLDKAADRVLTGYQVD | |
| | KNKDDELTGF | |
| HSA-50 | MIPRGLSEAKPATPEIQEIVDKVKPQLEEKTNETYGKLEAVQYKTQVLAEF | 233 |
| | WQRHWPGSTNYYIKVRAGDNKYMHLKVFNGPYGAQKQAVWADRVLTGYQVD | |
| | KNKDDELTGF | |
| HSA-51 | MIPRGLSEAKPATPEIQEIVDKVKPQLEEKTNETYGKLEAVQYKTQVLAKF | 234 |
| | YERHWPGSTNYYIKVRAGDNKYMHLKVFNGPFSASHFTSQADRVLTGYQVD | |
| | KNKDDELTGF | |
| HSA-41 | MIPRGLSEAKPATPEIQEIVDKVKPQLEEKTNETYGKLEAVQYKTQVLANFF | 235 |
| CQ | QRRWPGSTNYYIKVRAGDNKYMHLKVFNGPWKFRNTDRGADRVLTGYQ | |
| | VDKNKDDELTGFAAAGGRAEQKLISEEDLGCAENLYFQGGAAGHHHHHH | |

In some embodiments, an PD-L1 binding AFFIMER® polypeptide is encoded by a polynucleotide comprising a nucleic acid sequence selected from any one of SEQ ID NOs: 236-243 and 297 (Table 5).

In some embodiments, an PD-L1 binding AFFIMER® polypeptide is encoded by a polynucleotide comprising a nucleic acid sequence having at least 80% or at least 90% identity to the nucleic acid sequence of any one of SEQ ID NOs: 236-243 and 297. In some embodiments, an PD-L1 binding AFFIMER® polypeptide is encoded by a polynucleotide comprising a nucleic acid sequence having 80% to 90% identity to the nucleic acid sequence of any one of SEQ ID NOs: 236-243 and 297. In some embodiments, an PD-L1 binding AFFIMER® polypeptide is encoded by a polynucleotide comprising a nucleic acid sequence of any one of SEQ ID NOs: 236-243 and 297.

In some embodiments, an HSA binding AFFIMER® polypeptide is encoded by a polynucleotide comprising a nucleic acid sequence selected from any one of SEQ ID NOs: 244-276 (Table 6).

In some embodiments, an HSA binding AFFIMER® polypeptide is encoded by a polynucleotide comprising a nucleic acid sequence having at least 80% or at least 90% identity to the nucleic acid sequence of any one of SEQ ID NOs: 244-276. In some embodiments, an HSA binding AFFIMER® polypeptide is encoded by polynucleotide comprising a nucleic acid sequence having 80% to 90% identity to the nucleic acid sequence of any one of SEQ ID NOs: 244-276. In some embodiments, an HSA binding AFFIMER® polypeptide is encoded by a polynucleotide comprising a nucleic acid sequence of any one of SEQ ID NOs: 244-276.

TABLE 5
Examples of PD-L1 Binding AFFIMER ® Polynucleotide Sequences

| Name | DNA sequence | SEQ ID NO: |
|---|---|---|
| PD-L1-00 | ATGATCCCGCGTGGCCTGTCTGAAGCTAAACCAGCAACTCCGGAAATTCAAGAGATC | 297 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAAAGAACATGGTCCAGATTCTTGG | |
| | TGGTCCACCAACTATTACATTAAGGTTCGTGCCGGTGACAATAAGTATATGCACCTG | |
| | AAAGTGTTCAACGGCCCGCAGGAAAAAAACCAGTGGGTTGAAGAAGCGGACCGTGTT | |
| | CTGACCGGTTACCAGGTTGACAAGAACAAAGATGACGAGCTGACGGGTTTC | |
| PD-L1-16 | ATGATCCCGCGTGGCCTGTCTGAAGCTAAACCAGCAACTCCGGAAATTCAAGAGATC | 236 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAAAGATTGGGGTCCATCTAACTGG | |
| | TGGTCCACCAACTATTACATTAAGGTTCGTGCCGGTGACAATAAGTATATGCACCTG | |
| | AAAGTGTTCAACGGCCCGGTTGATGATAAAACCCTGTCTAAAGATGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-05 | ATGATCCCGCGTGGCCTGTCTGAAGCTAAACCAGCAACTCCGGAAATTCAAGAGATC | 237 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAAAGATCATGGTCCAATCGCATGG | |
| | TGGTCCACCAACTATTACATTAAGGTTCGTGCCGGTGACAATAAGTATATGCACCTG | |
| | AAAGTGTTCAACGGCCCGGAAGATACCAACACCGATGGTGCACTGGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-02 | ATGATCCCGCGTGGCCTGTCTGAAGCTAAACCAGCAACTCCGGAAATTCAAGAGATC | 238 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAAACCATACGGTCCACGTGATTGG | |
| | GATTCCACCAACTATTACATTAAGGTTCGTGCCGGTGACAATAAGTATATGCACCTG | |
| | AAAGTGTTCAACGGCCCGGAACCACAGCTGGATACCTCTCCAATCGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-13 | ATGATCCCGCGTGGCCTGTCTGAAGCTAAACCAGCAACTCCGGAAATTCAAGAGATC | 239 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAACACCTGGTTTCCAGAATCTTTT | |
| | TGGTCCACCAACTATTACATTAAGGTTCGTGCCGGTGACAATAAGTATATGCACCTG | |
| | AAAGTGTTCAACGGCCCGGATGATAACCAGGAACGTCAGGAACATGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-34 | ATGATCCCGCGTGGCCTGTCTGAAGCTAAACCAGCAACTCCGGAAATTCAAGAGATC | 240 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCACGTGAAGGTCGTCAGGATTGGGTT | |
| | CTGTCCACCAACTATTACATTAAGGTTCGTGCCGGTGACAATAAGTATATGCACCTG | |
| | AAAGTGTTCAACGGCCCGTGGGTTCCATTTCCACATCAGCAGCTGGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-04 | ATGATCCCGCGTGGCCTGTCTGAAGCTAAACCAGCAACTCCGGAAATTCAAGAGATC | 241 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCACATGCATACGGTCCACGTGATTGG | |
| | GATTCCACCAACTATTACATTAAGGTTCGTGCCGGTGACAATAAGTATATGCACCTG | |
| | AAAGTGTTCAACGGCCCGCCAGCAGATCATGTTCTGGAAGAAGCAGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-03 | ATGATCCCGCGTGGCCTGTCTGAAGCTAAACCAGCAACTCCGGAAATTCAAGAGATC | 242 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAAAGAATACGGTCCAGAAGAATGG | |
| | TGGTCCACCAACTATTACATTAAGGTTCGTGCCGGTGACAATAAGTATATGCACCTG | |
| | AAAGTGTTCAACGGCCCGGGTGATTACGAACAGGTTCTGATCCATGCGGACCGTGTT | |
| | CTGACCGGTTACCAGGTTGACAAGAACAAAGATGACGAGCTGACGGGTTTCGCGGCC | |
| | GCGGGTCATCACCACCACCACCATTAG | |
| PD-L1-33 | ATGATCCCGCGTGGCCTGTCTGAAGCTAAACCAGCAACTCCGGAAATTCAAGAGATC | 243 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGCTGCTCATTTCCCGGAACATTTC | |
| | TGGTCCACCAACTATTACATTAAGGTTCGTGCCGGTGACAATAAGTATATGCACCTG | |
| | AAAGTGTTCAACGGCCCGCAGCCGGCTGATATGTCTGCTGAATTCGCGGACCGTGTT | |
| | CTGACCGGTTACCAGGTTGACAAGAACAAAGATGACGAGCTGACGGGTTTCCTGCAG | |
| | GCGGCCGCGCACCACCACCACCACCACTG | |

TABLE 6
Examples of HSA Binding AFFIMER ® Polynucleotide Sequences

| Name | DNA sequence | SEQ ID NO: |
|---|---|---|
| HSA-18 | ATGATCCCGCGTGGCCTGTCTGAAGCTAAACCAGCAACTCCGGAAATTCAAGAGATC | 244 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGATTGGTGGCAGGCAAAATGGCCA | |
| | CATTCCACCAACTATTACATTAAGGTTCGTGCCGGTGACAATAAGTATATGCACCTG | |
| | AAAGTGTTCAACGGCCCGTACAAAGTTCATCAGTCTTCTGGTGGTGCGGACCGTGTT | |
| | CTGACCGGTTACCAGGTTGACAAGAACAAAGATGACGAGCTGACGGGTTTC | |
| HSA-19 | ATGATCCCGCGTGGCCTGTCTGAAGCTAAACCAGCAACTCCGGAAATTCAAGAGATC | 245 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTATCTGGCAGTCTCGTTGGCCA | |
| | GGTTCCACCAACTATTACATTAAGGTTCGTGCCGGTGACAATAAGTATATGCACCTG | |
| | AAAGTGTTCAACGGCCCGTTTCATCCAATCGCAGGTCGTCCATGGGCGGACCGTGTT | |
| | CTGACCGGTTACCAGGTTGACAAGAACAAAGATGACGAGCTGACGGGTTTC | |
| HSA-20 | ATGATCCCGCGTGGCCTGTCTGAAGCTAAACCAGCAACTCCGGAAATTCAAGAGATC | 246 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTTACTGGGCAGCAAAATGGCCA | |
| | GGTTCCACCAACTATTACATTAAGGTTCGTGCCGGTGACAATAAGTATATGCACCTG | |
| | AAAGTGTTCAACGGCCCGTTTCCAAACACCTCTTACGATCTGCAGGCGGACCGTGTT | |
| | CTGACCGGTTACCAGGTTGACAAGAACAAAGATGACGAGCTGACGGGTTTC | |
| HSA-21 | ATGATCCCGCGTGGCCTGTCTGAAGCTAAACCAGCAACTCCGGAAATTCAAGAGATC | 247 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTTTTTACGCAGATCATTGGCCA | |
| | GGTTCCACCAACTATTACATTAAGGTTCGTGCCGGTGACAATAAGTATATGCACCTG | |
| | AAAGTGTTCAACGGCCCGTTTGCACATTACAACCTGAAATCTGGTGCGGACCGTGTT | |
| | CTGACCGGTTACCAGGTTGACAAGAACAAAGATGACGAGCTGACGGGTTTC | |
| HSA-22 | ATGATCCCGCGTGGCCTGTCTGAAGCTAAACCAGCAACTCCGGAAATTCAAGAGATC | 248 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAACTGGTACCAGCAGCGTTGGCCA | |
| | GGTTCCACCAACTATTACATTAAGGTTCGTGCCGGTGACAATAAGTATATGCACCTG | |
| | AAAGTGTTCAACGGCCCGTGGCATAACTACGGTGAATCTTCTGGTGCGGACCGTGTT | |
| | CTGACCGGTTACCAGGTTGACAAGAACAAAGATGACGAGCTGACGGGTTTC | |
| HSA-23 | ATGATCCCGCGTGGCCTGTCTGAAGCTAAACCAGCAACTCCGGAAATTCAAGAGATC | 249 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTTTTTACGCACGTCATTGGCCA | |
| | GGTTCCACCAACTATTACATTAAGGTTCGTGCCGGTGACAATAAGTATATGCACCTG | |
| | AAAGTGTTCAACGGCCCGAAATTTTACTACGCAGATCATCAGTGGGCGGACCGTGTT | |
| | CTGACCGGTTACCAGGTTGACAAGAACAAAGATGACGAGCTGACGGGTTTC | |
| HSA-24 | ATGATCCCGCGTGGCCTGTCTGAAGCTAAACCAGCAACTCCGGAAATTCAAGAGATC | 250 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGATTTTTGGAAGGCACATTGGCCA | |
| | GGTTCCACCAACTATTACATTAAGGTTCGTGCCGGTGACAATAAGTATATGCACCTG | |
| | AAAGTGTTCAACGGCCCGTACACCCATGCAGATCCACATTCTCAGGCGGACCGTGTT | |
| | CTGACCGGTTACCAGGTTGACAAGAACAAAGATGACGAGCTGACGGGTTTC | |
| HSA-25 | ATGATCCCGCGTGGCCTGTCTGAAGCTAAACCAGCAACTCCGGAAATTCAAGAGATC | 251 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGATTTTTACTCTGTTCGTTGGCCA | |
| | GGTTCCACCAACTATTACATTAAGGTTCGTGCCGGTGACAATAAGTATATGCACCTG | |
| | AAAGTGTTCAACGGCCCGTTTGGTGTTCCACAGCTGGGTGCAGGTGCGGACCGTGTT | |
| | CTGACCGGTTACCAGGTTGACAAGAACAAAGATGACGAGCTGACGGGTTTC | |
| HSA-26 | ATGATCCCGCGTGGCCTGTCTGAAGCTAAACCAGCAACTCCGGAAATTCAAGAGATC | 252 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCATACTGGGCAGCAAACCATGCATCT | |
| | AAATCCACCAACTATTACATTAAGGTTCGTGCCGGTGACAATAAGTATATGCACCTG | |
| | AAAGTGTTCAACGGCCCGTACTCTGGTTTTCCATTTGCAGGTTTTGCGGACCGTGTT | |
| | CTGACCGGTTACCAGGTTGACAAGAACAAAGATGACGAGCTGACGGGTTTC | |
| HSA-27 | ATGATCCCGCGTGGCCTGTCTGAAGCTAAACCAGCAACTCCGGAAATTCAAGAGATC | 253 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAATCAAACGTCTGGAACATTGGGAA | |
| | TACTCCACCAACTATTACATTAAGGTTCGTGCCGGTGACAATAAGTATATGCACCTG | |
| | AAAGTGTTCAACGGCCCGTGGTTTTCTTGGCCATACACCCCACTGGCGGACCGTGTT | |
| | CTGACCGGTTACCAGGTTGACAAGAACAAAGATGACGAGCTGACGGGTTTC | |
| HSA-28 | ATGATCCCGCGTGGCCTGTCTGAAGCTAAACCAGCAACTCCGGAAATTCAAGAGATC | 254 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGAATGGGATTCTCCATGGTCTGAA | |
| | AACTCCACCAACTATTACATTAAGGTTCGTGCCGGTGACAATAAGTATATGCACCTG | |
| | AAAGTGTTCAACGGCCCGTACTACCATCCATCTATCCAGTCTACCGCGGACCGTGTT | |
| | CTGACCGGTTACCAGGTTGACAAGAACAAAGATGACGAGCTGACGGGTTTC | |
| HSA-29 | ATGATCCCGCGTGGCCTGTCTGAAGCTAAACCAGCAACTCCGGAAATTCAAGAGATC | 255 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAAACATAAAAACCTGCGTTGGCCA | |
| | TTTTCCACCAACTATTACATTAAGGTTCGTGCCGGTGACAATAAGTATATGCACCTG | |
| | AAAGTGTTCAACGGCCCGTTTCTGGGTTGGAAAGATACCGTTGTTGCGGACCGTGTT | |
| | CTGACCGGTTACCAGGTTGACAAGAACAAAGATGACGAGCTGACGGGTTTC | |
| HSA-30 | ATGATCCCGCGTGGCCTGTCTGAAGCTAAACCAGCAACTCCGGAAATTCAAGAGATC | 256 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCACGTCATTTTCCAAAACAGACCAAC | |
| | TGGTCCACCAACTATTACATTAAGGTTCGTGCCGGTGACAATAAGTATATGCACCTG | |
| | AAAGTGTTCAACGGCCCGGATTGGTGGAAATGGTGGTGGGCAAAAGCGGACCGTGTT | |
| | CTGACCGGTTACCAGGTTGACAAGAACAAAGATGACGAGCTGACGGGTTTC | |
| HSA-31 | ATGATCCCGCGTGGCCTGTCTGAAGCTAAACCAGCAACTCCGGAAATTCAAGAGATC | 257 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGTTTGGGGTCCAGAATACCAGCAT | |
| | CAGTCCACCAACTATTACATTAAGGTTCGTGCCGGTGACAATAAGTATATGCACCTG | |
| | AAAGTGTTCAACGGCCCGAACGCAGGTTGGCCACTGGTTCCAGAAGCGGACCGTGTT | |
| | CTGACCGGTTACCAGGTTGACAAGAACAAAGATGACGAGCTGACGGGTTTC | |
| HSA-32 | ATGATCCCGCGTGGCCTGTCTGAAGCTAAACCAGCAACTCCGGAAATTCAAGAGATC | 258 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAACCTGGAAAAACAACGGTCAGGAT | |
| | GTTTCCACCAACTATTACATTAAGGTTCGTGCCGGTGACAATAAGTATATGCACCTG | |
| | AAAGTGTTCAACGGCCCGTACGCACTGGATCCATTTGGTGGTAAAGCGGACCGTGTT | |
| | CTGACCGGTTACCAGGTTGACAAGAACAAAGATGACGAGCTGACGGGTTTC | |
| HSA-33 | ATGATCCCGCGTGGCCTGTCTGAAGCTAAACCAGCAACTCCGGAAATTCAAGAGATC | 259 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGCAACCTGGCTGAACTACTACCTG | |
| | CCATCCACCAACTATTACATTAAGGTTCGTGCCGGTGACAATAAGTATATGCACCTG | |
| | AAAGTGTTCAACGGCCCGGGTTACAAATTTTGGGGTGTTTCTGATGCGGACCGTGTT | |
| | CTGACCGGTTACCAGGTTGACAAGAACAAAGATGACGAGCTGACGGGTTTC | |
| HSA-34 | ATGATCCCGCGTGGCCTGTCTGAAGCTAAACCAGCAACTCCGGAAATTCAAGAGATC | 260 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGATCAGGAATCTCTGTTTCTGAAC | |
| | AACTCCACCAACTATTACATTAAGGTTCGTGCCGGTGACAATAAGTATATGCACCTG | |
| | AAAGTGTTCAACGGCCCGCAGGGTAAACAGTACATCCTGCTGCGTGCGGACCGTGTT | |
| | CTGACCGGTTACCAGGTTGACAAGAACAAAGATGACGAGCTGACGGGTTTC | |
| HSA-35 | ATGATCCCGCGTGGCCTGTCTGAAGCTAAACCAGCAACTCCGGAAATTCAAGAGATC | 261 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTTTTTACGCACAGCATTGGCCA | |
| | GATTCCACCAACTATTACATTAAGGTTCGTGCCGGTGACAATAAGTATATGCACCTG | |
| | AAAGTGTTCAACGGCCCGTACAAACGTCATTCTGCACATGATTACGCGGACCGTGTT | |
| | CTGACCGGTTACCAGGTTGACAAGAACAAAGATGACGAGCTGACGGGTTTC | |
| HSA-36 | ATGATCCCGCGTGGCCTGTCTGAAGCTAAACCAGCAACTCCGGAAATTCAAGAGATC | 262 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTCATTACGCACGTTACTGGCCA | |
| | GGTTCCACCAACTATTACATTAAGGTTCGTGCCGGTGACAATAAGTATATGCACCTG | |
| | AAAGTGTTCAACGGCCCGTGGGCACAGAAATCTAAAGTTCATCAGGCGGACCGTGTT | |
| | CTGACCGGTTACCAGGTTGACAAGAACAAAGATGACGAGCTGACGGGTTTC | |
| HSA-37 | ATGATCCCGCGTGGCCTGTCTGAAGCTAAACCAGCAACTCCGGAAATTCAAGAGATC | 263 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTTTTTGGGCAAGTAAATGGCCA | |
| | GGTTCCACCAACTATTACATTAAGGTTCGTGCCGGTGACAATAAGTATATGCACCTG | |
| | AAAGTGTTCAACGGCCCGTTTACCGCAGTTTCTAAAAAAGATGCAGCGGACCGTGTT | |
| | CTGACCGGTTACCAGGTTGACAAGAACAAAGATGACGAGCTGACGGGTTTC | |
| HSA-38 | ATGATCCCGCGTGGCCTGTCTGAAGCTAAACCAGCAACTCCGGAAATTCAAGAGATC | 264 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTTTTTGGCAGCGTAAATGGCCA | |
| | AACTCCACCAACTATTACATTAAGGTTCGTGCCGGTGACAATAAGTATATGCACCTG | |
| | AAAGTGTTCAACGGCCCGTGGGGTGATAAAGAAAACATCTGGTTTGCGGACCGTGTT | |
| | CTGACCGGTTACCAGGTTGACAAGAACAAAGATGACGAGCTGACGGGTTTC | |
| HSA-39 | ATGATCCCGCGTGGCCTGTCTGAAGCTAAACCAGCAACTCCGGAAATTCAAGAGATC | 265 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGTTTGGCCAGCAGATAACGATCTG | |
| | AAATCCACCAACTATTACATTAAGGTTCGTGCCGGTGACAATAAGTATATGCACCTG | |
| | AAAGTGTTCAACGGCCCGTGGTCTGGTCATCCATGGGTTCAGAAAGCGGACCGTGTT | |
| | CTGACCGGTTACCAGGTTGACAAGAACAAAGATGACGAGCTGACGGGTTTC | |
| HSA-40 | ATGATCCCGCGTGGCCTGTCTGAAGCTAAACCAGCAACTCCGGAAATTCAAGAGATC | 266 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCACATTGGGCATGGACCTCTCCAGGT | |
| | TACTCCACCAACTATTACATTAAGGTTCGTGCCGGTGACAATAAGTATATGCACCTG | |
| | AAAGTGTTCAACGGCCCGTACGCAGATTACCCACTGTCTCCAAAAGCGGACCGTGTT | |
| | CTGACCGGTTACCAGGTTGACAAGAACAAAGATGACGAGCTGACGGGTTTC | |
| HSA-41 | ATGATCCCGCGTGGCCTGTCTGAAGCTAAACCAGCAACTCCGGAAATTCAAGAGATC | 267 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAAACTTTTTTCAGCGTCGTTGGCCA | |
| | GGTTCCACCAACTATTACATTAAGGTTCGTGCCGGTGACAATAAGTATATGCACCTG | |
| | AAAGTGTTCAACGGCCCGTGGAAATTTCGTAACACCGATCGTGGTGCGGACCGTGTT | |
| | CTGACCGGTTACCAGGTTGACAAGAACAAAGATGACGAGCTGACGGGTTTC | |
| HSA-42 | ATGATCCCGCGTGGCCTGTCTGAAGCTAAACCAGCAACTCCGGAAATTCAAGAGATC | 268 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCACATCATTCTCATCGTCTGAAAGGT | |
| | CAGTCCACCAACTATTACATTAAGGTTCGTGCCGGTGACAATAAGTATATGCACCTG | |
| | AAAGTGTTCAACGGCCCGCAGACCGTTGCAACCCATTACCATTACGCGGACCGTGTT | |
| | CTGACCGGTTACCAGGTTGACAAGAACAAAGATGACGAGCTGACGGGTTTC | |
| HSA-43 | ATGATCCCGCGTGGCCTGTCTGAAGCTAAACCAGCAACTCCGGAAATTCAAGAGATC | 269 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCATACCAGAACACCATCTTTCTGTCT | |
| | ATCTCCACCAACTATTACATTAAGGTTCGTGCCGGTGACAATAAGTATATGCACCTG | |
| | AAAGTGTTCAACGGCCCGTGGCATGCAAAACATCTGCTGTCTCATGCGGACCGTGTT | |
| | CTGACCGGTTACCAGGTTGACAAGAACAAAGATGACGAGCTGACGGGTTTC | |
| HSA-45 | ATGATCCCGCGTGGCCTGTCTGAAGCTAAACCAGCAACTCCGGAAATTCAAGAGATC | 270 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCATTTCAGGATCAGTTTACCTGGTCT | |
| | CAGTCCACCAACTATTACATTAAGGTTCGTGCCGGTGACAATAAGTATATGCACCTG | |
| | AAAGTGTTCAACGGCCCGTCTGGTATCAAAAAAGCAGATTCTGTTGCGGACCGTGTT | |
| | CTGACCGGTTACCAGGTTGACAAGAACAAAGATGACGAGCTGACGGGTTTC | |
| HSA-46 | ATGATCCCGCGTGGCCTGTCTGAAGCTAAACCAGCAACTCCGGAAATTCAAGAGATC | 271 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGGTGAACCACATTGGCCATGGCAG | |
| | GCATCCACCAACTATTACATTAAGGTTCGTGCCGGTGACAATAAGTATATGCACCTG | |
| | AAAGTGTTCAACGGCCCGAAAGCAAATTTGATAAACGTGAAATCTGCGGACCGTGTT | |
| | CTGACCGGTTACCAGGTTGACAAGAACAAAGATGACGAGCTGACGGGTTTC | |
| HSA-47 | ATGATCCCGCGTGGCCTGTCTGAAGCTAAACCAGCAACTCCGGAAATTCAAGAGATC | 272 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGCAGATCCACGTCATCCATGGGTT | |
| | GAATCCACCAACTATTACATTAAGGTTCGTGCCGGTGACAATAAGTATATGCACCTG | |
| | AAAGTGTTCAACGGCCCGTGGAAATCTCATGTTGAAGTTCGTTCTGCGGACCGTGTT | |
| | CTGACCGGTTACCAGGTTGACAAGAACAAAGATGACGAGCTGACGGGTTTC | |
| HSA-48 | ATGATCCCGCGTGGCCTGTCTGAAGCTAAACCAGCAACTCCGGAAATTCAAGAGATC | 273 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCATTTCATAAACGTTTTCAGTCTCAG | |
| | GGTTCCACCAACTATTACATTAAGGTTCGTGCCGGTGACAATAAGTATATGCACCTG | |
| | AAAGTGTTCAACGGCCCGTGGGTTACCCAGAAATACATCATCCAGGCGGACCGTGTT | |
| | CTGACCGGTTACCAGGTTGACAAGAACAAAGATGACGAGCTGACGGGTTTC | |
| HSA-49 | ATGATCCCGCGTGGCCTGTCTGAAGCTAAACCAGCAACTCCGGAAATTCAAGAGATC | 274 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGAATGGTGGCAGAACCGTTGGCCA | |
| | AACTCCACCAACTATTACATTAAGGTTCGTGCCGGTGACAATAAGTATATGCACCTG | |
| | AAAGTGTTCAACGGCCCGTGGGAACATGCAAAAGATTGGCCAACCGCGGACCGTGTT | |
| | CTGACCGGTTACCAGGTTGACAAGAACAAAGATGACGAGCTGACGGGTTTC | |
| HSA-50 | ATGATCCCGCGTGGCCTGTCTGAAGCTAAACCAGCAACTCCGGAAATTCAAGAGATC | 275 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGAATGGTACCAGACCCGTTGGCCA | |
| | GGTTCCACCAACTATTACATTAAGGTTCGTGCCGGTGACAATAAGTATATGCACCTG | |
| | AAAGTGTTCAACGGCCCGTTTCATTCTAAAGTTCTGGATAAAGCAGCGGACCGTGTT | |
| | CTGACCGGTTACCAGGTTGACAAGAACAAAGATGACGAGCTGACGGGTTTC | |
| HSA-51 | ATGATCCCGCGTGGCCTGTCTGAAGCTAAACCAGCAACTCCGGAAATTCAAGAGATC | 276 |
| | GTCGATAAGGTGAAACCGCAGCTGGAAGAGAAAACGAACGAAACCTACGGTAAGCTG | |
| | GAAGCGGTCCAGTACAAAACCCAAGTGCTAGCAGAATTTTGGCAGCGTCATTGGCCA | |
| | GGTTCCACCAACTATTACATTAAGGTTCGTGCCGGTGACAATAAGTATATGCACCTG | |
| | AAAGTGTTCAACGGCCCGTACGGTGCACAGAAACAGGCAGTTTGGGCGGACCGTGTT | |
| | CTGACCGGTTACCAGGTTGACAAGAACAAAGATGACGAGCTGACGGGTTTC | |

The fusion proteins here may include any one or more of the PD-L1 binding AFFIMER® polypeptides and/or any one or more of the HSA binding AFFIMER® polypeptides. For example, a fusion protein may compress one, two, three or more PD-L1 binding AFFIMER® polypeptide molecules and one, two, three or more PD-L1 binding AFFIMER® polypeptide molecules. In some embodiments, a fusion protein comprises three (at least three) PD-L1 binding AFFIMER® polypeptide molecules and one (at least one) HSA binding AFFIMER® polypeptide molecules.

In some embodiments, a fusion protein comprises two PD-L1 binding AFFIMER® polypeptides and one HSA binding AFFIMER® polypeptide. In some embodiments, the fusion protein comprises, from N-terminal to C-terminal, a first PDL-L1 binding AFFIMER® polypeptide, a second PD-L1 binding AFFIMER® polypeptide, and an HSA binding AFFIMER® polypeptide. In some embodiments, the fusion protein comprises, from N-terminal to C-terminal, a first PDL-L1 binding AFFIMER® polypeptide, an HSA binding AFFIMER® polypeptide, and a second PD-L1 binding AFFIMER® polypeptide. In some embodiments, the fusion protein comprises, from N-terminal to C-terminal, an HSA binding AFFIMER® polypeptide, a first PDL-L1 binding AFFIMER® polypeptide, and a second PD-L1 binding AFFIMER® polypeptide. In some embodiments, the AFFIMER® polypeptides of the fusion protein are directly conjugated to one another. In some embodiments, the AFFIMER® polypeptides of the fusion protein are conjugated to one another via one or more linkers, as described herein.

In some embodiments, an AFFIMER® fusion protein comprises an amino acid sequence having at least 80% or at least 90% identity to any one of the amino acid sequences in Table 7 (e.g., SEQ ID NOs: 277-291). In some embodiments, an AFFIMER® fusion protein comprises an amino acid sequence having 80% to 90% identity to any one of the amino acid sequences of SEQ ID NO: 277-291. In some embodiments, an AFFIMER® fusion protein comprises the any one of the amino acid sequences of SEQ ID NO: 277-291.

In some embodiments, an AFFIMER® fusion protein comprises an amino acid sequence having at least 80%, at least 85%, at least 90%, at least 95%, or 100% identity to SEQ ID NO: 277. In some embodiments, an AFFIMER® fusion protein comprises an amino acid sequence having at least 80%, at least 85%, at least 90%, at least 95%, or 100% identity to SEQ ID NO: 278. In some embodiments, an AFFIMER® fusion protein comprises an amino acid sequence having at least 80%, at least 85%, at least 90%, at least 95%, or 100% identity to SEQ ID NO: 279. In some embodiments, an AFFIMER® fusion protein comprises an amino acid sequence having at least 80%, at least 85%, at least 90%, at least 95%, or 100% identity to SEQ ID NO: 280. In some embodiments, an AFFIMER® fusion protein comprises an amino acid sequence having at least 80%, at least 85%, at least 90%, at least 95%, or 100% identity to SEQ ID NO: 281. In some embodiments, an AFFIMER® fusion protein comprises an amino acid sequence having at least 80%, at least 85%, at least 90%, at least 95%, or 100% identity to SEQ ID NO: 282. In some embodiments, an AFFIMER® fusion protein comprises an amino acid sequence having at least 80%, at least 85%, at least 90%, at least 95%, or 100% identity to SEQ ID NO: 283. In some embodiments, an AFFIMER® fusion protein comprises an amino acid sequence having at least 80%, at least 85%, at least 90%, at least 95%, or 100% identity to SEQ ID NO: 284. In some embodiments, an AFFIMER® fusion protein comprises an amino acid sequence having at least 80%, at least 85%, at least 90%, at least 95%, or 100% identity to SEQ ID NO: 285. In some embodiments, an AFFIMER® fusion protein comprises an amino acid sequence having at least 80%, at least 85%, at least 90%, at least 95%, or 100% identity to SEQ ID NO: 286. In some embodiments, an AFFIMER® fusion protein comprises an amino acid sequence having at least 80%, at least 85%, at least 90%, at least 95%, or 100% identity to SEQ ID NO: 287. In some embodiments, an AFFIMER® fusion protein comprises an amino acid sequence having at least 80%, at least 85%, at least 90%, at least 95%, or 100% identity to SEQ ID NO: 288. In some embodiments, an AFFIMER® fusion protein comprises an amino acid sequence having at least 80%, at least 85%, at least 90%, at least 95%, or 100% identity to SEQ ID NO: 289. In some embodiments, an AFFIMER® fusion protein comprises an amino acid sequence having at least 80%, at least 85%, at least 90%, at least 95%, or 100% identity to SEQ ID NO: 290. In some embodiments, an AFFIMER® fusion protein comprises an amino acid sequence having at least 80%, at least 85%, at least 90%, at least 95%, or 100% identity to SEQ ID NO: 291.

TABLE 7
Half-life extension in-line fusions with PD-L1 binding AFFIMER ® proteins

| | | SEQ ID |
|---|---|---|
| Name | Sequence | NO: |
| AVA04-236 | MIPRGLSEAKPATPEIQEIVDKVKPQLEEKTNETYGKLEAVQYKTQVLAKEHGPD | 277 |
| XT7 | SWWSTNYYIKVRAGDNKYMHLKVFNGPQEKNQWVEEADRVLTGYQVDKNKDDELT | |
| | GFGGGGSGGGGSGGGGSGGGGSGGGGSGGGGSMIPRGLSEAKPATPEIQEIVDKV | |
| | KPQLEEKTNETYGKLEAVQYKTQVLANFFQRRWPGSTNYYIKVRAGDNKYMHLKV | |
| | FNGPWKFRNTDRGADRVLTGYQVDKNKDDELTGF | |
| AVA04-236 | MIPRGLSEAKPATPEIQEIVDKVKPQLEEKTNETYGKLEAVQYKTQVLAKEHGPD | 278 |
| XT8 | SWWSTNYYIKVRAGDNKYMHLKVFNGPQEKNQWVEEADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLANFFQRRWPGSTNYYIKVRAGDNKYMHLK | |
| | VFNGPWKFRNTDRGADRVLTGYQVDKNKDDELTGF | |
| AVA04-261 | MIPRGLSEAKPATPEIQEIVDKVKPQLEEKTNETYGKLEAVQYKTQVLAHAYGPR | 279 |
| XT9 | DWDSTNYYIKVRAGDNKYMHLKVFNGPPADHVLEEAADRVLTGYQVDKNKDDELT | |
| | GFGGGGSGGGGSGGGGSGGGGSGGGGSGGGGSMIPRGLSEAKPATPEIQEIVDKV | |
| | KPQLEEKTNETYGKLEAVQYKTQVLANFFQRRWPGSTNYYIKVRAGDNKYMHLKV | |
| | FNGPWKFRNTDRGADRVLTGYQVDKNKDDELTGF | |
| AVA04-261 | MIPRGLSEAKPATPEIQEIVDKVKPQLEEKTNETYGKLEAVQYKTQVLAHAYGPR | 280 |
| XT10 | DWDSTNYYIKVRAGDNKYMHLKVFNGPPADHVLEEAADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLANFFQRRWPGSTNYYIKVRAGDNKYMHLK | |
| | VFNGPWKFRNTDRGADRVLTGYQVDKNKDDELTGF | |
| AVA04-269 | MIPRGLSEAKPATPEIQEIVDKVKPQLEEKTNETYGKLEAVQYKTQVLAKEYGPE | 281 |
| XT11 | EWWSTNYYIKVRAGDNKYMHLKVFNGPGDYEQVLIHADRVLTGYQVDKNKDDELT | |
| | GFGGGGSGGGGSGGGGSGGGGSGGGGSGGGGSMIPRGLSEAKPATPEIQEIVDKV | |
| | KPQLEEKTNETYGKLEAVQYKTQVLANFFQRRWPGSTNYYIKVRAGDNKYMHLKV | |
| | FNGPWKFRNTDRGADRVLTGYQVDKNKDDELTGF | |
| AVA04-269 | MIPRGLSEAKPATPEIQEIVDKVKPQLEEKTNETYGKLEAVQYKTQVLAKEYGPE | 282 |
| XT12 | EWWSTNYYIKVRAGDNKYMHLKVFNGPGDYEQVLIHADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLANFFQRRWPGSTNYYIKVRAGDNKYMHLK | |
| | VFNGPWKFRNTDRGADRVLTGYQVDKNKDDELTGF | |
| AVA04-251 | MIPRGLSEAKPATPEIQEIVDKVKPQLEEKTNETYGKLEAVQYKTQVLAREGRQD | 283 |
| XT14 | WVLSTNYYIKVRAGDNKYMHLKVFNGPWVPFPHQQLADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLAREGRQDWVLSTNYYIKVRAGDNKYMHLK | |
| | VFNGPWVPFPHQQLADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLANFFQRRWPGSTNYYIKVRAGDNKYMHLKVFNGPWKFRNTDRGADRVLTGY | |
| | QVDKNKDDELTGF | |
| AVA04-251 | MIPRGLSEAKPATPEIQEIVDKVKPQLEEKTNETYGKLEAVQYKTQVLAREGRQD | 284 |
| XT15 | WVLSTNYYIKVRAGDNKYMHLKVFNGPWVPFPHQQLADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLANFFQRRWPGSTNYYIKVRAGDNKYMHLK | |
| | VFNGPWKFRNTDRGADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLAREGRQDWVLSTNYYIKVRAGDNKYMHLKVFNGPWVPFPHQQLADRVLTGY | |
| | QVDKNKDDELTGF | |
| AVA04-251 | MIPRGLSEAKPATPEIQEIVDKVKPQLEEKTNETYGKLEAVQYKTQVLANFFQRR | 285 |
| XT16 | WPGSTNYYIKVRAGDNKYMHLKVFNGPWKFRNTDRGADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLAREGRQDWVLSTNYYIKVRAGDNKYMHLK | |
| | VFNGPWVPFPHQQLADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLAREGRQDWVLSTNYYIKVRAGDNKYMHLKVFNGPWVPFPHQQLADRVLTGY | |
| | QVDKNKDDELTGF | |
| AVA04-251 | MIPRGLSEAKPATPEIQEIVDKVKPQLEEKTNETYGKLEAVQYKTQVLAREGRQD | 286 |
| XT14 cys | WVLSTNYYIKVRAGDNKYMHLKVFNGPWVPFPHQQLADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLAREGRQDWVLSTNYYIKVRAGDNKYMHLK | |
| | VFNGPWVPFPHQQLADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLANFFQRRWPGSTNYYIKVRAGDNKYMHLKVFNGPWKFRNTDRGADRVLTGY | |
| | QVDKNKDDELTGFC | |
| AVA04-182 | MIPRGLSEAKPATPEIQEIVDKVKPQLEEKTNETYGKLEAVQYKTQVLAFALPEF | 287 |
| XT20 | EYMSTNYYIKVRAGDNKYMHLKVFNGPPMIRRKNEVADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLAFALPEFEYMSTNYYIKVRAGDNKYMHLK | |
| | VFNGPPMIRRKNEVADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLANFFQRRWPGSTNYYIKVRAGDNKYMHLKVFNGPWKFRNTDRGADRVLTGY | |
| | QVDKNKDDELTGF | |
| SQT gly XT28 | MIPRGLSEAKPATPEIQEIVDKVKPQLEEKTNETYGKLEAVQYKTQVLAGGGGGG | 288 |
| | GGGSTNYYIKVRAGDNKYMHLKVFNGPGGGGGGGGGADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLAGGGGGGGGGSTNYYIKVRAGDNKYMHLK | |
| | VFNGPGGGGGGGGGADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLANFFQRRWPGSTNYYIKVRAGDNKYMHLKVFNGPWKFRNTDRGADRVLTGY | |
| | QVDKNKDDELTGF | |
| AFFIMER ® | MIPRGLSEAKPATPEIQEIVDKVKPQLEEKTNETYGKLEAVQYKTQVLANFFQRR | 289 |
| XT | WPGSTNYYIKVRAGDNKYMHLKVFNGPWKFRNTDRGADRVLTGYQVDKNKDDELT | |
| “AVA-41” | GF | |
| AVA04-251 | MIPRGLSEAKPATPEIQEIVDKVKPQLEEKTNETYGKLEAVQYKTQVLAREGRQD | 290 |
| XT60 | WVLSTNYYIKVRAGDNKYMHLKVFNGPWVPFPHQQLADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLAREGRQDWVLSTNYYIKVRAGDNKYMHLK | |
| | VFNGPWVPFPHQQLADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLADWWQAKWPHSTNYYIKVRAGDNKYMHLKVFNGPYKVHQSSGGADRVLTGY | |
| | QVDKNKDDELTGF | |
| AVA04-251 | MIPRGLSEAKPATPEIQEIVDKVKPQLEEKTNETYGKLEAVQYKTQVLAREGRQD | 291 |
| XT61 | WVLSTNYYIKVRAGDNKYMHLKVFNGPWVPFPHQQLADRVLTGYQVDKNKDDELT | |
| | GFAEAAAKEAAAKEAAAKEAAAKEAAAKEAAAKMIPRGLSEAKPATPEIQEIVDK | |
| | VKPQLEEKTNETYGKLEAVQYKTQVLADWWQAKWPHSTNYYIKVRAGDNKYMHLK | |
| | VFNGPYKVHQSSGGADRVLTGYQVDKNKDDELTGFAEAAAKEAAAKEAAAKEAAA | |
| | KEAAAKEAAAKMIPRGLSEAKPATPEIQEIVDKVKPQLEEKTNETYGKLEAVQYK | |
| | TQVLAREGRQDWVLSTNYYIKVRAGDNKYMHLKVFNGPWVPFPHQQLADRVLTGY | |
| | QVDKNKDDELTGF | |

The fusion proteins provided herein include an HSA binding AFFIMER® polypeptide linked to a PD-L1 binding AFFIMER® polypeptide and has an extended half-life due to the presence of the binding AFFIMER® polypeptide. The term half-life refers to the amount of time it takes for a substance (e.g., a protein comprising a PD-L1 binding AFFIMER® polypeptide) to lose half of its pharmacologic or physiologic activity or concentration. Biological half-life can be affected by elimination, excretion, degradation (e.g., enzymatic degradation) of the substance, or absorption and concentration in certain organs or tissues of the body. Biological half-life can be assessed, for example, by determining the time it takes for the blood plasma concentration of the substance to reach half its steady state level (“plasma half-life”).

In some embodiments, an HSA binding AFFIMER® polypeptide extends the serum half-life of the PD-L1 binding AFFIMER® polypeptide in vivo. For example, an HSA binding AFFIMER® polypeptide may extend the half-life of the PD-L1 binding AFFIMER® polypeptide by at least 1.2-fold, relative to the half-life of the PD-L1 binding AFFIMER® polypeptide not linked to an HSA binding AFFIMER® polypeptide. In some embodiments, an HSA binding AFFIMER® polypeptide extends the half-life of the PD-L1 binding AFFIMER® polypeptide by at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, or at least 30-fold, relative to the half-life of the PD-L1 binding AFFIMER® polypeptide not linked to an HSA binding AFFIMER® polypeptide. In some embodiments, an HSA binding AFFIMER® polypeptide extends the half-life of the PD-L1 binding AFFIMER® polypeptide by 1.2-fold to 5-fold, 1.2-fold to 10-fold, 1.5-fold to 5-fold, 1.5-fold to 10-fold, 2-fold to 5-fold, 2-fold to 10-fold, 3-fold to 5-fold, 3-fold to 10-fold, 15-fold to 5-fold, 4-fold to 10-fold, or 5-fold to 10-fold, relative to the half-life of the PD-L1 binding AFFIMER® polypeptide not linked to an HSA binding AFFIMER® polypeptide. In some embodiments, an HSA binding AFFIMER® polypeptide extends the half-life of the PD-L1 binding AFFIMER® polypeptide by at least 6 hours, at least 12 hours, at least 24 hours, at least 48 hours, at least 72 hours, at least 96 hours, for example, at least 1 week after in vivo administration, relative to the half-life of the PD-L1 binding AFFIMER® polypeptide not linked to an HSA binding AFFIMER® polypeptide.

### Polypeptides

A polypeptide is a polymer of amino acids (naturally-occurring or non-naturally occurring, e.g., amino acid analogs) of any length. The terms “polypeptide” and “peptide” are used interchangeably herein unless noted otherwise. A protein is one example of a polypeptide. It should be understood that a polypeptide may be linear or branched, it may comprise naturally-occurring and/or non-naturally-occurring (e.g., modified) amino acids, and/or it may include non-amino acids (e.g., interspersed throughout the polymer). A polypeptide, as provided herein, may be modified (e.g., naturally or non-naturally), for example, via disulfide bond formation, glycosylation, lipidation, acetylation, phosphorylation, or conjugation with a labeling component. Polypeptides, in some instances, may contain at least one analog of an amino acid (including, for example, unnatural amino acids) and/or other modifications.

An amino acid (also referred to as an amino acid residue) participates in peptide bonds of a polypeptide. In general, the abbreviations used herein for designating the amino acids are based on recommendations of the IUPAC-IUB Commission on Biochemical Nomenclature (see Biochemistry (1972) 11:1726-1732). For instance, Met, Ile, Leu, Ala and Gly represent “residues” of methionine, isoleucine, leucine, alanine and glycine, respectively. A residue is a radical derived from the corresponding α-amino acid by eliminating the OH portion of the carboxyl group and the H portion of the α-amino group. An amino acid side chain is that part of an amino acid exclusive of the —CH(NH2)COOH portion, as defined by K. D. Kopple, “Peptides and Amino Acids”, W. A. Benjamin Inc., New York and Amsterdam, 1966, pages 2 and 33.

Amino acids used herein, in some embodiments, are naturally-occurring amino acids found in proteins, for example, or the naturally-occurring anabolic or catabolic products of such amino acids that contain amino and carboxyl groups. Examples of amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs that have been identified as constituents of peptidylglycan bacterial cell walls.

Amino acids having basic sidechains include Arg, Lys and His. Amino acids having acidic sidechains include Glu and Asp. Amino acids having neutral polar sidechains include Ser, Thr, Asn, Gln, Cys and Tyr. Amino acids having neutral non-polar sidechains include Gly, Ala, Val, Ile, Leu, Met, Pro, Trp and Phe. Amino acids having non-polar aliphatic sidechains include Gly, Ala, Val, Ile and Leu. Amino acids having hydrophobic sidechains include Ala, Val, Ile, Leu, Met, Phe, Tyr and Trp. Amino acids having small hydrophobic sidechains include Ala and Val. Amino acids having aromatic sidechains include Tyr, Trp and Phe.

The term amino acid includes analogs, derivatives and congeners of any specific amino acid referred to herein; for instance, the AFFIMER® polypeptides (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally-occurring amino acid metabolites or precursors having side chains that are suitable herein will be recognized by those skilled in the art and are included in the scope of the present disclosure.

Also included herein are the (D) and (L) stereoisomers of such amino acids when the structure of the amino acid admits of stereoisomeric forms. The configuration of the amino acids and amino acids herein are designated by the appropriate symbols (D), (L) or (DL); furthermore, when the configuration is not designated the amino acid or residue can have the configuration (D), (L) or (DL). It will be noted that the structure of some of the compounds of the present disclosure includes asymmetric carbon atoms. It is to be understood accordingly that the isomers arising from such asymmetry are included within the scope of the present disclosure. Such isomers can be obtained in substantially pure form by classical separation techniques and by sterically controlled synthesis. For the purposes of this disclosure, unless expressly noted to the contrary, a named amino acid shall be construed to include both the (D) or (L) stereoisomers.

Percent identity, in the context of two or more nucleic acids or polypeptides, refers to two or more sequences or subsequences that are the same (identical/100% identity) or have a specified percentage (e.g., at least 70% identity) of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the present disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A conservative amino acid substitution is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the present disclosure do not abrogate the binding of the polypeptide, soluble protein, or antibody containing the amino acid sequence, to the target binding site. Methods of identifying amino acid conservative substitutions that do not eliminate binding are well-known in the art.

Herein, it should be understood that an isolated molecule (e.g., polypeptide (e.g., soluble protein, antibody, etc.), polynucleotide (e.g., vector), cell, or other composition) is in a form not found in nature. Isolated molecules, for example, have been purified to a degree that is not possible in nature.

In some embodiments, an isolated molecule (e.g., polypeptide (e.g., soluble protein, antibody, etc.), polynucleotide (e.g., vector), cell, or other composition) is substantially pure, which refer to an isolated molecule that is at least 50% pure (e.g., free from 50% of contaminants associated with the unpurified form of the molecule), at least 90% pure, at least 95% pure, at least 98% pure, or at least 99% pure.

Conjugates, Including Polypeptide Fusions

The verb conjugate (used interchangeably with the verb link) herein refers to the joining together of two or more molecules (e.g., polypeptides and/or chemical moieties) to form another molecule. Thus, one molecule (e.g., a PD-L1 binding AFFIMER® polypeptide) conjugated to another molecule (e.g., a PD-L1 AFFIMER® polypeptide, drug molecule, or other therapeutic protein or nucleic acid) forms a conjugate. The joining of two or more molecules can be, for example, through a non-covalent bond or a covalent bond. Non-limiting examples of conjugates include chemical conjugates (e.g., joined through “click” chemistry or another chemical reaction) and fusions (two molecules linked by contiguous peptide bonds). In some embodiments, a conjugate is a fusion polypeptide, for example, a fusion protein.

A fusion polypeptide (e.g., fusion protein) is a polypeptide comprising at least two domains (e.g., protein domains) encoded by a polynucleotide comprising nucleotide sequences of at least two separate molecules (e.g., two genes). In some embodiments, a fusion protein comprises two AFFIMER® polypeptides covalently linked (to an amino acid of the polypeptide) through an amide bond to form a contiguous fusion polypeptide (e.g., fusion protein). In some embodiments, a fusion protein comprises three AFFIMER® polypeptides covalently linked (to an amino acid of the polypeptide) through an amide bond to form a contiguous fusion polypeptide (e.g., fusion protein). In some embodiments, AFFIMER® polypeptides (e.g., 2, 3, 4, or more AFFIMER® polypeptides) are conjugated to each other through contiguous peptide bonds at the C-terminus or N-terminus of the AFFIMER® polypeptide (e.g., an HSA binding AFFIMER® polypeptide).

A linker is a molecule inserted between a first polypeptide (e.g., an AFFIMER® polypeptide) and a second polypeptide (e.g., another AFFIMER® polypeptide, an Fc domain, a ligand binding domain, etc.). A linker may be any molecule, for example, one or more nucleotides, amino acids, chemical functional groups. In some embodiments, the linker is a peptide linker (e.g., two or more amino acids). Linkers should not adversely affect the expression, secretion, or bioactivity of the polypeptides. In some embodiments, linkers are not antigenic and do not elicit an immune response. An immune response includes a response from the innate immune system and/or the adaptive immune system. Thus, an immune response may be a cell-mediate response and/or a humoral immune response. The immune response may be, for example, a T cell response, a B cell response, a natural killer (NK) cell response, a monocyte response, and/or a macrophage response. Other cell responses are contemplated herein.

In some embodiments, linkers are non-protein-coding.

Empirical linkers designed by researchers are generally classified into 3 categories according to their structures: flexible linkers, rigid linkers, and in vivo cleavable linkers. Besides the basic role in linking the functional domains together (as in flexible and rigid linkers) or releasing free functional domain in vivo (as in in vivo cleavable linkers), linkers may offer many other advantages for the production of fusion proteins, such as improving biological activity, increasing expression yield, and achieving desirable pharmacokinetic profiles. Linkers should not adversely affect the expression, secretion, or bioactivity of the fusion protein. Linkers should not be antigenic and should not elicit an immune response.

Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. In some embodiments, the linker may comprise a cleavage site. In some embodiments, the linker may comprise an enzyme cleavage site, so that the second polypeptide may be separated from the first polypeptide.

In some embodiments, the linker can be characterized as flexible. Flexible linkers are usually applied when the joined domains require a certain degree of movement or interaction. They are generally composed of small, non-polar (e.g. Gly) or polar (e.g. Ser or Thr) amino acids. See, for example, Argos P. (1990) “An investigation of oligopeptides linking domains in protein tertiary structures and possible candidates for general gene fusion” J Mol Biol. 211:943-958. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). An example of the most widely used flexible linker has the sequence of (Gly-Gly-Gly-Gly-Ser)n. By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. As These flexible linkers are also rich in small or polar amino acids such as Gly and Ser but can contain additional amino acids such as Thr and Ala to maintain flexibility, as well as polar amino acids such as Lys and Glu to improve solubility.

In some embodiments, the linker can be characterized as rigid. While flexible linkers have the advantage to connect the functional domains passively and permitting certain degree of movements, the lack of rigidity of these linkers can be a limitation in certain fusion protein embodiments, such as in expression yield or biological activity. The ineffectiveness of flexible linkers in these instances was attributed to an inefficient separation of the protein domains or insufficient reduction of their interference with each other. Under these situations, rigid linkers have been successfully applied to keep a fixed distance between the domains and to maintain their independent functions.

Many natural linkers exhibited α-helical structures. The α-helical structure was rigid and stable, with intra-segment hydrogen bonds and a closely packed backbone. Therefore, the stiff α-helical linkers can act as rigid spacers between protein domains. George et al. (2002) “An analysis of protein domain linkers: their classification and role in protein folding” Protein Eng. 15(11):871-9. In general, rigid linkers exhibit relatively stiff structures by adopting α-helical structures or by containing multiple Pro residues. Under many circumstances, they separate the functional domains more efficiently than the flexible linkers. The length of the linkers can be easily adjusted by changing the copy number to achieve an optimal distance between domains. As a result, rigid linkers are chosen when the spatial separation of the domains is critical to preserve the stability or bioactivity of the fusion proteins. In this regard, alpha helix-forming linkers with the sequence of (EAAAK)n have been applied to the construction of many recombinant fusion proteins. Another type of rigid linkers has a Pro-rich sequence, (XP)n, with X designating any amino acid, preferably Ala, Lys, or Glu.

Merely to illustrate, exemplary linkers include:

| | | |
|---|---|---|
| Flexible | (GGGGS)ₙ (i.e., n = 1-6) | SEQ ID NO: 293 |
| Flexible | (Gly)₈ | SEQ ID NO: 298 |
| Flexible | (Gly)₆ | SEQ ID NO: 299 |
| Flexible | KESGSVSSEQLAQFRSLD | SEQ ID NO: 300 |
| Flexible | EGKSSGSGSESKST | SEQ ID NO: 301 |
| Flexible | GSAGSAAGSGEF | SEQ ID NO: 302 |
| Rigid | (EAAAK)ₙ (i.e., n = 1-6) | SEQ ID NO: 303 |
| Rigid | A(EAAAK)₄ALEA(EAAAK)₄A | SEQ ID NO: 304 |
| Rigid | PAPAP | SEQ ID NO: 305 |
| Rigid | AEAAAKEAAAKA | SEQ ID NO: 306 |
| Rigid | (Ala-Pro)ₙ (10 to 34 aa) | SEQ ID NO: 307 |

Other linkers that may be used in the subject fusion proteins include, but are not limited to, SerGly, GGSG (SEQ ID NO: 313), GSGS (SEQ ID NO: 314), GGGS (SEQ ID NO: 315), S(GGS)n (SEQ ID NO: 15) where n is 1-7, GRA, poly(Gly), poly(Ala), GGGSGGG (SEQ ID NO: 316), ESGGGGVT (SEQ ID NO: 317), LESGGGGVT (SEQ ID NO: 318), GRAQVT (SEQ ID NO: 319), WRAQVT (SEQ ID NO: 320), and ARGRAQVT (SEQ ID NO: 321). The hinge regions of the Fc fusions described below may also be considered linkers.

Any conjugation method may be used, or readily adapted, for joining a molecule to an AFFIMER® polypeptide of the present disclosure, including, for example, the methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407.

### Therapeutics

In some embodiments, a fusion protein may comprise a therapeutic molecule (e.g., therapeutic protein) and may be used, for example, to prevent and/or treat a disease in a subject, such as a human subject or other animal subject.

In some embodiments, the fusion protein is for the treatment of an autoimmune disease (a condition in which a subject's immune system mistaken attacks his/her body). Non-limiting examples of autoimmune diseases include myasthenia gravis, pemphigus vulgaris, neuromyelitis optica, Guillain-Barre syndrome, rheumatoid arthritis, systemic lupus erythematosus (lupus), idiopathic thrombocytopenic purpura, thrombotic thrombocytopenic purpura, antiphospholipid syndrome (APS), autoimmune urticarial, chronic inflammatory demyelinating polyneuropathy (CIDP), psoriasis, Goodpasture's syndrome, Graves' disease, inflammatory bowel disease, Crohn's disease, Sjorgren's syndrome, hemolytic anemia, neutropenia, paraneoplastic cerebellar degeneration, paraproteinemic polyneuropathies, primary biliary cirrhosis, stiff person syndrome, vitiligo, warm idiopathic haemolytic anaemia, multiple sclerosis, type 1 diabetes mellitus, Hashimoto's thyroiditis, Myasthenia gravis, autoimmune vasculitis, pernicus anemia, and celiac disease. Other autoimmune diseases are contemplated herein.

In some embodiments, the fusion protein is for the treatment of a cancer. Non-limiting examples of cancers include skin cancer (e.g., melanoma or non-melanoma, such as basal cell or squamous cell), lung cancer, prostate cancer, breast cancer, colorectal cancer, kidney (renal) cancer, bladder cancer, non-Hodgkin's lymphoma, thyroid cancer, endometrial cancer, exocrine cancer, and pancreatic cancer. Other cancers are contemplated herein.

The term treat, as known in the art, refers to the process of alleviating at least one symptom associated with a disease. A symptom may be a physical, mental, or pathological manifestation of a disease. Symptoms associated with various diseases are known. To treat or prevent a particular condition, a conjugate as provided herein (e.g., a fusion protein comprising an AFFIMER® polypeptide linked to a therapeutic molecule) should be administered in an effective amount, which can be any amount used to treat or prevent the condition. Thus, in some embodiments, an effective amount is an amount used to alleviate a symptom associated with the particular disease being treated. Methods are known for determining effective amounts of various therapeutic molecules, for example.

A subject may be any animal (e.g., a mammal), including, but not limited to, humans, non-human primates, canines, felines, and rodents. A “patient” refers to a human subject.

In some embodiments, an AFFIMER® polypeptide is considered “pharmaceutically acceptable,” and in some embodiments, is formulated with a pharmaceutically-acceptable excipient. A molecule or other substance/agent is considered “pharmaceutically acceptable” if it is approved or approvable by a regulatory agency of the Federal government or a state government or listed in the U.S. Pharmacopeia or other generally recognized pharmacopeia for use in animals, including humans. An excipient may be any inert (inactive), non-toxic agent, administered in combination with an AFFIMER® polypeptide. Non-limiting examples of excipients include buffers (e.g., sterile saline), salts, carriers, preservatives, fillers, coloring agents.

### Methods of Use

The fusion proteins of the disclosure are useful in a variety of applications including, but not limited to, therapeutic treatment methods, such as immunotherapy for cancer. In some embodiments, fusion proteins described herein are useful for activating, promoting, increasing, and/or enhancing an immune response, inhibiting tumor growth, reducing tumor volume, inducing tumor regression, increasing tumor cell apoptosis, and/or reducing the tumorigenicity of a tumor. In some embodiments, the polypeptides or agents of the disclosure are also useful for immunotherapy against pathogens, such as viruses. In some embodiments, the fusion proteins described herein are useful for inhibiting viral infection, reducing viral infection, increasing virally-infected cell apoptosis, and/or increasing killing of virus-infected cells. The methods of use may be in vitro, ex vivo, or in vivo methods.

The present disclosure provides methods for activating an immune response in a subject using a fusion protein. In some embodiments, the disclosure provides methods for promoting an immune response in a subject using a fusion protein described herein. In some embodiments, the disclosure provides methods for increasing an immune response in a subject using a fusion protein. In some embodiments, the disclosure provides methods for enhancing an immune response in a subject using a fusion protein. In some embodiments, the activating, promoting, increasing, and/or enhancing of an immune response comprises increasing cell-mediated immunity. In some embodiments, the activating, promoting, increasing, and/or enhancing of an immune response comprises increasing Th1-type responses. In some embodiments, the activating, promoting, increasing, and/or enhancing of an immune response comprises increasing T-cell activity. In some embodiments, the activating, promoting, increasing, and/or enhancing of an immune response comprises increasing CD4+ T-cell activity. In some embodiments, the activating, promoting, increasing, and/or enhancing of an immune response comprises increasing CD8+ T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CU activity and increasing NK cell activity. In some embodiments, the activating, promoting, increasing, and/or enhancing of an immune response comprises inhibiting or decreasing the suppressive activity of Treg cells. In some embodiments, the activating, promoting, increasing, and/or enhancing of an immune response comprises inhibiting or decreasing the suppressive activity of MDSCs. In some embodiments, the activating, promoting, increasing, and/or enhancing of an immune response comprises increasing the number of the percentage of memory T-cells. In some embodiments, the activating, promoting, increasing, and/or enhancing of an immune response comprises increasing long-term immune memory function. In some embodiments, the activating, promoting, increasing, and/or enhancing of an immune response comprises increasing long-term memory. In some embodiments, the activating, promoting, increasing, and/or enhancing of an immune response comprises no evidence of substantial side effects and/or immune-based toxicities. In some embodiments, the activating, promoting, increasing, and/or enhancing of an immune response comprises no evidence of cytokine release syndrome (CRS) or a cytokine storm. In some embodiments, the immune response is a result of antigenic stimulation. In some embodiments, the antigenic stimulation is a tumor cell. In some embodiments, the antigenic stimulation is cancer. In some embodiments, the antigenic stimulation is a pathogen. In some embodiments, the antigenic stimulation is a virally-infected cell.

In vivo and in vitro assays for determining whether a fusion protein activates, or inhibits an immune response are known in the art.

In some embodiments, a method of increasing an immune response in a subject comprises administering to the subject a therapeutically effective amount of a fusion protein described herein, wherein the fusion protein binds human PD-L1. In some embodiments, a method of increasing an immune response in a subject comprises administering to the subject a therapeutically effective amount of a fusion protein described herein, wherein the fusion protein including an AFFIMER® polypeptide that specifically binds to PD-L1. In some embodiments, a method of increasing an immune response in a subject comprises administering to the subject a therapeutically effective amount of an polynucleotide encoding a fusion protein, wherein the polynucleotide encoding a fusion protein, when expressed in the patient, produces a recombinant fusion protein including an PD-L1 binding AFFIMER® polypeptide.

In some embodiments of the methods described herein, a method of activating or enhancing a persistent or long-term immune response to a tumor comprises administering to a subject a therapeutically effective amount of a fusion protein which binds human PD-L1. In some embodiments, a method of activating or enhancing a persistent immune response to a tumor comprises administering to a subject a therapeutically effective amount of a fusion protein described. In some embodiments, a method of activating or enhancing a persistent immune response to a tumor comprises administering to a subject a therapeutically effective amount of a polynucleotide encoding a fusion protein, wherein the polynucleotide encoding a fusion protein, when expressed in the patient, produces a recombinant fusion protein including an PD-L1 binding AFFIMER® polypeptide.

In some embodiments of the methods described herein, a method of inducing a persistent or long-term immunity which inhibits tumor relapse or tumor regrowth comprises administering to a subject a therapeutically effective amount of a fusion protein which binds human PD-L1. In some embodiments, a method of inducing a persistent immunity which inhibits tumor relapse or tumor regrowth comprises administering to a subject a therapeutically effective amount of a fusion protein described herein. In some embodiments, a method of inducing a persistent immunity which inhibits tumor relapse or tumor regrowth comprises administering to a subject a therapeutically effective amount of a polynucleotide encoding a fusion protein, wherein the polynucleotide encoding a fusion protein, when expressed in the patient, produces a recombinant fusion protein including an PD-L1 binding AFFIMER® polypeptide.

In some embodiments of the methods described herein, a method of inhibiting tumor relapse or tumor regrowth comprises administering to a subject a therapeutically effective amount of a fusion protein which binds human PD-L1. In some embodiments, a method of inhibiting tumor relapse or tumor regrowth comprises administering to a subject a therapeutically effective amount of a fusion protein described herein. In some embodiments, a method of inhibiting tumor relapse or tumor regrowth comprises administering to a subject a therapeutically effective amount of a polynucleotide encoding a fusion protein, wherein the polynucleotide encoding a fusion protein, when expressed in the patient, produces a recombinant fusion protein including an PD-L1 binding AFFIMER® polypeptide.

In some embodiments, the tumor expresses or overexpresses a tumor antigen that is targeted by an additional binding entity provided in the fusion protein along with the PD-L1 binding AFFIMER® polypeptide.

In some embodiments, the method of inhibiting growth of a tumor comprises administering to a subject a therapeutically effective amount of a fusion protein described herein. In some embodiments, the subject is a human. In some embodiments, the subject has a tumor, or the subject had a tumor which was removed.

In some embodiments, the tumor is a solid tumor. In some embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In some embodiments, the tumor is a colorectal tumor. In some embodiments, the tumor is an ovarian tumor. In some embodiments, the tumor is a lung tumor. In some embodiments, the tumor is a pancreatic tumor. In some embodiments, the tumor is a melanoma tumor. In some embodiments, the tumor is a bladder tumor.

To further illustrate, the subject fusion proteins can be used to treat patients suffering from cancer, such as osteosarcoma, rhabdomyosarcoma, neuroblastoma, kidney cancer, leukemia, renal transitional cell cancer, bladder cancer, Wilm's cancer, ovarian cancer, pancreatic cancer, breast cancer (including triple negative breast cancer), prostate cancer, bone cancer, lung cancer (e.g., small cell or non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer, breast cancer or gastric cancer. In some embodiments of the disclosure, the cancer is metastatic cancer, e.g., of the varieties described above.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The present disclosure also provides pharmaceutical compositions comprising a fusion protein described herein and a pharmaceutically acceptable vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified fusion protein of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, a fusion protein described herein is lyophilized and/or stored in a lyophilized form. In some embodiments, a formulation comprising a fusion protein described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22nd Edition, 2012, Pharmaceutical Press, London).

The pharmaceutical compositions of the present disclosure can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The fusion proteins described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In some embodiments, pharmaceutical formulations include a fusion protein of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, sustained-release preparations comprising fusion proteins described herein can be produced. Suitable examples of sustained-release preparations include semi-permeable matrices of solid hydrophobic polymers containing a fusion protein, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

For the treatment of a disease, the appropriate dosage of a fusion protein of the present disclosure depends on the type of disease to be treated, the severity and course of the disease, the responsiveness of the disease, whether the fusion protein is administered for therapeutic or preventative purposes, previous therapy, the patient's clinical history, and so on, all at the discretion of the treating physician. The fusion protein can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In some embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In some embodiments, the dosage of the fusion protein is from about 0.1 mg to about 20 mg/kg of body weight. In some embodiments, the dosage of the fusion protein is about 0.1 mg/kg of body weight. In some embodiments, the dosage of the fusion protein is about 0.25 mg/kg of body weight. In some embodiments, the dosage of the fusion protein is about 0.5 mg/kg of body weight. In some embodiments, the dosage of the fusion protein is about 1 mg/kg of body weight. In some embodiments, the dosage of the fusion protein is about 1.5 mg/kg of body weight. In some embodiments, the dosage of the fusion protein is about 2 mg/kg of body weight. In some embodiments, the dosage of the fusion protein is about 2.5 mg/kg of body weight. In some embodiments, the dosage of the fusion protein is about 5 mg/kg of body weight. In some embodiments, the dosage of the fusion protein is about 7.5 mg/kg of body weight. In some embodiments, the dosage of the fusion protein is about 10 mg/kg of body weight. In some embodiments, the dosage of the fusion protein is about 12.5 mg/kg of body weight. In some embodiments, the dosage of the fusion protein is about 15 mg/kg of body weight. In some embodiments, the dosage can be given once or more daily, weekly, monthly, or yearly. In some embodiments, the fusion protein is given once every week, once every two weeks, once every three weeks, or once every four weeks.

In some embodiments, a fusion protein may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the fusion protein is administered for 3, 4, 5, 6, 7, 8, or more cycles. For example, the fusion protein is administered every 2 weeks for 6 cycles, the fusion protein is administered every 3 weeks for 6 cycles, the fusion protein is administered every 2 weeks for 4 cycles, the fusion protein is administered every 3 weeks for 4 cycles, etc. Dosing schedules can be decided upon and subsequently modified by those skilled in the art.

Thus, the present disclosure provides methods of administering to a subject the polypeptides or agents described herein comprising using an intermittent dosing strategy for administering one or more agents, which may reduce side effects and/or toxicities associated with administration of a fusion protein, chemotherapeutic agent, etc. In some embodiments, a method for treating cancer in a human subject comprises administering to the subject a therapeutically effective dose of a fusion protein in combination with a therapeutically effective dose of a chemotherapeutic agent, wherein one or both of the agents are administered according to an intermittent dosing strategy. In some embodiments, the intermittent dosing strategy comprises administering an initial dose of a fusion protein to the subject and administering subsequent doses of the fusion protein about once every 2 weeks. In some embodiments, the intermittent dosing strategy comprises administering an initial dose of a fusion protein to the subject, and administering subsequent doses of the fusion protein about once every 3 weeks. In some embodiments, the intermittent dosing strategy comprises administering an initial dose of a fusion protein to the subject, and administering subsequent doses of the fusion protein about once every 4 weeks. In some embodiments, the fusion protein is administered using an intermittent dosing strategy and the chemotherapeutic agent is administered weekly.

In some embodiments, the disclosure also provides methods for treating subjects using a fusion protein of the disclosure, wherein the subject suffers from a viral infection. In some embodiments, the viral infection is infection with a virus selected from the group consisting of human immunodeficiency virus (HIV), hepatitis virus (A, B, or C), herpes virus (e.g., VZV, HSV-I, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus or arboviral encephalitis virus.

In some embodiments, the disclosure provides methods for treating subjects using a fusion protein thereof of the disclosure, wherein the subject suffers from a bacterial infection. In some embodiments, the bacterial infection is infection with a bacterium selected from the group consisting of Chlamydia, rickettsial bacteria, mycobacteria, staphylococci, streptococci, pneumonococci, meningococci and gonococci, Klebsiella, Proteus, Serratia, Pseudomonas, Legionella, Corynebacterium diphtheriae, Salmonella, bacilli, Vibrio cholerae, Clostridium tetan, Clostridium botulinum, Bacillus anthricis, Yersinia pestis, Mycobacterium leprae, Mycobacterium lepromatosis, and Borrelia.

In some embodiments, the disclosure provides methods for treating subjects using a fusion protein of the disclosure, wherein the subject suffers from a fungal infection. In some embodiments, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In some embodiments, the disclosure provides methods for treating subjects using a fusion protein of the disclosure, wherein the subject suffers from a parasitic infection. In some embodiments, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii and Nippostrongylus brasiliensis.

### Polynucleotides

A polynucleotide (also referred to as a nucleic acid) is a polymer of nucleotides of any length, and may include deoxyribonucleotides, ribonucleotides, modified nucleotides or bases, and/or their analogs, or any substrate that can be incorporated into a polymer by DNA or RNA polymerase. In some embodiments, a polynucleotide herein encodes a polypeptide, such as a fusion protein comprising a HSA binding AFFIMER® polypeptide and a PD-L1 binding AFFIMER® polypeptide. As known in the art, the order of deoxyribonucleotides in a polynucleotide determines the order of amino acids along the encoded polypeptide (e.g., protein).

A polynucleotide sequence may be any sequence of deoxyribonucleotides and/or ribonucleotides, may be single-stranded, double-stranded, or partially double-stranded. The length of a polynucleotide may vary and is not limited. Thus, a polynucleotide may comprise, for example, 2 to 1,000,000 nucleotides. In some embodiments, a polynucleotide has a length of 100 to 100,000, a length of 100 to 10,000, a length of 100 to 1,000, a length of 100 to 500, a length of 200 to 100,000, a length of 200 to 10,000, a length of 200 to 1,000, or a length of 200 to 500 nucleotides.

A vector herein refers to a vehicle for delivering a molecule to a cell. In some embodiments, a vector is an expression vector comprising a promoter (e.g., inducible or constitutive) operably linked to a polynucleotide sequence encoding a polypeptide. Non-limiting examples of vectors include viral vectors (e.g., adenoviral vectors, adeno-associated virus vectors, and retroviral vectors), naked DNA or RNA expression vectors, plasmids, cosmids, phage vectors, DNA and/or RNA expression vectors associated with cationic condensing agents, and DNA and/or RNA expression vectors encapsulated in liposomes. Vectors may be transfected into a cell, for example, using any transfection method, including, for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics technology (biolistics).

### EXAMPLES

### Example 1. Human Serum Albumin (HSA) and Mouse Serum Albumin (MSA) AFFIMER® Binder Selection

Selection of HSA or MSA binding phage from the AFFIMER® polypeptide library was carried out using approximately 1×10¹² phage added from a library of size approximately 6×10¹⁰ diversity.

The HSA binding peptides of the disclosure were identified by selection from the phage display library comprising random loop sequences nine amino acids in length displayed in a constant AFFIMER® framework backbone based upon the sequence for SQT. Suspensions of phage were incubated with target antigen (either biotinylated antigen captured on streptavidin beads or unbiotinylated antigen captured on a plate). Unbound phage were then washed away and, subsequently, bound phage were eluted by incubating the antigen with low pH, followed by high pH. Then, E. coli were infected with released, pH neutralized phage and a preparation of first round phage was obtained. The cycle was repeated two or three times. In order to enrich for targeting phage, the stringency conditions were increased in the later rounds of selection. Increased stringency conditions included increasing the number of wash steps, reducing the antigen concentration, and/or preselecting with blocked streptavidin beads or wells coated with blocking reagent.

Antigens used herein for phage selections were HSA (Sigma; A3782), and MSA (Alpha Diagnostics; ALB13-N-25). Antigen biotinylation was carried out in-house using the EZ Link Sulfo-NHS-LC Biotin kit (Pierce).

Following selection by successive rounds of phage amplification, HSA and MSA binding clones were identified by a phage ELISA as described below. Following phage selections, individual bacterial clones containing the phagemid vector were moved from titration plates into 96 well cell culture format. Recombinant phage particles that displayed HSA AFFIMER® polypeptide fused to the gene-III minor coat protein were released into the culture supernatant following helper phage rescue and overnight growth. The phage contained in the supernatants were subsequently screened for binding to antigen by ELISA. Phage-displaying AFFIMER® protein binding to antigen immobilized on a plate was detected with an HRP-conjugated anti-M13 monoclonal antibody (GE Healthcare), and the ELISA was developed using 1-step Ultra TMB-ELISA substrate (Thermo Scientific).

### Example 2. AFFIMER® E. coli Protein Production

All AFFIMER® polypeptides expressed in E. coli have been cloned with a C-terminal hexa-HIS tag (HHHHHH; SEQ ID NO: 292) to simplify protein purification with immobilized metal affinity chromatography resin (IMAC resin). When required, additional peptide sequences can be added between the AFFIMER® protein and the HIS tag such as MYC (EQKLISEEDL SEQ ID NO: 295) for detection or a TEV protease cleavage site (ENLYFQ(G/S) SEQ ID NO: 296) to allow for the removal of tags. AFFIMER® proteins were expressed from E. coli and purified using IMAC, IEX, and SEC. AFFIMER® monomer purification from E. coli was performed by transforming the expression plasmid pD861 (Atum) into BL21 E. coli cells (Millipore) using the manufacturer's protocol. The total transformed cell mixture was plated onto LB agar plates containing 50 μg/ml kanamycin (AppliChem) and incubated at 37° C. overnight. The following day, the lawn of transformed E. coli was transferred to a sterile flask of 1× terrific broth media (Melford) and 50 μg/ml kanamycin and incubated at 30° C. shaking at 250 rpm. Expression was induced with 10 mM rhamnose (Alfa Aesar) once the cells reached an optical density OD₆₀₀ of approximate 0.8-1.0. The culture was then incubated for a further 5 hours at 37° C. Cells were harvested by centrifuging and lysing the resulting cell pellet. AFFIMER® protein purification was performed using batch bind affinity purification of His-tagged protein. Specifically, nickel agarose affinity resin (Super-NiNTA500; Generon) was used. The resin was washed with NPI20 buffer (50 mM sodium phosphate, 0.5M NaCl, 20 mM imidazole) and the bound protein was eluted with 5 column volumes (CV) of NPI400 buffer. Eluted protein was then purified by cation exchange using an CM FF ion exchange column (GE) in running buffer 20 mM sodium acetate pH 5.2 for clone HSA-31 and 25 mM MES pH 6.0 for clone HSA-41. Both protein purifications further included a 0.1% triton 114× (Sigma) wash step and the protein was eluted with a 1M NaCl linear gradient. A third stage purification was performed on a preparative SEC performed using the HiLoad 26/600 Superdex 75 pg (GE Healthcare) run in PBS 1× buffer. Expression and purity of clone HSA-41 and HSA-31 was analyzed using SEC-HPLC with an Acclaim SEC-300 column (Thermo) using a PBS 1× mobile phase. The protein yield was estimated using Nanodrop (Thermo) A280 readings and the final product was run on an SDS-PAGE Bolt Bis Tris plus 4-12% gel (Thermo) in Novex™ 20× Bolt™ MES SDS running buffer (Thermo) at 200 volts, with samples heated in reducing buffer. Protein bands on the gel were stained with Quick Commassie (Generon). PageRuler prestained protein molecular weight marker (Thermo) was run on the gel to estimate the molecular weight of the fusion proteins following the three-stage purification.

### Example 3. Serum Albumin Binding AFFIMER® Protein Characterization

AFFIMER® protein affinity to serum for human, mouse, and cynomolgus was assessed by Biolayer Interferometry (Octet) at both pH 6.0 and pH 7.4. Biotinylated antigen was captured onto SA sensors at 1 μg/ml for 600 seconds in a buffer comprising PBS-T (0.01% Tween 20)+1% casein at either pH 6.0 or 7.4. Association was carried out for 300 seconds and dissociation for 600 seconds, and regeneration was performed using 10 mM glycine pH 1.5 (GE Healthcare) for 3×5 seconds. All steps were carried out at 1000 rpm and 25° C. Purified AFFIMER® protein in two-fold serial dilutions was analyzed at a starting concentration of approximately 10×K_{D} value. Kinetics analyses were carried out using the Octet data analysis software, subtracting the reference sensor (loaded with antigen), aligning the Y-axis to baseline, and using inter-step correction to align association to dissociation. Savitzky-Golay filtering was applied and the data processed. Analysis of the data was carried out with a 1:1 model, global fit, Rₘₐₓ unlinked by sensor.

### Example 4. PD-L1 Binding AFFIMER® Protein Half-Life Extended In-Line Fusion (ILF) Dimers

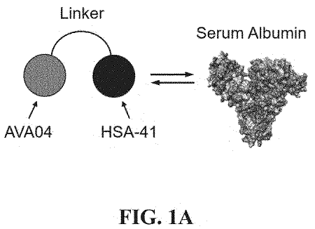

The half-life of three (3) PD-L1 binding AFFIMER® proteins AVA04-236 (SEQ ID NO: 277 and 278), AVA04-261 (SEQ ID NO: 279 and 280), and AVA04-269 (SEQ ID NO: 281 and 282), was extended by genetically fusing to AVA-41 at the N- or C-terminus. A schematic representation of half-life extended PD-L1 AFFIMER® proteins formatted as dimer genetic fusions using a rigid (A(EAAAK)₆ (SEQ ID NO: 294)) or flexible (G4S)₆ (SEQ ID NO: 293) repetitive genetic linkers is provided in FIG. 1A. A table of ILF orientation and nomenclature supplied in Table 8 below. Table 7 shows examples of half-life extension in-line fusions with PD-L1 binding AFFIMER® proteins.

ILF dimer production from E. coli was purified using three stages: affinity capture, IEX, and preparative SEC. Final ILF protein purity was assessed using SEC-HPLC and shown to be >95% pure (FIG. 1B). Biacore kinetic analysis showed both AFFIMER® proteins genetically fused were able to engage target antigens, human PD-L1-Fc (R&D Systems), and HSA (Sigma) (Table 9). To analyze PD-L1-Fc binding kinetics, a Biacore T200 kinetic analysis was performed using running buffer HBS-EP+ and series S sensor CM5 (GE Healthcare) chip Fc2 immobilized with PD-L1-Fc (R&D Systems) in 10 mM sodium acetate pH 4.0 using amine coupling reagents (GE Healthcare). The concentration titration of ILF AFFIMER® proteins was run as analyte at a flow rate of 30 μl/min. Regenerated PD-L1-Fc was immobilized on a surface with 3 mM NaOH (GE healthcare) for 20 seconds at 20 μl/min flow rate. The Fc2-1 data blank was subtracted and fit to a 1:1 Langmuir binding model (Biacore evaluation software; GE) to calculate an apparent K_{D} value.

TABLE 8
Nomenclature of PD-L1 (AVA04) binding AFFIMER ® ILF
proteins half-life extended with HSA binding
AFFIMER ® proteins

| | | | Linker | |
|---|---|---|---|---|
| AFFIMER ® | AVA04 | | SEQ ID | AFFIMER ® |
| Format | AFFIMER ® | Linker | NO: | XT |
| AVA04-236 | AVA04-236 | (G4S)₆ | 293 | AVA-41 |
| XT7 | | | | |
| AVA04-236 | AVA04-236 | SEQ ID)₆ | 294 | AVA-41 |
| XT8 | | | | |
| AVA04-261 | AVA04-261 | (G4S)₆ | 293 | AVA-41 |
| XT9 | | | | |
| AVA04-261 | AVA04-261 | A(EAAAK)₆ | 294 | AVA-41 |
| XT10 | | | | |
| AVA04-269 | AVA04-269 | (G4S)₆ | 293 | AVA-41 |
| XT11 | | | | |
| AVA04-269 | AVA04-269 | A(EAAAK)₆ | 294 | AVA-41 |
| XT12 | | | | |

TABLE 9
BIACORE ™ Kinetic Analysis of AFFIMER ® Proteins

| | | HuSA kinetics | | | PD-L1 Kinetics | | |
|---|---|---|---|---|---|---|---|
| | Dimer | ka | kd | KD | ka | kd | KD |
| Protein | linker | (1/Ms) | (1/s) | (nM) | (1/Ms) | (1/s) | (nM) |
| HSA-41 | | 1.48E+06 | 3.54E−03 | 2.4 | | | |
| AVA04-236 | | | | | 1.80E+06 | 9.77E−03 | 5.43 |
| AVA04-236 XT7 | Flexible | 2.53E+05 | 3.36E−03 | 13.3 | 1.57E+06 | 7.63E−03 | 4.86 |
| AVA04-236 XT8 | rigid | 2.67E+05 | 2.52E−03 | 9.5 | 1.52E+06 | 6.95E−03 | 4.57 |
| AVA04-261 | | | | | 3.68E+06 | 1.09E−02 | 2.95 |
| AVA04-261 XT9 | Flexible | 2.69E+05 | 3.57E−03 | 13.3 | 3.95E+05 | 7.07E−03 | 17.9 |
| AVA04-261 XT10 | rigid | 2.99E+05 | 2.91E−03 | 9.7 | 8.75E+05 | 8.26E−03 | 9.44 |
| AVA04-269 | | | | | 2.05E+06 | 3.45E−03 | 1.68 |
| AVA04-269 XT11 | Flexible | 1.51E+05 | 2.34E−03 | 15.5 | 7.96E+05 | 3.54E−03 | 4.45 |
| AVA04-269 XT12 | rigid | 1.77E+05 | 2.28E−03 | 12.9 | 1.14E+06 | 4.31E−03 | 3.8 |

### Example 5. PD-L1 Binding AFFIMER® Dimers Half-Life Extended in Line Fusions Trimers

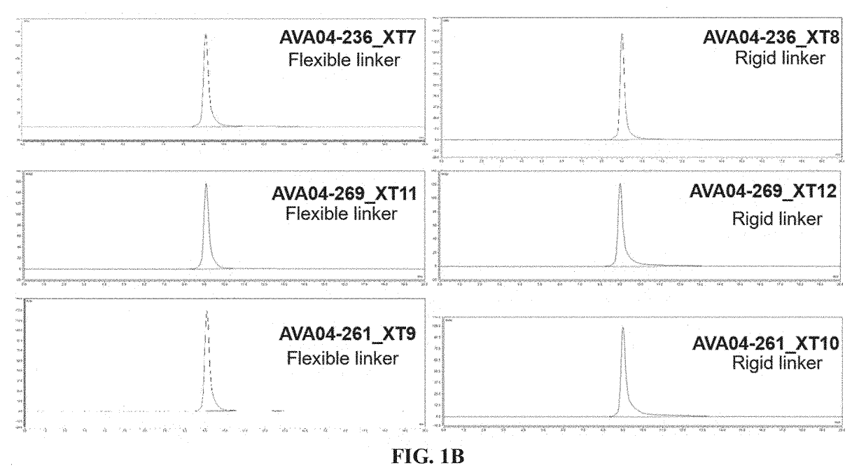

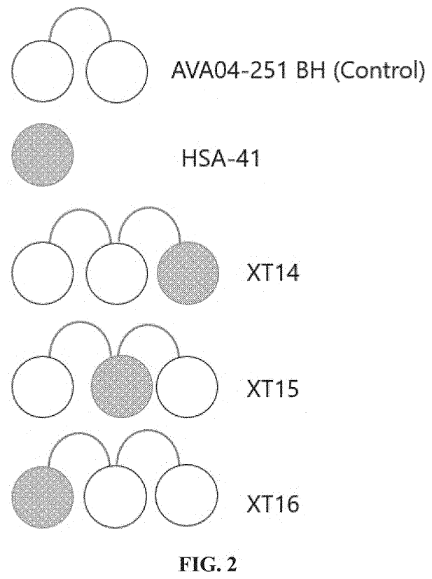

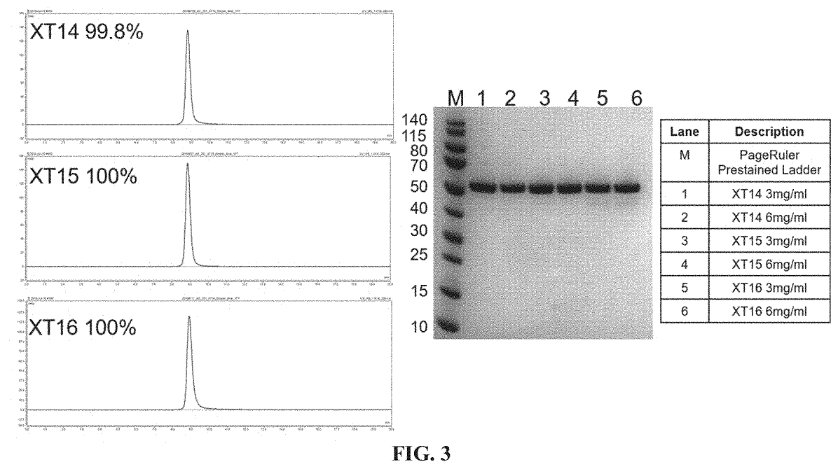

FIG. 2 shows a schematic representation of PD-L1 binding AFFIMER® dimer genetically fused with rigid linkers A(EAAAK)₆ (SEQ ID NO: 294) to HSA-41. ILF production in E. coli was performed using protein purified using affinity capture, IEX, and preparative SEC. Protein purity was assessed using SDS-PAGE and SEC-HPLC. The AFFIMER® proteins were found to be 99.8% to 100% pure (FIG. 3). Biacore kinetic analysis showed that genetically fused AFFIMER® dimers are able to engage both their target proteins (FIG. 4A). The AVA04 AFFIMER® protein was found to bind PD-L1 and HSA-41 and engage HSA. Biacore analyses were carried out to analyze HSA binding and as described in Example 8 to analyze PD-L1-Fc binding (Table 10).

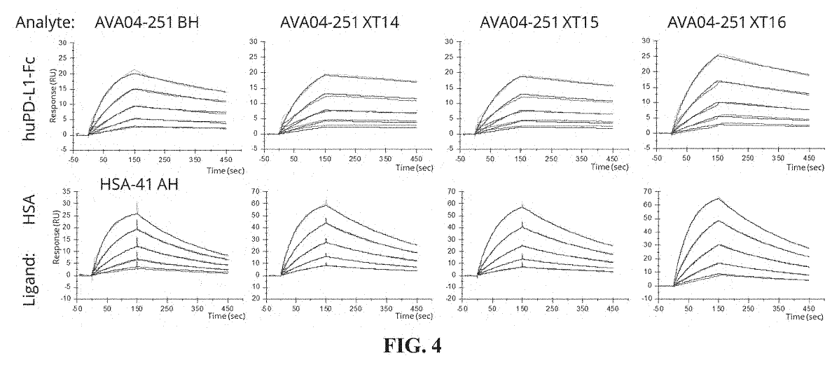

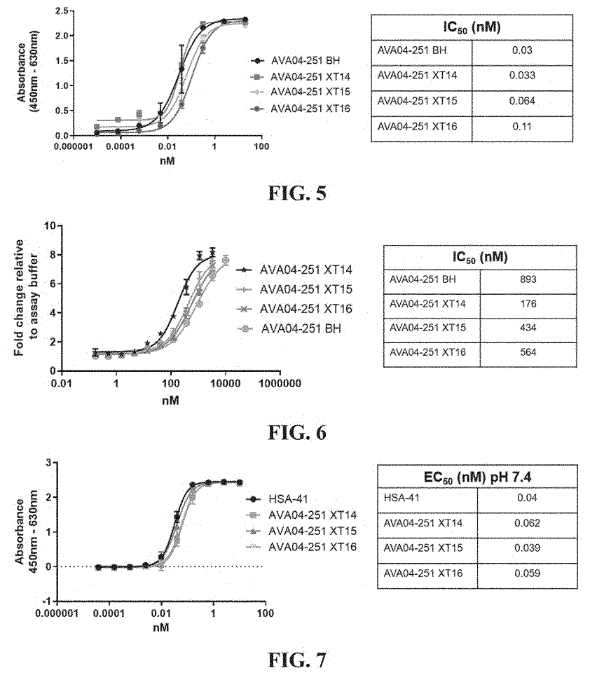

To evaluate if the addition of HSA-41 at various positions in an AFFIMER® in-line fusion format impacted binding of AVA04-251 to human PD-L1, a PD-L1 binding ELISA was performed with the three (3) ILF formatted AFFIMER® proteins (FIG. 5). Briefly, human PD-L1-Fc (R&D Systems) chimeric protein was coated on 96 well plates at 0.5 mg/ml in carbonate buffer. After saturation with 5% casein/PBS buffer, the plates were washed and a dilution of AFFIMER® proteins or controls were incubated for 90 minutes. Plates were then washed and a biotinylated polyclonal antibody anti-cystatin A (R&D Systems) was added for 1 hour. Plates were washed and AFFIMER® proteins were detected using streptavidin-HRP. After a last washing step, TMB was added for the development of the experiment and plates were read at 450 nm. The three (3) constructs tested exhibit similar EC₅₀ (ranging from 0.03 to 0.1 nM) and are identical to the anti-PD-L1 parental ILF dimer molecule (AVA04-251 BH). This was confirmed when a PD-1/PD-L1 blockade Bioassay (Promega) of the half-life extended AFFIMER® proteins compared to the parental molecule was performed (FIG. 6). The PD-1/PD-L1 blockade Bioassay (Promega) assay was run according to manufacturer instructions in duplicate and showed the three (3) constructs tested have similar EC₅₀ values (within 2-fold difference) and are identical to the parental dimer molecule (AVA04-251 BH).

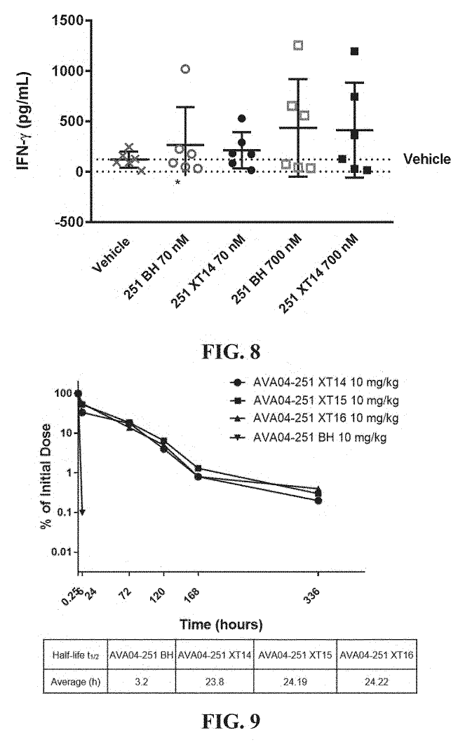

Similarly, the binding to human serum albumin was assessed for the three (3) half-life extended AFFIMER® constructs using an ELISA at pH 7.4. Briefly, HSA was coated in 96 well plates at 1 mg/ml at pH 7.5. After saturation with 5% PBS Casein pH 7.5, plates were washed and a dilution of AFFIMER® proteins or controls were incubated for 90 minutes. Plates were then washed and a biotinylated polyclonal antibody anti-cystatin A (R&D Systems) was added for 1 hour. Plates were washed and AFFIMER® proteins were detected using streptavidin-HRP. After a last washing step, TMB was added for the development of the experiment and the plates were read at 450 nm. The three (3) constructs tested exhibit similar EC₅₀ (ranging from 0.03 to 0.06 nM) and are identical to the parental molecule (HSA-41) (FIG. 7).

TABLE 10
BIACORE ™ Kinetic Analysis of AFFIMER ® Proteins (PD-L1-Fc, HSA)

| | rhPD-L1-Fc | | | | | HSA | | | | |
|---|---|---|---|---|---|---|---|---|---|---|
| | ka | kd | KD | Rmax | Chi² | ka | kd | KD | Rmax | Chi² |
| AFFIMER ® | (1/Ms) | (1/s) | (nM) | (RU) | (RU²) | (1/Ms) | (1/s) | (nM) | (RU) | (RU²) |
| 251_BH | 7.52E+05 | 1.29E−03 | 1.71 | 23 | 0.154 | | | | | |
| parent | | | | | | | | | | |
| AVA04-251 | 8.34E+05 | 4.20E−04 | 0.504 | 24.9 | 0.355 | 3.22E+05 | 2.82E−03 | 8.75 | 73.3 | 0.211 |
| XT14 | | | | | | | | | | |
| AVA04-251 | 8.63E+05 | 6.07E−04 | 0.704 | 24.3 | 0.307 | 5.57E+05 | 2.90E−03 | 5.20 | 75.6 | 0.202 |
| XT15 | | | | | | | | | | |
| AVA04-251 | 4.03E+05 | 9.44E−04 | 2.34 | 34.2 | 0.194 | 8.21E+05 | 3.24E−03 | 3.95 | 78.5 | 0.228 |
| XT16 | | | | | | | | | | |
| HSA-41 | | | | | | 8.48E+05 | 4.35E−03 | 5.13 | 32.1 | 0.255 |
| parent | | | | | | | | | | |

### Example 6. Mixed Lymphocyte Reaction of Half-Life Extended ILF Trimer

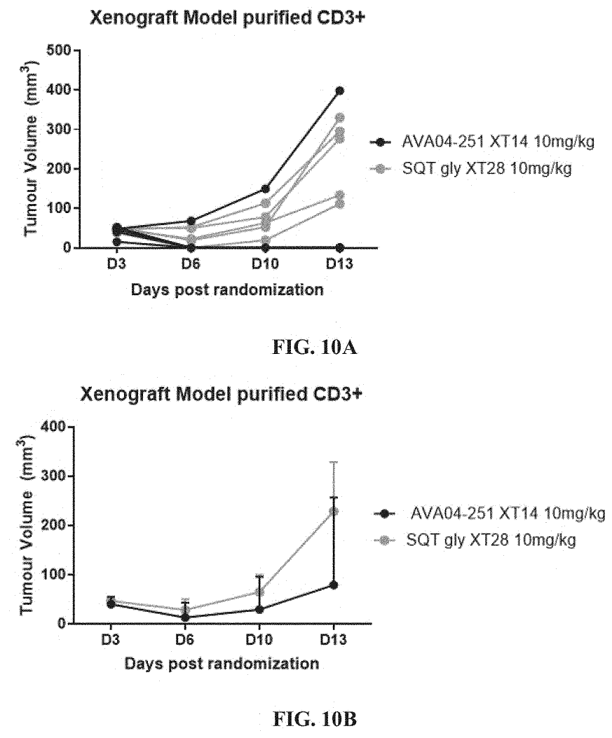

AVA04-251 XT in-line fusion formatted AFFIMER® proteins were tested in a mixed lymphocyte reaction (MLR) assay (FIG. 8). Briefly, dendritic cells (DC) derived from monocytes were prepared from CD14+ monocytes cultured for seven (7) days. Immature DCs were used on day 7 and cultured together with allogeneic T-cells (negative isolation) and reference substance or vehicle control (RPMI-10 media). Cells were cultured for 4 days and IFNγ was measured in supernatants at the end of the culture period by ELISA. Data are presented as mean+/−S.E.M. pg/ml or normalised to vehicle control (n=6). The dotted line represents mean vehicle (RPMI-10) value. The half-life formatted AFFIMER® AVA04-251 XT14 was found to increase the level of IFNγ similarly to its control (non-half-life extended) (FIG. 8).

### Example 7. Pharmacokinetics Profile of PD-L1 Binding Half-Life Extended In-Line Fusion AFFIMER® Proteins in Mice

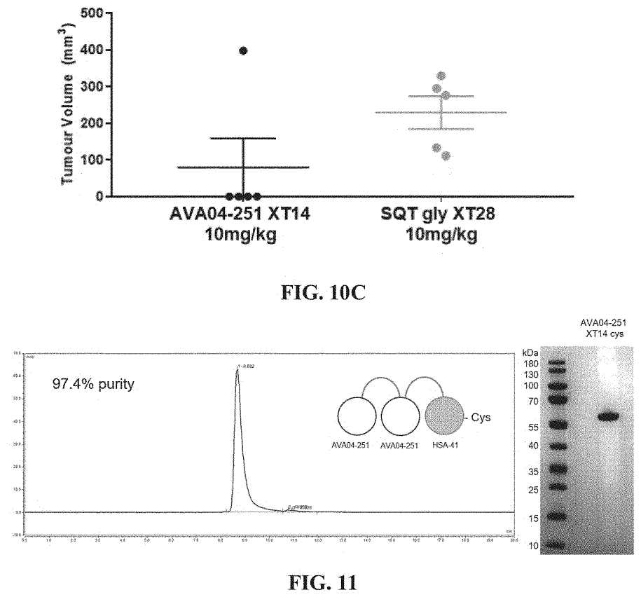

The ILF AVA04-251 trimers with half-life extension were tested in a pharmacokinetic study in C57/B16 mice. As described FIG. 9, mice were injected intravenously (IV) at 10 mg/kg. Six mice were used and serum was collected at nine time points (0, 0.25, 6, 24, 72, 120, 168, and 336 hours). The serum samples for each time point were pooled and analyzed by sandwich ELISA using the purified molecules injected as a reference standard. Results were expressed as the percentage of initial dose at 15 minutes. AFFIMER® ILF without half-life extension (AVA04-251 BH) had a fast clearance (t_{1/2} 3.2 hours) whereas ILF AVA04-251 XT formats all showed half-life extension, estimated in the beta phase (ranging from 23.8-24.2 hours).

### Example 8. Mouse Xenograft Model Testing of AFFIMER® ILF Trimer

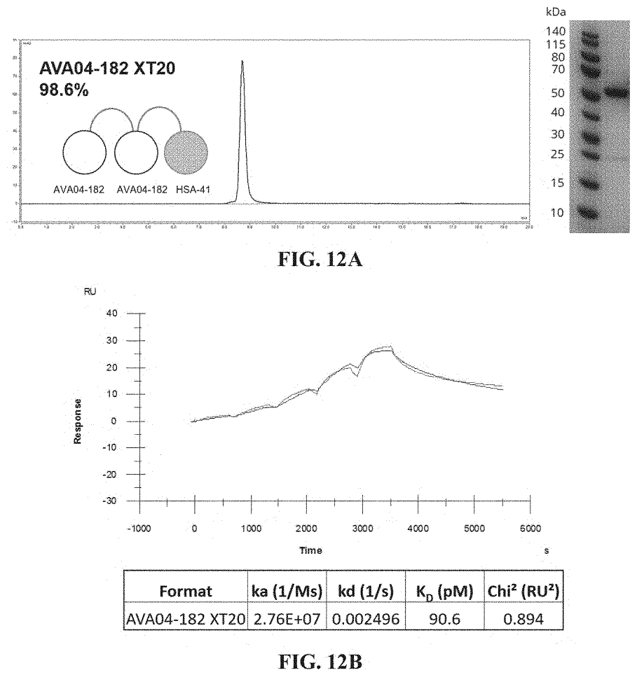

PBMCs were isolated from one healthy donor. Total T-cells were isolated and expanded on A375 cells for two rounds for 7 to 10 days in complete medium supplemented with IL-2. Mice (n=10) were inoculated subcutaneously at the right flank region with A375 tumor cells and activated T-cells (0.2 ml in PBS) for tumor development. The treatments were started one-hour post cell inoculation. AVA04-251 XT14 (SEQ ID NO: 283) purified protein was administered two (2) times a week for three (3) weeks. Overall, tumor growth inhibition was shown for both treatments when compared to controls at day 13 post-randomization. More than 70% of mice treated with AVA04-251 XT14 (SEQ ID NO: 283) had a reduced tumor size compared to the control group, which was given the non-binding AFFIMER® ILF SQT gly XT28 (SEQ ID NO: 288) (FIGS. 10A-10C).

### Example 9. AVA04 Half-Life Extended ILF Trimer C-Terminal Cys AFFIMER® Protein Expression

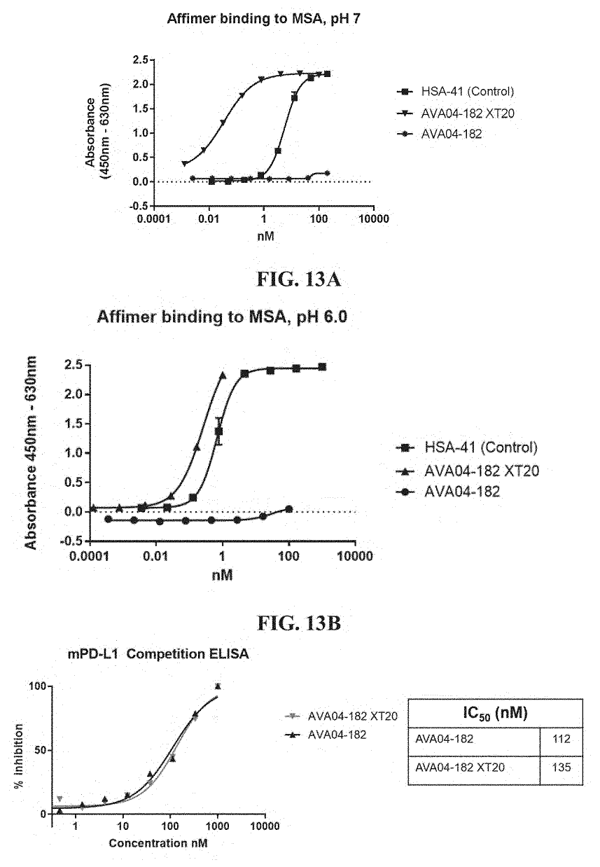

The half-life extended trimer was synthethesized to further comprise a C-terminal cysteine amino acid following the C-terminal 6×His tag by quick change mutagenesis (Agilent) to create AVA04-251 XT14 cys (SEQ ID NO: 126). The AFFIMER® protein was produced from E. coli and purified with affinity, IEX, and preparative size exclusion. Characterization of the purified protein under reducing conditions with 2 mM TCEP showed that the purity of the final protein is >97% (FIG. 11). AFFIMER® ILF proteins can therefore be produced with a free cysteine for subsequent conjugation using maleimide chemistry to enable the generation of AFFIMER® protein-drug conjugates.

### Example 10. Example of an Anti-Mouse PD-L1 Binder Half-Life Extended In-Line Fusion AFFIMER® Trimer

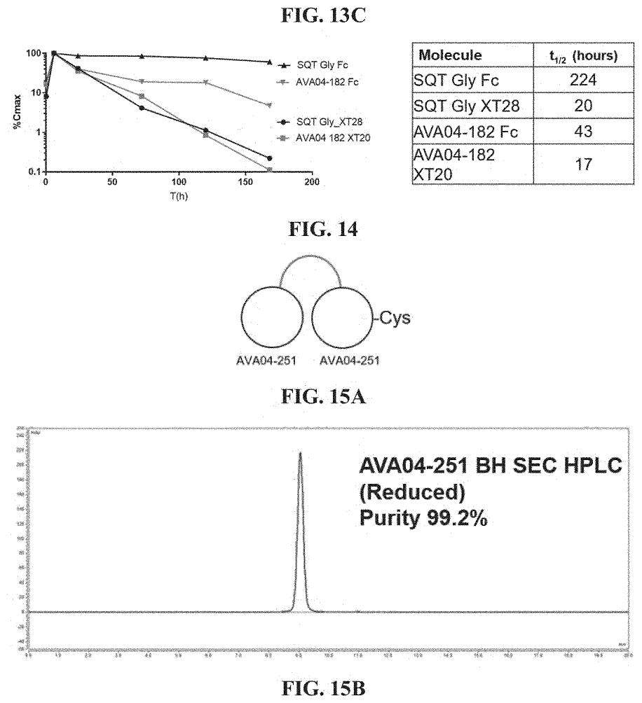

AVA04-182 XT20 half-life extended ILF trimer was produced from E. coli. SDS-PAGE and SEC-HPLC analyses were run as described in Example 2 and showed final protein purity of over >98% (FIG. 12A). Purified protein was run on Biacore to assess its affinity to mouse PD-L1-Fc tagged recombinant antigen (R&D systems). Antigen was captured using a Protein A chip (GE Healthcare) and the AFFIMER® ILF format was run as an analyte using single cycle kinetics titrating from a maximum concentration of 1 nM, and regenerating using 10 mM glycine pH 1.5 (GE Healthcare). Fc2-1 kinetic data was blank subtracted and fit to a 1:1 Langmuir binding model (BIAcore Evaluation software; GE Healthcare) to calculate a K_{D} value of 90.6 pM, confirming that the addition of the half-life extending AFFIMER® protein in this format did not affect the AVA04-182 binding to mouse PD-L1 target antigen (FIG. 12B).

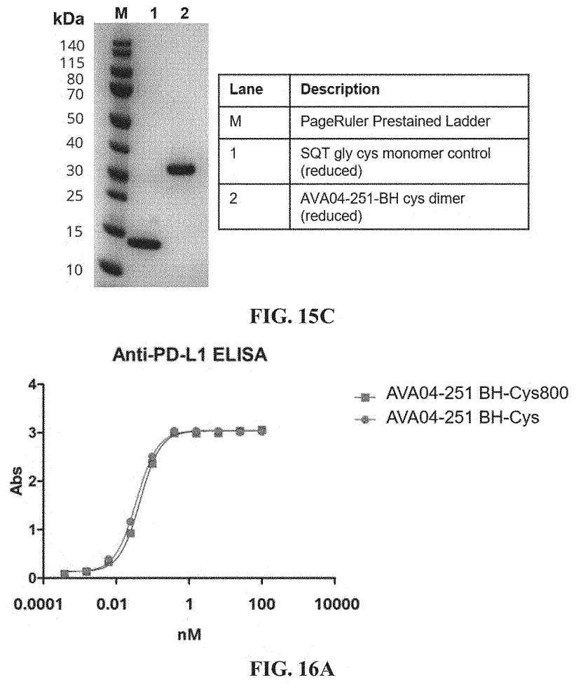

AVA04-182 XT20 ILF was evaluated in an ELISA for its capacity to bind HSA at pH 7.4 and pH 6.0 (as described in the Example 4). FIGS. 13A and 13B show that AVA04-182 XT20 retained the capacity of HSA-41 to bind MSA. In addition, to evaluate if the half-life extended AFFIMER® protein was functional, a competitive ELISA (mPD-1/mPD-L1) was performed. Briefly, PD-1 was coated overnight on the plate at 1 μg/ml in carbonate buffer. Then plates were saturated using 5% Casein/PBS buffer. In the meantime, mPD-L1 was pre-incubated with a dilution of half-life extended AFFIMER® protein and its control. After saturation, the mix was added to the plates and incubated for 90 minutes. Plates were then washed and the detection polyclonal antibody, biotinylated anti-PD-L1, was added. After washing the plates, streptavidin-HRP was added for 30 minutes. After a final wash, development of the reaction was performed using TMB (Pierce) and the plates were read using a plate reader at 450 nm (FIG. 13C). The figure shows that half-life extended AFFIMER® protein has a similar neutralizing capacity to its parental molecule.

As a proof of concept, a pharmacokinetic study was performed in mice. Twelve animals per group were injected intraperitoneally (IP) with 25 mg/kg AFFIMER® protein and three animals were used per timepoint. At eight (8) timepoints, serum was drawn, up to 336h post injection. Pooled serum was analyzed using an ELISA to quantify the level of AFFIMER® protein in serum. The pharmacokinetic profile of the half-life extended AFFIMER® protein showed a half-life of ˜17 hours in this study (FIG. 14).

### Example 11: Biodistribution of AVA04-251 BH-800CW in a A375 Mouse Xenograft Model

Whether anti-PD-L1 AFFIMER® proteins are targeted to tumors expressing human PD-L1 was assessed in a mouse xenograft model examining the biodistribution of IR dye-conjugated AFFIMER® protein over time using fluorescence imaging. AVA04-251 BH cys and AVA04-251 XT14 cys were conjugated to IRDye 800CW (LI-COR) with maleimide chemistry to modify the accessible amino groups on the protein. AFFIMER® proteins were diluted to 1 mg/ml in 50 mM MES pH 6, 150 mM NaCl, 1 mM TCEP and incubated with IRDye 800CW (4 mg/mL in water) at a stoichiometry of 9:1 dye:protein for 2 hours in dark conditions at room temperature (˜23° C.). Free dye was separated from dye-conjugated AFFIMER® proteins using a 5 mL Zeba Spin Desalting Column (MWCO 7000; Pierce) according to the manufacturer's instructions. The dye:protein ratio was calculated based on the absorbance at 280 and 780 nm according to the equation:

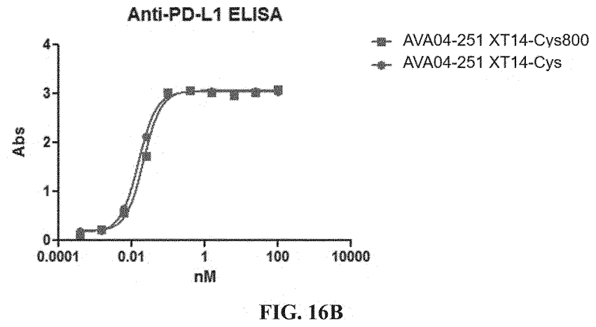

Dye:protein ratio=(A780/εDye)/(A280−(0.03×A780))/εprotein,

where 0.03 is the correction factor for the absorbance of IRDye 800CW at 280 nm, and εDye and ε protein are molar extinction coefficients for the dye 270,000 M⁻¹ cm⁻¹ and protein 39871 M⁻¹ cm⁻¹ for AVA04-251 BH Cys and 37626 M⁻¹ cm⁻¹ for AVA04-251 XT14 cys, respectively. FIGS. 15A-15C show the format schematic and purity of conjugated material using SEC-HPLC and SDS-PAGE analytical methods.

The binding of dye-conjugated AVA04-251 BH-800 or AVA04-251 XT14-800 to recombinant human PD-L1 was compared to non-conjugated AFFIMER® protein using a PD-L1 binding ELISA.

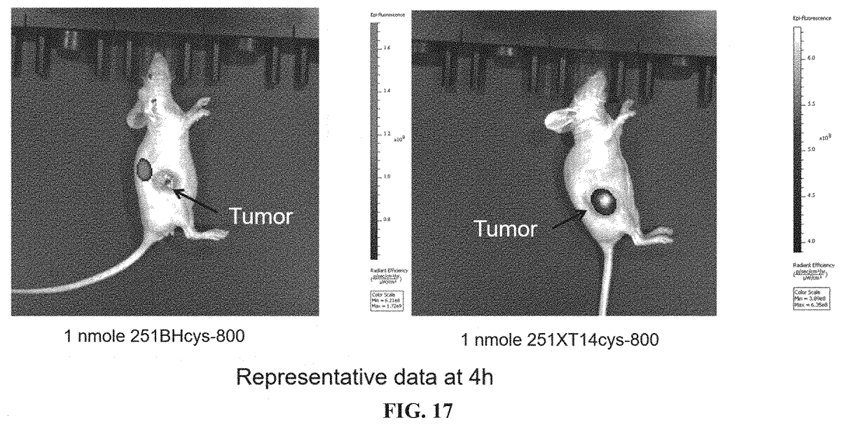

Briefly, human PD-L1 Fc (R&D Systems) chimeric protein was coated onto 96 well plates at 0.5 μg/mL in carbonate buffer. After saturation with 5% casein/PBS buffer, plates were washed and a dilution of conjugated AFFIMER® protein or unconjugated control were incubated for 90 minutes. Plates were then washed, a biotinylated polyclonal anti-cystatin A antibody (R&D Systems) added, and the plates incubated for 1 hour. Plates were washed and bound AFFIMER® protein was detected using streptavidin-HRP. After a last washing step, TMB was added and the plate was read at 450 nm. The conjugated AFFIMER® protein exhibited a similar EC₅₀ compared to the parental molecule. Therefore, the data indicate that dye conjugation does not impact the affinity of both conjugated formatted molecules for the PD-L1 target based on comparable binding curves (FIG. 16A-16B).

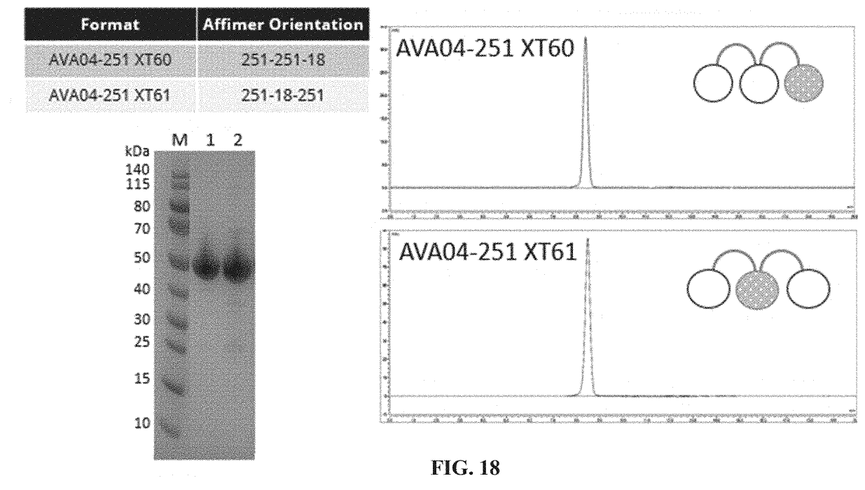

The A375 mouse xenograft model was established in female athymic nude mice (Charles River Laboratories) following subcutaneous injection of A375 cells (5×10⁶ cells [ATCC] in 100 μL sterile PBS) into the animal's flank. Tumors were monitored three (3) times per week, with the developing tumor being measured with calipers. Tumors were allowed to grow between 500-1000 mm³ prior to intravenous administration of AVA04-251 BQ-800 and BH-800 (at 1 nmole) into the tail vein of three (3) mice. Fluorescence images were recorded with a Xenogen IVIS 200 Biophotonic Imager immediately after injection (time 0) and at 1, 2, 4, 8, 24, and 48 hours post-dose. At the four (4) hour timepoint, targeting of the anti-PD-L1 AFFIMER® protein with half-life extension to the tumor was detected. The data are presented in FIG. 17, and arrows indicate the approximate locations of the tumor.

### Example 12: AVA04-251 XT ILF Formatting with HSA-18 Half-Life Extending AFFIMER® Polypeptides

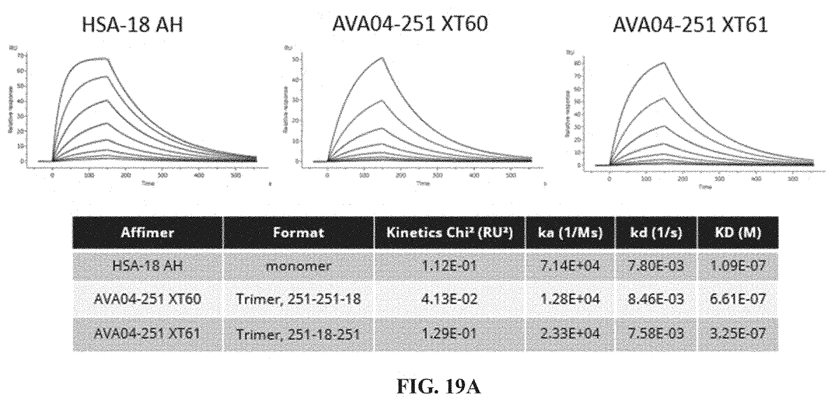

Two AFFIMER® trimeric in-line fusion (ILF) formats were designed. Each comprised two fused AVA04-251 human PD-L1 binding AFFIMER® polypeptides, which were further fused with an HSA AFFIMER® polypeptide (HSA-18) to extend half-life. AVA04-251 XT60 (SEQ ID NO. 290) comprised the half-life extending AFFIMER® protein positioned at the C-terminus, whereas AVA04-251 XT61 (SEQ ID NO. 291) comprised the half-life extension AFFIMER® polypeptide in the middle of the format, separating the two anti-PD-L1 AFFIMER® polypeptides (schematic diagrams, FIG. 18). Formats were designed with repetitive rigid genetic linkers A(EAAAK)₆ (SEQ ID NO: 294) between AFFIMER® polypeptides. AFFIMER® trimers were produced from E. coli and purified with affinity NiNTA resin followed by preparative size exclusion as described in Example 2. Reducing SDS-PAGE and SEC-HPLC analysis show the final purity of the protein formats was >98% (FIG. 18).

### Example 13: Half-Life Extended AVA04-251 XT60 and AVA04-251 XT61 ILF Format Binding to Serum Albumin

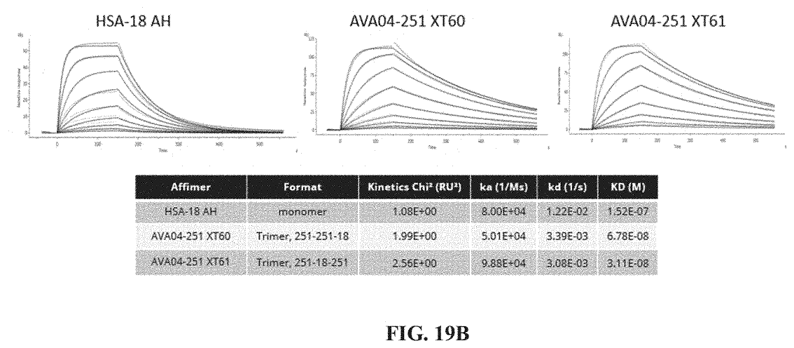

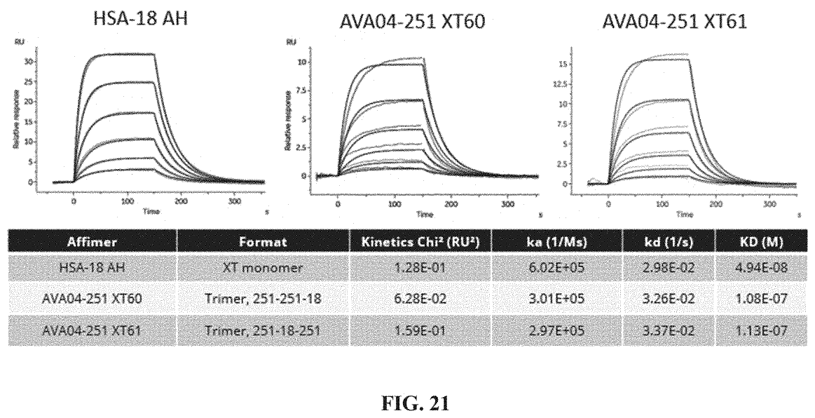

Human serum albumin (HSA) Biacore kinetic analysis was performed with pH6.0 and with pH7.4 running buffer using the method previously described in Example 3. Data showed the ILF formats containing a half-life extending HSA AFFIMER® polypeptide (HSA-18) bound HSA with a KD of triple digit nM affinity at pH7.4 and double digit nM affinity at pH6.0, within 2-4 fold of the HSA-18 monomer affinity of 109-152 nM (FIGS. 19A-19B). For mouse serum albumin (MSA) at pH6.0 conditions, binding affinity of the ILF formats was within approximately 2-fold of the monomer serum albumin binding AFFIMER® protein (FIG. 21).

### Example 14: Half-Life Extended AVA04-251 XT60 and AVA04-251 XT61 ILF Format Binding to Serum Albumin

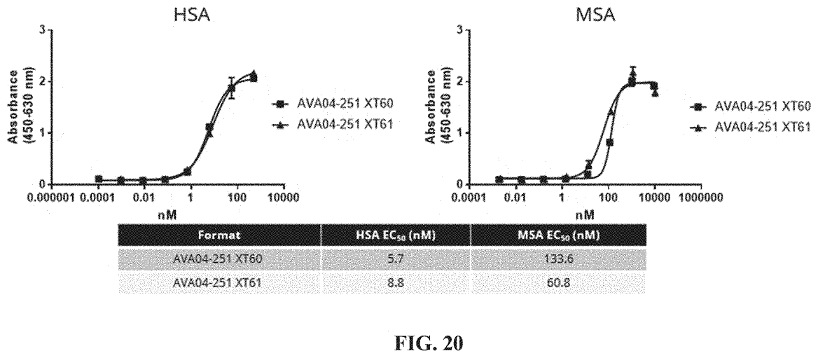

Binding to human serum albumin and mouse serum albumin was assessed for the two half-life extended ILF AFFIMER® formats (AVA04-251 XT60, SEQ ID NO: 290; AVA04-251 XT61, SEQ ID NO: 291) at pH 7.4 with an ELISA. Briefly, HSA or MSA was coated in 96 well plates at 1 mg/ml at pH 7.5. After saturation with 5% PBS Casein pH 7.5, plates were washed and a dilution of AFFIMER® trimers or controls were incubated on the plate for 90 minutes. Plates were then washed, and a biotinylated polyclonal antibody anti-cystatin A (R&D Systems) was added for 1 hour. Plates were washed and AFFIMER® ILFs were detected using streptavidin-HRP. After a last washing step, TMB was added for the development of the experiment, and the plates were read at 450 nm. The two ILFs tested, AVA04-251 XT60 and AVA04-251 XT61, exhibited similar EC₅₀ values for both HSA (ranging from 5.7 to 8.8) and MSA (ranging from 133.6 to 60.8) (FIG. 20).

### Example 15: AVA04-251 XT60 and AVA04-251 XT61 ILF Format Binding to Human PD-L1-Fc

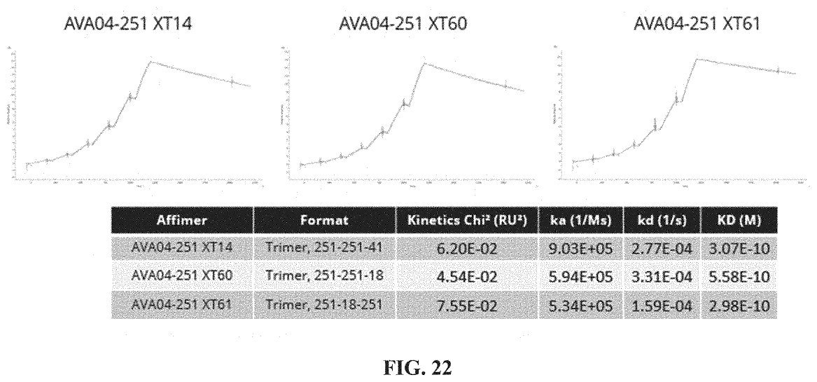

Biacore kinetic analysis was performed with single cycle kinetics to assess binding of AVA04-251 XT60 and AVA04-251 XT61 (SEQ ID NOs: 290 and 291 respectively) as described in Example 3. The experiments were performed to compare the AFFIMER® trimers to HSA-41. Binding affinity KD values were in the triple digit nM range, with similar on and off rates observed, regardless of whether the half-life extending AFFIMER® protein was in the middle or C-terminal end of the format (FIG. 22).

What is claimed is:

1. A fusion protein comprising a human serum albumin (HSA) binding recombinantly engineered variant of stefin polypeptide and a PD-L1 binding polypeptide, wherein the HSA binding polypeptide binds to HSA with a K_{d} of 1×10⁻⁶M or less at pH 6.0 and optionally a K_{d} for binding HSA at pH 7.4 that is at least half a log greater than the K_{d} for binding at pH 6.0, and wherein the PD-L1 binding polypeptide binds to PD-L1 with a K_{d} of 1×10⁻⁶M.

2. A fusion protein comprising an HSA binding recombinantly engineered variant of stefin polypeptide that binds to HSA and a PD-L1 binding recombinantly engineered variant of stefin polypeptide that binds to PD-L1, wherein the protein has a circulating half-life in human subjects of at least 7 days.

3. The fusion protein of claim 1 or 2, wherein the HSA binding polypeptide comprises a loop 2 sequences selected from any one of SEQ ID NOs: 86-138 and/or a loop 4 sequence selected from any one of SEQ ID NOs: 139-191.

4. The fusion protein of any one of claims 1-3, wherein the PD-L1 binding polypeptide comprises a loop 2 sequences selected from any one of SEQ ID NOs: 16-50 and/or a loop 4 sequence selected from any one of SEQ ID NOs: 51-85.

5. The fusion protein of any one of the preceeding claims, wherein the fusion protein comprises the amino acid sequence of SEQ ID NO: 277, or an amino acid sequence having at least 80%, at least 85%, at least 90%, or at least 95% identity to the amino acid sequence of SEQ ID NO: 277.

6. The fusion protein of any one of claims 1-5, wherein the fusion protein comprises the amino acid sequence of SEQ ID NO: 278, or an amino acid sequence having at least 80%, at least 85%, at least 90%, or at least 95% identity to the amino acid sequence of SEQ ID NO: 278.

7. The fusion protein of any one of claims 1-5, wherein the fusion protein comprises the amino acid sequence of SEQ ID NO: 279, or an amino acid sequence having at least 80%, at least 85%, at least 90%, or at least 95% identity to the amino acid sequence of SEQ ID NO: 279.

8. The fusion protein of any one of claims 1-5, wherein the fusion protein comprises the amino acid sequence of SEQ ID NO: 280, or an amino acid sequence having at least 80%, at least 85%, at least 90%, or at least 95% identity to the amino acid sequence of SEQ ID NO: 280.

9. The fusion protein of any one of claims 1-5, wherein the fusion protein comprises the amino acid sequence of SEQ ID NO: 281, or an amino acid sequence having at least 80%, at least 85%, at least 90%, or at least 95% identity to the amino acid sequence of SEQ ID NO: 281.

10. The fusion protein of any one of claims 1-5, wherein the fusion protein comprises the amino acid sequence of SEQ ID NO: 282, or an amino acid sequence having at least 80%, at least 85%, at least 90%, or at least 95% identity to the amino acid sequence of SEQ ID NO: 282.

11. The fusion protein of any one of claims 1-10 further comprising a second a PD-L1 binding polypeptide, wherein the second PD-L1 binding polypeptide binds to PD-L1 with a K_{d} of 1×10⁻⁶M.

12. An in-line fusion protein comprising a human serum albumin (HSA) binding recombinantly engineered variant of stefin, first a PD-L1 binding polypeptide, and a second a PD-L1 binding polypeptide, wherein the HSA binding polypeptide binds to HSA with a K_{d} of 1×10⁻⁶M or less at pH 6.0 and optionally a K_{d} for binding HSA at pH 7.4 that is at least half a log greater than the K_{d} for binding at pH 6.0, and wherein the first and second PD-L1 binding polypeptides bind to PD-L1 with a K_{d} of 1×10⁻⁶M.

13. The fusion protein of any one of the preceding claims, further comprising a linker.

14. The fusion protein of claim 13, wherein the linker is a rigid linker or a flexible linker.

15. The fusion protein of claim 14, wherein the rigid linker comprises the sequence of SEQ ID NO: 294, or the flexible linker comprises the sequence of SEQ ID NO: 293.

16. The fusion protein of any one of claims 13-15, wherein the fusion protein comprises the amino acid sequence of SEQ ID NO: 283, or an amino acid sequence having at least 80%, at least 85%, at least 90%, or at least 95% identity to the amino acid sequence of SEQ ID NO: 283.

17. The fusion protein of any one of claims 13-15, wherein the fusion protein comprises the amino acid sequence of SEQ ID NO: 284, or an amino acid sequence having at least 80%, at least 85%, at least 90%, or at least 95% identity to the amino acid sequence of SEQ ID NO: 284.

18. The fusion protein of any one of claims 13-15, wherein the fusion protein comprises the amino acid sequence of SEQ ID NO: 285, or an amino acid sequence having at least 80%, at least 85%, at least 90%, or at least 95% identity to the amino acid sequence of SEQ ID NO: 285.

19. The fusion protein of any one of claims 13-15, wherein the fusion protein comprises the amino acid sequence of SEQ ID NO: 286, or an amino acid sequence having at least 80%, at least 85%, at least 90%, or at least 95% identity to the amino acid sequence of SEQ ID NO: 286.

20. The fusion protein of any one of claims 13-15, wherein the fusion protein comprises the amino acid sequence of SEQ ID NO: 287, or an amino acid sequence having at least 80%, at least 85%, at least 90%, or at least 95% identity to the amino acid sequence of SEQ ID NO: 287.

21. The fusion protein of any one of claims 13-15, wherein the fusion protein comprises the amino acid sequence of SEQ ID NO: 290, or an amino acid sequence having at least 80%, at least 85%, at least 90%, or at least 95% identity to the amino acid sequence of SEQ ID NO: 290.

22. The fusion protein of any one of claims 13-15, wherein the fusion protein comprises the amino acid sequence of SEQ ID NO: 291, or an amino acid sequence having at least 80%, at least 85%, at least 90%, or at least 95% identity to the amino acid sequence of SEQ ID NO: 291.

23. The fusion protein of any one of claims 16-22, wherein the fusion protein further comprises a flexible linker, optionally wherein the flexible linker comprises the sequence of SEQ ID NO: 293.

24. A pharmaceutical composition suitable for therapeutic use in a human subject, comprising a fusion protein of any of any one of the preceding claims, and a pharmaceutically acceptable excipient.

25. A polynucleotide comprising a sequence encoding the fusion protein of any one of the claims 1-23.

26. The polynucleotide of claim 25, wherein the sequence encoding the fusion protein is operably linked to a transcriptional regulatory sequence.

27. A viral vector comprising the polynucleotide of claim 25 or 26.

28. A plasmid or minicircle comprising the polynucleotide any of claim 25 or 26.

29. A cell comprising the fusion protein of any one of claims 1-23, the polynucleotide of claim 25 or 26, the viral vector of claim 27, or the plasmid or minicircle of claim 28.

30. The fusion protein of any one of claims 1-23, for use in a method for treating an infectious disease.

31. The fusion protein of any one of claims 1-23, for use in a method for treating cancer.

32. The fusion protein of any one of claims 1-23, further comprising one or more additional polypeptide sequences which may additional therapeutic activity and/or additional PK/ADME properties to the fusion protein.

33. The fusion protein of claim 32, wherein the additional polypeptide sequence is a receptor ligand.

34. The fusion protein of claim 33, wherein the receptor ligand is an immunostimulatory cytokine, such as IFN-α, IL-2, IL-15, IL-21, and IL-12, or a variant sequence thereof.

35. The fusion protein of claim 33, wherein the additional polypeptide sequence is a mutant IL-2 polypeptide comprising the amino acid sequence of SEQ ID NO: 1282, or a sequence at least 70% identical thereof, having one or more amino acid substitutions relative to SEQ ID NO: 1282 that abolish or reduce affinity of the mutant IL-2 polypeptide to a high-affinity IL-2 receptor and preserve affinity of the mutant IL-2 polypeptide to an intermediate-affinity IL-2 receptor, as compared to a wild-type IL-2 polypeptide.

36. The fusion protein of claim 35, wherein the additional polypeptide sequence is a mutant IL-2 polypeptide comprising the amino acid sequence of SEQ ID NO: 1282 but having one or more amino acid substitutions at positions selected from T3 (such as T3A), D20 (such as D20T), R38 (such as R38A and R38D), F42 (such as F42A, F42G, F42S, F42T, F42Q, F42E, F42N, F42D, F42R or F42K), K43 (such as K43E), E61 (such as E61R), E62 (such as E62A), Y45 (such as Y45A, Y45G, Y45S, Y45T, Y45Q, Y45E, Y45N, Y45D, Y45R or Y45K) and/or L72 (such as L72G, L72A, L72S, L72T, L72Q, L72E, L72N, L72D, L72R or L72K).

37. The fusion protein of claim 33, wherein the receptor ligand is for a co-stimulatory receptor and the fusion protein agonizes the co-stimulatory receptor upon binding.

38. The fusion protein of claim 37, wherein the receptor ligand is selected from CD40L, CDB7.1, 4-1BBL, OX40L, GITRL or LIGHT.

39. The fusion protein of claim 33, further comprising a polypeptide sequences that induces dimerization or higher order multimerization of the fusion protein.

40. The fusion protein of claim 33, wherein the additional polypeptide sequence is a CD3 binding polypeptide sequence that directs the fusion protein to bind to CD3 on the surface of T-cells.


SEQUENCE LISTING (321 sequences)

SEQ ID NO: 1 - protein, 49 aa, Artificial Sequence
  Synthetic
MIPGGLSEAKPATPEIQEIVDKVKPQLEEKTNETYGKLEAVQYKTQVLA

SEQ ID NO: 2 - protein, 49 aa, Artificial Sequence
  Synthetic
MIPRGLSEAKPATPEIQEIVDKVKPQLEEKTNETYGKLEAVQYKTQVLA

SEQ ID NO: 3 - protein, 24 aa, Artificial Sequence
  Synthetic
GTNYYIKVRAGDNKYMHLKVFKSL

SEQ ID NO: 4 - protein, 24 aa, Artificial Sequence
  Synthetic
STNYYIKVRAGDNKYMHLKVFNGP

SEQ ID NO: 5 - protein, 21 aa, Artificial Sequence
  Synthetic
ADRVLTGYQVDKNKDDELTGF

SEQ ID NO: 6 - protein, 21 aa, Artificial Sequence
  Synthetic
EDLVLTGYQVDKNKDDELTGF

SEQ ID NO: 7 - protein, 136 aa, Artificial Sequence
  Synthetic
  SITE (4)..(4): Xaa may be Gly, Ala, Val, Arg, Lys, Asp, or Glu
  misc_feature (50)..(70): Xaa can be any naturally occurring amino acid
  MISC_FEATURE (53)..(70): May be absent
  SITE (71)..(71): Xaa may be Gly, Ala, Val, Ser or Thr
  SITE (92)..(92): Xaa may be Arg, Lys, Asn, Gln, Ser, or Thr
  SITE (93)..(93): Xaa may be Gly, Ala, Val, Ser or Thr
  SITE (94)..(94): Xaa may be Ala, Val, Ile, Leu, Gly or Pro
  misc_feature (95)..(115): Xaa can be any naturally occurring amino acid
  MISC_FEATURE (98)..(115): May be absent
  SITE (116)..(116): Xaa may be Gly, Ala, Val, Asp or Glu
  SITE (118)..(118): Xaa may be Ala, Val, Ile, Leu, Arg or Lys
MIPXGLSEAKPATPEIQEIVDKVKPQLEEKTNETYGKLEAVQYKTQVLAXXXXXXXXXXXXXXXXXXXXXXTNYYIKVRAGDNKYMHLKVFXXXXXXXXXXXXXXXXXXXXXXXXXDXVLTGYQVDKNKDDELTGF

SEQ ID NO: 8 - protein, 136 aa, Artificial Sequence
  Synthetic
  misc_feature (50)..(70): Xaa can be any naturally occurring amino acid
  MISC_FEATURE (53)..(70): May be absent
  misc_feature (95)..(115): Xaa can be any naturally occurring amino acid
  MISC_FEATURE (98)..(115): May be absent
MIPRGLSEAKPATPEIQEIVDKVKPQLEEKTNETYGKLEAVQYKTQVLAXXXXXXXXXXXXXXXXXXXXXSTNYYIKVRAGDNKYMHLKVFNGPXXXXXXXXXXXXXXXXXXXXXADRVLTGYQVDKNKDDELTGF

SEQ ID NO: 9 - protein, 136 aa, Artificial Sequence; identical to SEQ ID NO: 7
  Synthetic
  SITE (4)..(4): Xaa may be Gly, Ala, Val, Arg, Lys, Asp, or Glu
  misc_feature (50)..(70): Xaa can be any naturally occurring amino acid
  MISC_FEATURE (53)..(70): May be absent
  SITE (71)..(71): Xaa may be Gly, Ala, Val, Ser or Thr
  SITE (92)..(92): Xaa may be Arg, Lys, Asn, Gln, Ser, or Thr
  SITE (93)..(93): Xaa may be Gly, Ala, Val, Ser or Thr
  SITE (94)..(94): Xaa may be Ala, Val, Ile, Leu, Gly or Pro
  misc_feature (95)..(115): Xaa can be any naturally occurring amino acid
  MISC_FEATURE (98)..(115): May be absent
  SITE (116)..(116): Xaa may be Gly, Ala, Val, Asp or Glu
  SITE (118)..(118): Xaa may be Ala, Val, Ile, Leu, Arg or Lys

SEQ ID NO: 10 - protein, 136 aa, Artificial Sequence; identical to SEQ ID NO: 8
  Synthetic
  misc_feature (50)..(70): Xaa can be any naturally occurring amino acid
  MISC_FEATURE (53)..(70): May be absent
  misc_feature (95)..(115): Xaa can be any naturally occurring amino acid
  MISC_FEATURE (98)..(115): May be absent

SEQ ID NO: 11 - protein, 133 aa, Artificial Sequence
  Synthetic
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 12 - protein, 586 aa, Artificial Sequence
  Synthetic
MPLLLLLPLLWAGALAIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAAGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 13 - protein, 586 aa, Artificial Sequence
  Synthetic
MPLLLLLPLLWAGALAIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAAGGGGSGGGGSGGGGSGGGGSAPTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFAQSIISTLT

SEQ ID NO: 14 - protein, 586 aa, Artificial Sequence
  Synthetic
MPLLLLLPLLWAGALAIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAAGGGGSGGGGSGGGGSGGGGSAPTSSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFAQSIISTLT

SEQ ID NO: 15 - protein, 25 aa, Artificial Sequence
  Synthetic
  MISC_FEATURE (8)..(25): May be absent
SGGSGGSGGSGGSGGSGGSGGSGGS

SEQ ID NO: 16 - protein, 9 aa, Artificial Sequence
  Synthetic
KAWGPKQWW

SEQ ID NO: 17 - protein, 9 aa, Artificial Sequence
  Synthetic
KPYGPRDWD

SEQ ID NO: 18 - protein, 9 aa, Artificial Sequence
  Synthetic
KEYGPEEWW

SEQ ID NO: 19 - protein, 9 aa, Artificial Sequence
  Synthetic
HAYGPRDWD

SEQ ID NO: 20 - protein, 9 aa, Artificial Sequence
  Synthetic
KDHGPIAWW

SEQ ID NO: 21 - protein, 9 aa, Artificial Sequence
  Synthetic
NKHFHQRFW

SEQ ID NO: 22 - protein, 9 aa, Artificial Sequence
  Synthetic
NKHFPIHFW

SEQ ID NO: 23 - protein, 9 aa, Artificial Sequence
  Synthetic
HEFGPAEWD

SEQ ID NO: 24 - protein, 9 aa, Artificial Sequence
  Synthetic
NAHFPQSFW

SEQ ID NO: 25 - protein, 9 aa, Artificial Sequence
  Synthetic
KEHGPDSWW

SEQ ID NO: 26 - protein, 9 aa, Artificial Sequence
  Synthetic
NQHFPHSFW

SEQ ID NO: 27 - protein, 9 aa, Artificial Sequence
  Synthetic
NAHFGPRFW

SEQ ID NO: 28 - protein, 9 aa, Artificial Sequence
  Synthetic
NTWFPESFW

SEQ ID NO: 29 - protein, 9 aa, Artificial Sequence
  Synthetic
NQHFPQSFW

SEQ ID NO: 30 - protein, 9 aa, Artificial Sequence
  Synthetic
KQYGPDDWW

SEQ ID NO: 31 - protein, 9 aa, Artificial Sequence
  Synthetic
KDWGPSNWW

SEQ ID NO: 32 - protein, 9 aa, Artificial Sequence
  Synthetic
KQFGPKDWW

SEQ ID NO: 33 - protein, 9 aa, Artificial Sequence
  Synthetic
NHHFPKRFW

SEQ ID NO: 34 - protein, 8 aa, Artificial Sequence
  Synthetic
YRHFPQWH

SEQ ID NO: 35 - protein, 9 aa, Artificial Sequence
  Synthetic
NIHFPPNFW

SEQ ID NO: 36 - protein, 8 aa, Artificial Sequence
  Synthetic
YTHFPQWT

SEQ ID NO: 37 - protein, 9 aa, Artificial Sequence
  Synthetic
NDHFPHTFW

SEQ ID NO: 38 - protein, 9 aa, Artificial Sequence
  Synthetic
NQHFPSYFW

SEQ ID NO: 39 - protein, 9 aa, Artificial Sequence
  Synthetic
NQYFPPHFW

SEQ ID NO: 40 - protein, 9 aa, Artificial Sequence
  Synthetic
KKHFPASFW

SEQ ID NO: 41 - protein, 9 aa, Artificial Sequence
  Synthetic
KKFFPKHFW

SEQ ID NO: 42 - protein, 9 aa, Artificial Sequence
  Synthetic
KLHFPRSFW

SEQ ID NO: 43 - protein, 9 aa, Artificial Sequence
  Synthetic
YKHFPPNFW

SEQ ID NO: 44 - protein, 9 aa, Artificial Sequence
  Synthetic
EEHFPFQFW

SEQ ID NO: 45 - protein, 9 aa, Artificial Sequence
  Synthetic
KPHFPDNFW

SEQ ID NO: 46 - protein, 9 aa, Artificial Sequence
  Synthetic
YQYFPDQFN

SEQ ID NO: 47 - protein, 9 aa, Artificial Sequence
  Synthetic
VQWFPRSFW

SEQ ID NO: 48 - protein, 9 aa, Artificial Sequence
  Synthetic
AAHFPEHFW

SEQ ID NO: 49 - protein, 9 aa, Artificial Sequence
  Synthetic
REGRQDWVL

SEQ ID NO: 50 - protein, 9 aa, Artificial Sequence
  Synthetic
WVPFPHQQL

SEQ ID NO: 51 - protein, 5 aa, Artificial Sequence
  Synthetic
GRTIQ

SEQ ID NO: 52 - protein, 9 aa, Artificial Sequence
  Synthetic
EPQLDTSPI

SEQ ID NO: 53 - protein, 9 aa, Artificial Sequence
  Synthetic
GDYEQVLIH

SEQ ID NO: 54 - protein, 9 aa, Artificial Sequence
  Synthetic
PADHVLEEA

SEQ ID NO: 55 - protein, 9 aa, Artificial Sequence
  Synthetic
EDTNTDGAL

SEQ ID NO: 56 - protein, 9 aa, Artificial Sequence
  Synthetic
GQSWDQRRQ

SEQ ID NO: 57 - protein, 9 aa, Artificial Sequence
  Synthetic
SKSPIDLPF

SEQ ID NO: 58 - protein, 9 aa, Artificial Sequence
  Synthetic
DPQDVYLNQ

SEQ ID NO: 59 - protein, 9 aa, Artificial Sequence
  Synthetic
GSLHSFGST

SEQ ID NO: 60 - protein, 9 aa, Artificial Sequence
  Synthetic
QEKNQWVEE

SEQ ID NO: 61 - protein, 9 aa, Artificial Sequence
  Synthetic
QKNYEEDPH

SEQ ID NO: 62 - protein, 9 aa, Artificial Sequence
  Synthetic
WDGHKRFAD

SEQ ID NO: 63 - protein, 9 aa, Artificial Sequence
  Synthetic
DDNQERQEH

SEQ ID NO: 64 - protein, 9 aa, Artificial Sequence
  Synthetic
AVTQEDQAV

SEQ ID NO: 65 - protein, 9 aa, Artificial Sequence
  Synthetic
EVDWKYQDH

SEQ ID NO: 66 - protein, 9 aa, Artificial Sequence
  Synthetic
VDDKTLSKD

SEQ ID NO: 67 - protein, 9 aa, Artificial Sequence
  Synthetic
QGQGKDPSQ

SEQ ID NO: 68 - protein, 9 aa, Artificial Sequence
  Synthetic
GHQSEVQHS

SEQ ID NO: 69 - protein, 9 aa, Artificial Sequence
  Synthetic
TGTSIWNQD

SEQ ID NO: 70 - protein, 11 aa, Artificial Sequence
  Synthetic
GVHDSLQGYDA

SEQ ID NO: 71 - protein, 9 aa, Artificial Sequence
  Synthetic
QKGQKIDKF

SEQ ID NO: 72 - protein, 9 aa, Artificial Sequence
  Synthetic
DDELHDTRH

SEQ ID NO: 73 - protein, 9 aa, Artificial Sequence
  Synthetic
ATTGDEWDR

SEQ ID NO: 74 - protein, 9 aa, Artificial Sequence
  Synthetic
SHPHSNHTS

SEQ ID NO: 75 - protein, 9 aa, Artificial Sequence
  Synthetic
WRTDYKYEE

SEQ ID NO: 76 - protein, 9 aa, Artificial Sequence
  Synthetic
NDPHDSVPH

SEQ ID NO: 77 - protein, 9 aa, Artificial Sequence
  Synthetic
GQQRENEQE

SEQ ID NO: 78 - protein, 9 aa, Artificial Sequence
  Synthetic
GERQQDDAN

SEQ ID NO: 79 - protein, 9 aa, Artificial Sequence
  Synthetic
AYREGSQWT

SEQ ID NO: 80 - protein, 9 aa, Artificial Sequence
  Synthetic
EFYDHGIIQ

SEQ ID NO: 81 - protein, 9 aa, Artificial Sequence
  Synthetic
ENEATRDQH

SEQ ID NO: 82 - protein, 9 aa, Artificial Sequence
  Synthetic
GYDHEDNRG

SEQ ID NO: 83 - protein, 9 aa, Artificial Sequence
  Synthetic
QPADMSAEF

SEQ ID NO: 84 - protein, 9 aa, Artificial Sequence
  Synthetic
WVPFPHQQL

SEQ ID NO: 85 - protein, 9 aa, Artificial Sequence
  Synthetic
REGRQDWVL

SEQ ID NO: 86 - protein, 9 aa, Artificial Sequence
  Synthetic
WTQPKNEHH

SEQ ID NO: 87 - protein, 9 aa, Artificial Sequence
  Synthetic
HLKHTDAQP

SEQ ID NO: 88 - protein, 9 aa, Artificial Sequence
  Synthetic
HDQDVLHAW

SEQ ID NO: 89 - protein, 9 aa, Artificial Sequence
  Synthetic
KFHRQEWAD

SEQ ID NO: 90 - protein, 9 aa, Artificial Sequence
  Synthetic
PEDFWDPEH

SEQ ID NO: 91 - protein, 9 aa, Artificial Sequence
  Synthetic
VVRTTGHVV

SEQ ID NO: 92 - protein, 9 aa, Artificial Sequence
  Synthetic
YWWFCTGQS

SEQ ID NO: 93 - protein, 9 aa, Artificial Sequence
  Synthetic
IHHRQARSL

SEQ ID NO: 94 - protein, 9 aa, Artificial Sequence
  Synthetic
SHRRRAYIW

SEQ ID NO: 95 - protein, 9 aa, Artificial Sequence
  Synthetic
WDSHHWRAP

SEQ ID NO: 96 - protein, 8 aa, Artificial Sequence
  Synthetic
DKRVKYGQ

SEQ ID NO: 97 - protein, 9 aa, Artificial Sequence
  Synthetic
SDWVYALQL

SEQ ID NO: 98 - protein, 6 aa, Artificial Sequence
  Synthetic
FWWFWY

SEQ ID NO: 99 - protein, 9 aa, Artificial Sequence
  Synthetic
VRDWPWNTF

SEQ ID NO: 100 - protein, 9 aa, Artificial Sequence
  Synthetic
QKKRDEDYI

SEQ ID NO: 101 - protein, 9 aa, Artificial Sequence
  Synthetic
GVHEEPRKL

SEQ ID NO: 102 - protein, 9 aa, Artificial Sequence
  Synthetic
EWWQKHWPS

SEQ ID NO: 103 - protein, 9 aa, Artificial Sequence
  Synthetic
NFFQRRWPG

SEQ ID NO: 104 - protein, 9 aa, Artificial Sequence
  Synthetic
DWWQAKWPH

SEQ ID NO: 105 - protein, 9 aa, Artificial Sequence
  Synthetic
GIWQSRWPG

SEQ ID NO: 106 - protein, 9 aa, Artificial Sequence
  Synthetic
GYWAAKWPG

SEQ ID NO: 107 - protein, 9 aa, Artificial Sequence
  Synthetic
GFYADHWPG

SEQ ID NO: 108 - protein, 9 aa, Artificial Sequence
  Synthetic
NWYQQRWPG

SEQ ID NO: 109 - protein, 9 aa, Artificial Sequence
  Synthetic
GFYARHWPG

SEQ ID NO: 110 - protein, 9 aa, Artificial Sequence
  Synthetic
DFWKAHWPG

SEQ ID NO: 111 - protein, 9 aa, Artificial Sequence
  Synthetic
DFYSVRWPG

SEQ ID NO: 112 - protein, 9 aa, Artificial Sequence
  Synthetic
YWAANHASK

SEQ ID NO: 113 - protein, 9 aa, Artificial Sequence
  Synthetic
IKRLEHWEY

SEQ ID NO: 114 - protein, 9 aa, Artificial Sequence
  Synthetic
EWDSPWSEN

SEQ ID NO: 115 - protein, 9 aa, Artificial Sequence
  Synthetic
KHKNLRWPF

SEQ ID NO: 116 - protein, 9 aa, Artificial Sequence
  Synthetic
RHFPKQTNW

SEQ ID NO: 117 - protein, 9 aa, Artificial Sequence
  Synthetic
VWGPEYQHQ

SEQ ID NO: 118 - protein, 9 aa, Artificial Sequence
  Synthetic
TWKNNGQDV

SEQ ID NO: 119 - protein, 9 aa, Artificial Sequence
  Synthetic
ATWLNYYLP

SEQ ID NO: 120 - protein, 9 aa, Artificial Sequence
  Synthetic
DQESLFLNN

SEQ ID NO: 121 - protein, 9 aa, Artificial Sequence
  Synthetic
GFYAQHWPD

SEQ ID NO: 122 - protein, 9 aa, Artificial Sequence
  Synthetic
GHYARYWPG

SEQ ID NO: 123 - protein, 9 aa, Artificial Sequence
  Synthetic
GFWASKWPG

SEQ ID NO: 124 - protein, 9 aa, Artificial Sequence
  Synthetic
GFWQRKWPN

SEQ ID NO: 125 - protein, 9 aa, Artificial Sequence
  Synthetic
VWPADNDLK

SEQ ID NO: 126 - protein, 9 aa, Artificial Sequence
  Synthetic
HWAWTSPGY

SEQ ID NO: 127 - protein, 9 aa, Artificial Sequence
  Synthetic
NFFQRRWPG

SEQ ID NO: 128 - protein, 9 aa, Artificial Sequence
  Synthetic
HHSHRLKGQ

SEQ ID NO: 129 - protein, 9 aa, Artificial Sequence
  Synthetic
YQNTIFLSI

SEQ ID NO: 130 - protein, 9 aa, Artificial Sequence
  Synthetic
FQDQFTWSQ

SEQ ID NO: 131 - protein, 9 aa, Artificial Sequence
  Synthetic
GEPHWPWQA

SEQ ID NO: 132 - protein, 9 aa, Artificial Sequence
  Synthetic
ADPRHPWVE

SEQ ID NO: 133 - protein, 9 aa, Artificial Sequence
  Synthetic
FHKRFQSQG

SEQ ID NO: 134 - protein, 9 aa, Artificial Sequence
  Synthetic
EWWQNRWPN

SEQ ID NO: 135 - protein, 9 aa, Artificial Sequence
  Synthetic
EWYQTRWPG

SEQ ID NO: 136 - protein, 9 aa, Artificial Sequence
  Synthetic
EFWQRHWPG

SEQ ID NO: 137 - protein, 9 aa, Artificial Sequence
  Synthetic
KFYERHWPG

SEQ ID NO: 138 - protein, 9 aa, Artificial Sequence
  Synthetic
GWWQRRWPG

SEQ ID NO: 139 - protein, 9 aa, Artificial Sequence
  Synthetic
RFKYFAHYQ

SEQ ID NO: 140 - protein, 9 aa, Artificial Sequence
  Synthetic
FHDFWHRRW

SEQ ID NO: 141 - protein, 9 aa, Artificial Sequence
  Synthetic
DWYHYWWEV

SEQ ID NO: 142 - protein, 9 aa, Artificial Sequence
  Synthetic
STRSIHVTT

SEQ ID NO: 143 - protein, 9 aa, Artificial Sequence
  Synthetic
KQHHHYLDK

SEQ ID NO: 144 - protein, 9 aa, Artificial Sequence
  Synthetic
HSAQDREIP

SEQ ID NO: 145 - protein, 9 aa, Artificial Sequence
  Synthetic
WVQSGYNSQ

SEQ ID NO: 146 - protein, 9 aa, Artificial Sequence
  Synthetic
AVFWGKWSD

SEQ ID NO: 147 - protein, 9 aa, Artificial Sequence
  Synthetic
QSFDKPWTT

SEQ ID NO: 148 - protein, 9 aa, Artificial Sequence
  Synthetic
HYPLKYSFE

SEQ ID NO: 149 - protein, 9 aa, Artificial Sequence
  Synthetic
WHHPWHRNR

SEQ ID NO: 150 - protein, 9 aa, Artificial Sequence
  Synthetic
DPWWAWVVW

SEQ ID NO: 151 - protein, 9 aa, Artificial Sequence
  Synthetic
FDNQDLIQY

SEQ ID NO: 152 - protein, 9 aa, Artificial Sequence
  Synthetic
EKKNWYKWD

SEQ ID NO: 153 - protein, 9 aa, Artificial Sequence
  Synthetic
DRHKSRWGI

SEQ ID NO: 154 - protein, 9 aa, Artificial Sequence
  Synthetic
LNPFTPSVT

SEQ ID NO: 155 - protein, 9 aa, Artificial Sequence
  Synthetic
YKGALLNHD

SEQ ID NO: 156 - protein, 9 aa, Artificial Sequence
  Synthetic
WKFRNTERG

SEQ ID NO: 157 - protein, 9 aa, Artificial Sequence
  Synthetic
YKVHQSSGG

SEQ ID NO: 158 - protein, 9 aa, Artificial Sequence
  Synthetic
FHPIAGRPW

SEQ ID NO: 159 - protein, 9 aa, Artificial Sequence
  Synthetic
FPNTSYDLQ

SEQ ID NO: 160 - protein, 9 aa, Artificial Sequence
  Synthetic
FAHYNLKSG

SEQ ID NO: 161 - protein, 9 aa, Artificial Sequence
  Synthetic
WHNYGESSG

SEQ ID NO: 162 - protein, 9 aa, Artificial Sequence
  Synthetic
KFYYADHQW

SEQ ID NO: 163 - protein, 9 aa, Artificial Sequence
  Synthetic
YTHADPHSQ

SEQ ID NO: 164 - protein, 9 aa, Artificial Sequence
  Synthetic
FGVPQLGAG

SEQ ID NO: 165 - protein, 9 aa, Artificial Sequence
  Synthetic
YSGFPFAGF

SEQ ID NO: 166 - protein, 9 aa, Artificial Sequence
  Synthetic
WFSWPYTPL

SEQ ID NO: 167 - protein, 9 aa, Artificial Sequence
  Synthetic
YYHPSIQST

SEQ ID NO: 168 - protein, 9 aa, Artificial Sequence
  Synthetic
FLGWKDTVV

SEQ ID NO: 169 - protein, 9 aa, Artificial Sequence
  Synthetic
DWWKWWWAK

SEQ ID NO: 170 - protein, 9 aa, Artificial Sequence
  Synthetic
NAGWPLVPE

SEQ ID NO: 171 - protein, 9 aa, Artificial Sequence
  Synthetic
YALDPFGGK

SEQ ID NO: 172 - protein, 9 aa, Artificial Sequence
  Synthetic
GYKFWGVSD

SEQ ID NO: 173 - protein, 9 aa, Artificial Sequence
  Synthetic
QGKQYILLR

SEQ ID NO: 174 - protein, 9 aa, Artificial Sequence
  Synthetic
YKRHSAHDY

SEQ ID NO: 175 - protein, 9 aa, Artificial Sequence
  Synthetic
WAQKSKVHQ

SEQ ID NO: 176 - protein, 9 aa, Artificial Sequence
  Synthetic
FTAVSKKDA

SEQ ID NO: 177 - protein, 9 aa, Artificial Sequence
  Synthetic
WGDKENIWF

SEQ ID NO: 178 - protein, 9 aa, Artificial Sequence
  Synthetic
WSGHPWVQK

SEQ ID NO: 179 - protein, 9 aa, Artificial Sequence
  Synthetic
YADYPLSPK

SEQ ID NO: 180 - protein, 9 aa, Artificial Sequence
  Synthetic
WKFRNTDRG

SEQ ID NO: 181 - protein, 9 aa, Artificial Sequence
  Synthetic
QTVATHYHY

SEQ ID NO: 182 - protein, 9 aa, Artificial Sequence
  Synthetic
WHAKHLLSH

SEQ ID NO: 183 - protein, 9 aa, Artificial Sequence
  Synthetic
SGIKKADSV

SEQ ID NO: 184 - protein, 9 aa, Artificial Sequence
  Synthetic
KANLINVKS

SEQ ID NO: 185 - protein, 9 aa, Artificial Sequence
  Synthetic
WKSHVEVRS

SEQ ID NO: 186 - protein, 9 aa, Artificial Sequence
  Synthetic
WVTQKYIIQ

SEQ ID NO: 187 - protein, 9 aa, Artificial Sequence
  Synthetic
WEHAKDWPT

SEQ ID NO: 188 - protein, 9 aa, Artificial Sequence
  Synthetic
FHSKVLDKA

SEQ ID NO: 189 - protein, 9 aa, Artificial Sequence
  Synthetic
YGAQKQAVW

SEQ ID NO: 190 - protein, 9 aa, Artificial Sequence
  Synthetic
FSASHFTSQ

SEQ ID NO: 191 - protein, 9 aa, Artificial Sequence
  Synthetic
  misc_feature (1)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
XXAXKXDXQ

SEQ ID NO: 192 - protein, 112 aa, Artificial Sequence
  Synthetic
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGF

SEQ ID NO: 193 - protein, 112 aa, Artificial Sequence
  Synthetic
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGF

SEQ ID NO: 194 - protein, 112 aa, Artificial Sequence
  Synthetic
MIPRGLSEAKPATPEIQEIVDKVKPQLEEKTNETYGKLEAVQYKTQVLAKDHGPIAWWSTNYYIKVRAGDNKYMHLKVFNGPEDTNTDGALADRVLTGYQVDKNKDDELTGF

SEQ ID NO: 195 - protein, 112 aa, Artificial Sequence
  Synthetic
MIPRGLSEAKPATPEIQEIVDKVKPQLEEKTNETYGKLEAVQYKTQVLAKDWGPSNWWSTNYYIKVRAGDNKYMHLKVFNGPVDDKTLSKDADRVLTGYQVDKNKDDELTGF

SEQ ID NO: 196 - protein, 112 aa, Artificial Sequence
  Synthetic
MIPRGLSEAKPATPEIQEIVDKVKPQLEEKTNETYGKLEAVQYKTQVLANTWFPESFWSTNYYIKVRAGDNKYMHLKVFNGPDDNQERQEHADRVLTGYQVDKNKDDELTGF

SEQ ID NO: 197 - protein, 112 aa, Artificial Sequence
  Synthetic
MIPRGLSEAKPATPEIQEIVDKVKPQLEEKTNETYGKLEAVQYKTQVLAKPYGPRDWDSTNYYIKVRAGDNKYMHLKVFNGPEPQLDTSPIADRVLTGYQVDKNKDDELTGF

SEQ ID NO: 198 - protein, 112 aa, Artificial Sequence
  Synthetic
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGF

SEQ ID NO: 199 - protein, 112 aa, Artificial Sequence
  Synthetic
MIPRGLSEAKPATPEIQEIVDKVKPQLEEKTNETYGKLEAVQYKTQVLAAAHFPEHFWSTNYYIKVRAGDNKYMHLKVFNGPQPADMSAEFADRVLTGYQVDKNKDDELTGF

SEQ ID NO: 200 - protein, 112 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 201 - protein, 112 aa, Artificial Sequence
  Synthetic
MIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 202 - protein, 112 aa, Artificial Sequence
  Synthetic
MIPRGLSEAKPATPEIQEIVDKVKPQLEEKTNETYGKLEAVQYKTQVLAGIWQSRWPGSTNYYIKVRAGDNKYMHLKVFNGPFHPIAGRPWADRVLTGYQVDKNKDDELTGF

SEQ ID NO: 203 - protein, 112 aa, Artificial Sequence
  Synthetic
MIPRGLSEAKPATPEIQEIVDKVKPQLEEKTNETYGKLEAVQYKTQVLAGYWAAKWPGSTNYYIKVRAGDNKYMHLKVFNGPFPNTSYDLQADRVLTGYQVDKNKDDELTGF

SEQ ID NO: 204 - protein, 112 aa, Artificial Sequence
  Synthetic
MIPRGLSEAKPATPEIQEIVDKVKPQLEEKTNETYGKLEAVQYKTQVLAGFYADHWPGSTNYYIKVRAGDNKYMHLKVFNGPFAHYNLKSGADRVLTGYQVDKNKDDELTGF

SEQ ID NO: 205 - protein, 112 aa, Artificial Sequence
  Synthetic
MIPRGLSEAKPATPEIQEIVDKVKPQLEEKTNETYGKLEAVQYKTQVLANWYQQRWPGSTNYYIKVRAGDNKYMHLKVFNGPWHNYGESSGADRVLTGYQVDKNKDDELTGF

SEQ ID NO: 206 - protein, 112 aa, Artificial Sequence
  Synthetic
MIPRGLSEAKPATPEIQEIVDKVKPQLEEKTNETYGKLEAVQYKTQVLAGFYARHWPGSTNYYIKVRAGDNKYMHLKVFNGPKFYYADHQWADRVLTGYQVDKNKDDELTGF

SEQ ID NO: 207 - protein, 112 aa, Artificial Sequence
  Synthetic
MIPRGLSEAKPATPEIQEIVDKVKPQLEEKTNETYGKLEAVQYKTQVLADFWKAHWPGSTNYYIKVRAGDNKYMHLKVFNGPYTHADPHSQADRVLTGYQVDKNKDDELTGF

SEQ ID NO: 208 - protein, 112 aa, Artificial Sequence
  Synthetic
MIPRGLSEAKPATPEIQEIVDKVKPQLEEKTNETYGKLEAVQYKTQVLADFYSVRWPGSTNYYIKVRAGDNKYMHLKVFNGPFGVPQLGAGADRVLTGYQVDKNKDDELTGF

SEQ ID NO: 209 - protein, 112 aa, Artificial Sequence
  Synthetic
MIPRGLSEAKPATPEIQEIVDKVKPQLEEKTNETYGKLEAVQYKTQVLAYWAANHASKSTNYYIKVRAGDNKYMHLKVFNGPYSGFPFAGFADRVLTGYQVDKNKDDELTGF

SEQ ID NO: 210 - protein, 112 aa, Artificial Sequence
  Synthetic
MIPRGLSEAKPATPEIQEIVDKVKPQLEEKTNETYGKLEAVQYKTQVLAIKRLEHWEYSTNYYIKVRAGDNKYMHLKVFNGPWFSWPYTPLADRVLTGYQVDKNKDDELTGF

SEQ ID NO: 211 - protein, 112 aa, Artificial Sequence
  Synthetic
MIPRGLSEAKPATPEIQEIVDKVKPQLEEKTNETYGKLEAVQYKTQVLAEWDSPWSENSTNYYIKVRAGDNKYMHLKVFNGPYYHPSIQSTADRVLTGYQVDKNKDDELTGF

SEQ ID NO: 212 - protein, 112 aa, Artificial Sequence
  Synthetic
MIPRGLSEAKPATPEIQEIVDKVKPQLEEKTNETYGKLEAVQYKTQVLAKHKNLRWPFSTNYYIKVRAGDNKYMHLKVFNGPFLGWKDTVVADRVLTGYQVDKNKDDELTGF

SEQ ID NO: 213 - protein, 112 aa, Artificial Sequence
  Synthetic
MIPRGLSEAKPATPEIQEIVDKVKPQLEEKTNETYGKLEAVQYKTQVLARHFPKQTNWSTNYYIKVRAGDNKYMHLKVFNGPDWWKWWWAKADRVLTGYQVDKNKDDELTGF

SEQ ID NO: 214 - protein, 112 aa, Artificial Sequence
  Synthetic
MIPRGLSEAKPATPEIQEIVDKVKPQLEEKTNETYGKLEAVQYKTQVLAVWGPEYQHQSTNYYIKVRAGDNKYMHLKVFNGPNAGWPLVPEADRVLTGYQVDKNKDDELTGF

SEQ ID NO: 215 - protein, 112 aa, Artificial Sequence
  Synthetic
MIPRGLSEAKPATPEIQEIVDKVKPQLEEKTNETYGKLEAVQYKTQVLATWKNNGQDVSTNYYIKVRAGDNKYMHLKVFNGPYALDPFGGKADRVLTGYQVDKNKDDELTGF

SEQ ID NO: 216 - protein, 112 aa, Artificial Sequence
  Synthetic
MIPRGLSEAKPATPEIQEIVDKVKPQLEEKTNETYGKLEAVQYKTQVLAATWLNYYLPSTNYYIKVRAGDNKYMHLKVFNGPGYKFWGVSDADRVLTGYQVDKNKDDELTGF

SEQ ID NO: 217 - protein, 112 aa, Artificial Sequence
  Synthetic
MIPRGLSEAKPATPEIQEIVDKVKPQLEEKTNETYGKLEAVQYKTQVLADQESLFLNNSTNYYIKVRAGDNKYMHLKVFNGPQGKQYILLRADRVLTGYQVDKNKDDELTGF

SEQ ID NO: 218 - protein, 112 aa, Artificial Sequence
  Synthetic
MIPRGLSEAKPATPEIQEIVDKVKPQLEEKTNETYGKLEAVQYKTQVLAGFYAQHWPDSTNYYIKVRAGDNKYMHLKVFNGPYKRHSAHDYADRVLTGYQVDKNKDDELTGF

SEQ ID NO: 219 - protein, 112 aa, Artificial Sequence
  Synthetic
MIPRGLSEAKPATPEIQEIVDKVKPQLEEKTNETYGKLEAVQYKTQVLAGHYARYWPGSTNYYIKVRAGDNKYMHLKVFNGPWAQKSKVHQADRVLTGYQVDKNKDDELTGF

SEQ ID NO: 220 - protein, 112 aa, Artificial Sequence
  Synthetic
MIPRGLSEAKPATPEIQEIVDKVKPQLEEKTNETYGKLEAVQYKTQVLAGFWASKWPGSTNYYIKVRAGDNKYMHLKVFNGPFTAVSKKDAADRVLTGYQVDKNKDDELTGF

SEQ ID NO: 221 - protein, 112 aa, Artificial Sequence
  Synthetic
MIPRGLSEAKPATPEIQEIVDKVKPQLEEKTNETYGKLEAVQYKTQVLAGFWQRKWPNSTNYYIKVRAGDNKYMHLKVFNGPWGDKENIWFADRVLTGYQVDKNKDDELTGF

SEQ ID NO: 222 - protein, 112 aa, Artificial Sequence
  Synthetic
MIPRGLSEAKPATPEIQEIVDKVKPQLEEKTNETYGKLEAVQYKTQVLAVWPADNDLKSTNYYIKVRAGDNKYMHLKVFNGPWSGHPWVQKADRVLTGYQVDKNKDDELTGF

SEQ ID NO: 223 - protein, 112 aa, Artificial Sequence
  Synthetic
MIPRGLSEAKPATPEIQEIVDKVKPQLEEKTNETYGKLEAVQYKTQVLAHWAWTSPGYSTNYYIKVRAGDNKYMHLKVFNGPYADYPLSPKADRVLTGYQVDKNKDDELTGF

SEQ ID NO: 224 - protein, 112 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 225 - protein, 112 aa, Artificial Sequence
  Synthetic
MIPRGLSEAKPATPEIQEIVDKVKPQLEEKTNETYGKLEAVQYKTQVLAHHSHRLKGQSTNYYIKVRAGDNKYMHLKVFNGPQTVATHYHYADRVLTGYQVDKNKDDELTGF

SEQ ID NO: 226 - protein, 112 aa, Artificial Sequence
  Synthetic
MIPRGLSEAKPATPEIQEIVDKVKPQLEEKTNETYGKLEAVQYKTQVLAYQNTIFLSISTNYYIKVRAGDNKYMHLKVFNGPWHAKHLLSHADRVLTGYQVDKNKDDELTGF

SEQ ID NO: 227 - protein, 112 aa, Artificial Sequence
  Synthetic
MIPRGLSEAKPATPEIQEIVDKVKPQLEEKTNETYGKLEAVQYKTQVLAFQDQFTWSQSTNYYIKVRAGDNKYMHLKVFNGPSGIKKADSVADRVLTGYQVDKNKDDELTGF

SEQ ID NO: 228 - protein, 112 aa, Artificial Sequence
  Synthetic
MIPRGLSEAKPATPEIQEIVDKVKPQLEEKTNETYGKLEAVQYKTQVLAGEPHWPWQASTNYYIKVRAGDNKYMHLKVFNGPKANLINVKSADRVLTGYQVDKNKDDELTGF

SEQ ID NO: 229 - protein, 112 aa, Artificial Sequence
  Synthetic
MIPRGLSEAKPATPEIQEIVDKVKPQLEEKTNETYGKLEAVQYKTQVLAADPRHPWVESTNYYIKVRAGDNKYMHLKVFNGPWKSHVEVRSADRVLTGYQVDKNKDDELTGF

SEQ ID NO: 230 - protein, 112 aa, Artificial Sequence
  Synthetic
MIPRGLSEAKPATPEIQEIVDKVKPQLEEKTNETYGKLEAVQYKTQVLAFHKRFQSQGSTNYYIKVRAGDNKYMHLKVFNGPWVTQKYIIQADRVLTGYQVDKNKDDELTGF

SEQ ID NO: 231 - protein, 112 aa, Artificial Sequence
  Synthetic
MIPRGLSEAKPATPEIQEIVDKVKPQLEEKTNETYGKLEAVQYKTQVLAEWWQNRWPNSTNYYIKVRAGDNKYMHLKVFNGPWEHAKDWPTADRVLTGYQVDKNKDDELTGF

SEQ ID NO: 232 - protein, 112 aa, Artificial Sequence
  Synthetic
MIPRGLSEAKPATPEIQEIVDKVKPQLEEKTNETYGKLEAVQYKTQVLAEWYQTRWPGSTNYYIKVRAGDNKYMHLKVFNGPFHSKVLDKAADRVLTGYQVDKNKDDELTGF

SEQ ID NO: 233 - protein, 112 aa, Artificial Sequence
  Synthetic
MIPRGLSEAKPATPEIQEIVDKVKPQLEEKTNETYGKLEAVQYKTQVLAEFWQRHWPGSTNYYIKVRAGDNKYMHLKVFNGPYGAQKQAVWADRVLTGYQVDKNKDDELTGF

SEQ ID NO: 234 - protein, 112 aa, Artificial Sequence
  Synthetic
MIPRGLSEAKPATPEIQEIVDKVKPQLEEKTNETYGKLEAVQYKTQVLAKFYERHWPGSTNYYIKVRAGDNKYMHLKVFNGPFSASHFTSQADRVLTGYQVDKNKDDELTGF

SEQ ID NO: 235 - protein, 149 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGCAENLYFQGGAAGHHHHHH

SEQ ID NO: 236 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagattggggtccatctaactggtggtccaccaactattacattaaggttcgtgccggtgacaataagtatatgcacctgaaagtgttcaacggcccggttgatgataaaaccctgtctaaagatgcggaccgtgttctgaccggttaccaggttgacaagaacaaagatgacgagctgacgggtttcgcggccgcgggtcatcaccaccaccaccattag

SEQ ID NO: 237 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagatcatggtccaatcgcatggtggtccaccaactattacattaaggttcgtgccggtgacaataagtatatgcacctgaaagtgttcaacggcccggaagataccaacaccgatggtgcactggcggaccgtgttctgaccggttaccaggttgacaagaacaaagatgacgagctgacgggtttcgcggccgcgggtcatcaccaccaccaccattag

SEQ ID NO: 238 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaaccatacggtccacgtgattgggattccaccaactattacattaaggttcgtgccggtgacaataagtatatgcacctgaaagtgttcaacggcccggaaccacagctggatacctctccaatcgcggaccgtgttctgaccggttaccaggttgacaagaacaaagatgacgagctgacgggtttcgcggccgcgggtcatcaccaccaccaccattag

SEQ ID NO: 239 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaacacctggtttccagaatctttttggtccaccaactattacattaaggttcgtgccggtgacaataagtatatgcacctgaaagtgttcaacggcccggatgataaccaggaacgtcaggaacatgcggaccgtgttctgaccggttaccaggttgacaagaacaaagatgacgagctgacgggtttcgcggccgcgggtcatcaccaccaccaccattag

SEQ ID NO: 240 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacgtgaaggtcgtcaggattgggttctgtccaccaactattacattaaggttcgtgccggtgacaataagtatatgcacctgaaagtgttcaacggcccgtgggttccatttccacatcagcagctggcggaccgtgttctgaccggttaccaggttgacaagaacaaagatgacgagctgacgggtttcgcggccgcgggtcatcaccaccaccaccattag

SEQ ID NO: 241 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacatgcatacggtccacgtgattgggattccaccaactattacattaaggttcgtgccggtgacaataagtatatgcacctgaaagtgttcaacggcccgccagcagatcatgttctggaagaagcagcggaccgtgttctgaccggttaccaggttgacaagaacaaagatgacgagctgacgggtttcgcggccgcgggtcatcaccaccaccaccattag

SEQ ID NO: 242 - DNA, 369 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagaatacggtccagaagaatggtggtccaccaactattacattaaggttcgtgccggtgacaataagtatatgcacctgaaagtgttcaacggcccgggtgattacgaacaggttctgatccatgcggaccgtgttctgaccggttaccaggttgacaagaacaaagatgacgagctgacgggtttcgcggccgcgggtcatcaccaccaccaccattag

SEQ ID NO: 243 - DNA, 371 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagctgctcatttcccggaacatttctggtccaccaactattacattaaggttcgtgccggtgacaataagtatatgcacctgaaagtgttcaacggcccgcagccggctgatatgtctgctgaattcgcggaccgtgttctgaccggttaccaggttgacaagaacaaagatgacgagctgacgggtttcctgcaggcggccgcgcaccaccaccaccaccactg

SEQ ID NO: 244 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagattggtggcaggcaaaatggccacattccaccaactattacattaaggttcgtgccggtgacaataagtatatgcacctgaaagtgttcaacggcccgtacaaagttcatcagtcttctggtggtgcggaccgtgttctgaccggttaccaggttgacaagaacaaagatgacgagctgacgggtttc

SEQ ID NO: 245 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtatctggcagtctcgttggccaggttccaccaactattacattaaggttcgtgccggtgacaataagtatatgcacctgaaagtgttcaacggcccgtttcatccaatcgcaggtcgtccatgggcggaccgtgttctgaccggttaccaggttgacaagaacaaagatgacgagctgacgggtttc

SEQ ID NO: 246 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggttactgggcagcaaaatggccaggttccaccaactattacattaaggttcgtgccggtgacaataagtatatgcacctgaaagtgttcaacggcccgtttccaaacacctcttacgatctgcaggcggaccgtgttctgaccggttaccaggttgacaagaacaaagatgacgagctgacgggtttc

SEQ ID NO: 247 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtttttacgcagatcattggccaggttccaccaactattacattaaggttcgtgccggtgacaataagtatatgcacctgaaagtgttcaacggcccgtttgcacattacaacctgaaatctggtgcggaccgtgttctgaccggttaccaggttgacaagaacaaagatgacgagctgacgggtttc

SEQ ID NO: 248 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaactggtaccagcagcgttggccaggttccaccaactattacattaaggttcgtgccggtgacaataagtatatgcacctgaaagtgttcaacggcccgtggcataactacggtgaatcttctggtgcggaccgtgttctgaccggttaccaggttgacaagaacaaagatgacgagctgacgggtttc

SEQ ID NO: 249 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtttttacgcacgtcattggccaggttccaccaactattacattaaggttcgtgccggtgacaataagtatatgcacctgaaagtgttcaacggcccgaaattttactacgcagatcatcagtgggcggaccgtgttctgaccggttaccaggttgacaagaacaaagatgacgagctgacgggtttc

SEQ ID NO: 250 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagatttttggaaggcacattggccaggttccaccaactattacattaaggttcgtgccggtgacaataagtatatgcacctgaaagtgttcaacggcccgtacacccatgcagatccacattctcaggcggaccgtgttctgaccggttaccaggttgacaagaacaaagatgacgagctgacgggtttc

SEQ ID NO: 251 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagatttttactctgttcgttggccaggttccaccaactattacattaaggttcgtgccggtgacaataagtatatgcacctgaaagtgttcaacggcccgtttggtgttccacagctgggtgcaggtgcggaccgtgttctgaccggttaccaggttgacaagaacaaagatgacgagctgacgggtttc

SEQ ID NO: 252 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcatactgggcagcaaaccatgcatctaaatccaccaactattacattaaggttcgtgccggtgacaataagtatatgcacctgaaagtgttcaacggcccgtactctggttttccatttgcaggttttgcggaccgtgttctgaccggttaccaggttgacaagaacaaagatgacgagctgacgggtttc

SEQ ID NO: 253 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaatcaaacgtctggaacattgggaatactccaccaactattacattaaggttcgtgccggtgacaataagtatatgcacctgaaagtgttcaacggcccgtggttttcttggccatacaccccactggcggaccgtgttctgaccggttaccaggttgacaagaacaaagatgacgagctgacgggtttc

SEQ ID NO: 254 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagaatgggattctccatggtctgaaaactccaccaactattacattaaggttcgtgccggtgacaataagtatatgcacctgaaagtgttcaacggcccgtactaccatccatctatccagtctaccgcggaccgtgttctgaccggttaccaggttgacaagaacaaagatgacgagctgacgggtttc

SEQ ID NO: 255 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaacataaaaacctgcgttggccattttccaccaactattacattaaggttcgtgccggtgacaataagtatatgcacctgaaagtgttcaacggcccgtttctgggttggaaagataccgttgttgcggaccgtgttctgaccggttaccaggttgacaagaacaaagatgacgagctgacgggtttc

SEQ ID NO: 256 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacgtcattttccaaaacagaccaactggtccaccaactattacattaaggttcgtgccggtgacaataagtatatgcacctgaaagtgttcaacggcccggattggtggaaatggtggtgggcaaaagcggaccgtgttctgaccggttaccaggttgacaagaacaaagatgacgagctgacgggtttc

SEQ ID NO: 257 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagtttggggtccagaataccagcatcagtccaccaactattacattaaggttcgtgccggtgacaataagtatatgcacctgaaagtgttcaacggcccgaacgcaggttggccactggttccagaagcggaccgtgttctgaccggttaccaggttgacaagaacaaagatgacgagctgacgggtttc

SEQ ID NO: 258 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaacctggaaaaacaacggtcaggatgtttccaccaactattacattaaggttcgtgccggtgacaataagtatatgcacctgaaagtgttcaacggcccgtacgcactggatccatttggtggtaaagcggaccgtgttctgaccggttaccaggttgacaagaacaaagatgacgagctgacgggtttc

SEQ ID NO: 259 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagcaacctggctgaactactacctgccatccaccaactattacattaaggttcgtgccggtgacaataagtatatgcacctgaaagtgttcaacggcccgggttacaaattttggggtgtttctgatgcggaccgtgttctgaccggttaccaggttgacaagaacaaagatgacgagctgacgggtttc

SEQ ID NO: 260 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagatcaggaatctctgtttctgaacaactccaccaactattacattaaggttcgtgccggtgacaataagtatatgcacctgaaagtgttcaacggcccgcagggtaaacagtacatcctgctgcgtgcggaccgtgttctgaccggttaccaggttgacaagaacaaagatgacgagctgacgggtttc

SEQ ID NO: 261 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtttttacgcacagcattggccagattccaccaactattacattaaggttcgtgccggtgacaataagtatatgcacctgaaagtgttcaacggcccgtacaaacgtcattctgcacatgattacgcggaccgtgttctgaccggttaccaggttgacaagaacaaagatgacgagctgacgggtttc

SEQ ID NO: 262 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtcattacgcacgttactggccaggttccaccaactattacattaaggttcgtgccggtgacaataagtatatgcacctgaaagtgttcaacggcccgtgggcacagaaatctaaagttcatcaggcggaccgtgttctgaccggttaccaggttgacaagaacaaagatgacgagctgacgggtttc

SEQ ID NO: 263 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtttttgggcaagtaaatggccaggttccaccaactattacattaaggttcgtgccggtgacaataagtatatgcacctgaaagtgttcaacggcccgtttaccgcagtttctaaaaaagatgcagcggaccgtgttctgaccggttaccaggttgacaagaacaaagatgacgagctgacgggtttc

SEQ ID NO: 264 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtttttggcagcgtaaatggccaaactccaccaactattacattaaggttcgtgccggtgacaataagtatatgcacctgaaagtgttcaacggcccgtggggtgataaagaaaacatctggtttgcggaccgtgttctgaccggttaccaggttgacaagaacaaagatgacgagctgacgggtttc

SEQ ID NO: 265 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagtttggccagcagataacgatctgaaatccaccaactattacattaaggttcgtgccggtgacaataagtatatgcacctgaaagtgttcaacggcccgtggtctggtcatccatgggttcagaaagcggaccgtgttctgaccggttaccaggttgacaagaacaaagatgacgagctgacgggtttc

SEQ ID NO: 266 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacattgggcatggacctctccaggttactccaccaactattacattaaggttcgtgccggtgacaataagtatatgcacctgaaagtgttcaacggcccgtacgcagattacccactgtctccaaaagcggaccgtgttctgaccggttaccaggttgacaagaacaaagatgacgagctgacgggtttc

SEQ ID NO: 267 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaacttttttcagcgtcgttggccaggttccaccaactattacattaaggttcgtgccggtgacaataagtatatgcacctgaaagtgttcaacggcccgtggaaatttcgtaacaccgatcgtggtgcggaccgtgttctgaccggttaccaggttgacaagaacaaagatgacgagctgacgggtttc

SEQ ID NO: 268 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacatcattctcatcgtctgaaaggtcagtccaccaactattacattaaggttcgtgccggtgacaataagtatatgcacctgaaagtgttcaacggcccgcagaccgttgcaacccattaccattacgcggaccgtgttctgaccggttaccaggttgacaagaacaaagatgacgagctgacgggtttc

SEQ ID NO: 269 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcataccagaacaccatctttctgtctatctccaccaactattacattaaggttcgtgccggtgacaataagtatatgcacctgaaagtgttcaacggcccgtggcatgcaaaacatctgctgtctcatgcggaccgtgttctgaccggttaccaggttgacaagaacaaagatgacgagctgacgggtttc

SEQ ID NO: 270 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcatttcaggatcagtttacctggtctcagtccaccaactattacattaaggttcgtgccggtgacaataagtatatgcacctgaaagtgttcaacggcccgtctggtatcaaaaaagcagattctgttgcggaccgtgttctgaccggttaccaggttgacaagaacaaagatgacgagctgacgggtttc

SEQ ID NO: 271 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaggtgaaccacattggccatggcaggcatccaccaactattacattaaggttcgtgccggtgacaataagtatatgcacctgaaagtgttcaacggcccgaaagcaaatttgataaacgtgaaatctgcggaccgtgttctgaccggttaccaggttgacaagaacaaagatgacgagctgacgggtttc

SEQ ID NO: 272 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagcagatccacgtcatccatgggttgaatccaccaactattacattaaggttcgtgccggtgacaataagtatatgcacctgaaagtgttcaacggcccgtggaaatctcatgttgaagttcgttctgcggaccgtgttctgaccggttaccaggttgacaagaacaaagatgacgagctgacgggtttc

SEQ ID NO: 273 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcatttcataaacgttttcagtctcagggttccaccaactattacattaaggttcgtgccggtgacaataagtatatgcacctgaaagtgttcaacggcccgtgggttacccagaaatacatcatccaggcggaccgtgttctgaccggttaccaggttgacaagaacaaagatgacgagctgacgggtttc

SEQ ID NO: 274 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagaatggtggcagaaccgttggccaaactccaccaactattacattaaggttcgtgccggtgacaataagtatatgcacctgaaagtgttcaacggcccgtgggaacatgcaaaagattggccaaccgcggaccgtgttctgaccggttaccaggttgacaagaacaaagatgacgagctgacgggtttc

SEQ ID NO: 275 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagaatggtaccagacccgttggccaggttccaccaactattacattaaggttcgtgccggtgacaataagtatatgcacctgaaagtgttcaacggcccgtttcattctaaagttctggataaagcagcggaccgtgttctgaccggttaccaggttgacaagaacaaagatgacgagctgacgggtttc

SEQ ID NO: 276 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagaattttggcagcgtcattggccaggttccaccaactattacattaaggttcgtgccggtgacaataagtatatgcacctgaaagtgttcaacggcccgtacggtgcacagaaacaggcagtttgggcggaccgtgttctgaccggttaccaggttgacaagaacaaagatgacgagctgacgggtttc

SEQ ID NO: 277 - protein, 254 aa, Artificial Sequence
  Synthetic
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 278 - protein, 255 aa, Artificial Sequence
  Synthetic
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 279 - protein, 254 aa, Artificial Sequence
  Synthetic
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 280 - protein, 255 aa, Artificial Sequence
  Synthetic
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 281 - protein, 254 aa, Artificial Sequence
  Synthetic
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 282 - protein, 255 aa, Artificial Sequence
  Synthetic
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 283 - protein, 398 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 284 - protein, 398 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 285 - protein, 398 aa, Artificial Sequence
  Synthetic
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 286 - protein, 399 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFC

SEQ ID NO: 287 - protein, 398 aa, Artificial Sequence
  Synthetic
MIPRGLSEAKPATPEIQEIVDKVKPQLEEKTNETYGKLEAVQYKTQVLAFALPEFEYMSTNYYIKVRAGDNKYMHLKVFNGPPMIRRKNEVADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAFALPEFEYMSTNYYIKVRAGDNKYMHLKVFNGPPMIRRKNEVADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 288 - protein, 398 aa, Artificial Sequence
  Synthetic
MIPRGLSEAKPATPEIQEIVDKVKPQLEEKTNETYGKLEAVQYKTQVLAGGGGGGGGGSTNYYIKVRAGDNKYMHLKVFNGPGGGGGGGGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAGGGGGGGGGSTNYYIKVRAGDNKYMHLKVFNGPGGGGGGGGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 289 - protein, 112 aa, Artificial Sequence; identical to SEQ ID NO: 224
  Synthetic

SEQ ID NO: 290 - protein, 398 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 291 - protein, 398 aa, Artificial Sequence
  Synthetic
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 292 - protein, 6 aa, Artificial Sequence
  Synthetic
HHHHHH

SEQ ID NO: 293 - protein, 35 aa, Artificial Sequence
  Synthetic
  MISC_FEATURE (11)..(35): May be absent
GGGGSGGGGSGGGGSGGGGSGGGGSGGGGSGGGGS

SEQ ID NO: 294 - protein, 36 aa, Artificial Sequence
  Synthetic
AEAAAKEAAAKEAAAKEAAAKEAAAKEAAAKEAAAK

SEQ ID NO: 295 - protein, 10 aa, Artificial Sequence
  Synthetic
EQKLISEEDL

SEQ ID NO: 296 - protein, 7 aa, Artificial Sequence
  Synthetic
  SITE (7)..(7): X may be G or S
ENLYFQX

SEQ ID NO: 297 - DNA, 336 bp, Artificial Sequence
  Synthetic
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagaacatggtccagattcttggtggtccaccaactattacattaaggttcgtgccggtgacaataagtatatgcacctgaaagtgttcaacggcccgcaggaaaaaaaccagtgggttgaagaagcggaccgtgttctgaccggttaccaggttgacaagaacaaagatgacgagctgacgggtttc

SEQ ID NO: 298 - protein, 9 aa, Artificial Sequence
  Synthetic
GGGGGGGGG

SEQ ID NO: 299 - protein, 7 aa, Artificial Sequence
  Synthetic
GGGGGGG

SEQ ID NO: 300 - protein, 18 aa, Artificial Sequence
  Synthetic
KESGSVSSEQLAQFRSLD

SEQ ID NO: 301 - protein, 14 aa, Artificial Sequence
  Synthetic
EGKSSGSGSESKST

SEQ ID NO: 302 - protein, 12 aa, Artificial Sequence
  Synthetic
GSAGSAAGSGEF

SEQ ID NO: 303 - protein, 35 aa, Artificial Sequence
  Synthetic
  MISC_FEATURE (11)..(35): May be absent
EAAAKEAAAKEAAAKEAAAKEAAAKEAAAKEAAAK

SEQ ID NO: 304 - protein, 56 aa, Artificial Sequence
  Synthetic
AEAAAKEAAAKEAAAKEAAAKEAAAKALEAEAAAKEAAAKEAAAKEAAAKEAAAKA

SEQ ID NO: 305 - protein, 5 aa, Artificial Sequence
  Synthetic
PAPAP

SEQ ID NO: 306 - protein, 12 aa, Artificial Sequence
  Synthetic
AEAAAKEAAAKA

SEQ ID NO: 307 - protein, 70 aa, Artificial Sequence
  Synthetic
  MISC_FEATURE (5)..(70): May be absent
APAPAPAPAPAPAPAPAPAPAPAPAPAPAPAPAPAPAPAPAPAPAPAPAPAPAPAPAPAPAPAPAPAPAP

SEQ ID NO: 308 - protein, 9 aa, Artificial Sequence
  Synthetic
  SITE (1)..(1): Xaa may be K, R, or H
  SITE (2)..(2): Xaa may be A, P, I,  Q, T, D, E, K, R, or H
  SITE (3)..(3): Xaa may be F,Y, W, K, R, or H
  SITE (6)..(6): Xaa may be A, P, I, Q, T, D, E, K, R, or H
  SITE (7)..(7): Xaa may be S, T, N, Q, D, E, R, or H
  SITE (9)..(9): Xaa may be F, Y, W, D, or E
XXXGPXXWX

SEQ ID NO: 309 - protein, 9 aa, Artificial Sequence
  Synthetic
  SITE (1)..(1): Xaa may be D, G, N, or V
  SITE (2)..(2): Xaa may be W, Y, H, or F
  SITE (3)..(3): Xaa may be W, Y, G, or F
  SITE (4)..(4): Xaa may be Q, A, or P
  SITE (5)..(5): Xaa may be A, Q, E, R, or S
  SITE (6)..(6): Xaa may be K, R, or Y
  SITE (7)..(7): Xaa may be W or Q
  SITE (8)..(8): Xaa may be P or H
  SITE (9)..(9): Xaa may be H, G, or Q
XXXXXXXXX

SEQ ID NO: 310 - protein, 9 aa, Artificial Sequence
  Synthetic
  SITE (1)..(1): Xaa may be K, R, H, S, T, N, or Q
  SITE (2)..(2): Xaa may be A, P, I,  Q, T, D, E, K, R, or H
  SITE (3)..(3): Xaa may be F,Y, W, K, R, or H
  SITE (6)..(6): Xaa may be A, P, I, Q, T, D, E, K, R, or H
  SITE (7)..(7): Xaa may be S, T, N, Q, D, E, R, or H
XXXFPXXFW

SEQ ID NO: 311 - protein, 9 aa, Artificial Sequence
  Synthetic
  SITE (1)..(1): Xaa may be G, A, V, P, W, Q, S, D, or E
  SITE (2)..(2): Xaa may be D, E, K, R, H, Q, S, T, N, A, V, P, G, Y, or F
  SITE (3)..(3): Xaa may be Q, S, T, N, D, E, R, K, G, L, P, or Y
  SITE (4)..(4): Xaa may be D, E, R, H, K, S, Q, N, A, L, Y, W, P, or G
  SITE (5)..(5): Xaa may be D, E, S, T, Q, R, K, H, V, I, Y, or G
  SITE (6)..(6): Xaa may be D, E, S, T, Q, N, K, R, V, L, I, W, Y, F, or G
  SITE (7)..(7): Xaa may be S, T, Q, N, V, I, L, G, P, D, E, H, R, W, Y, or F
  SITE (8)..(8): Xaa may be A, I, W, P, D, E, R, K, H, S, T, Q, or N
  SITE (9)..(9): Xaa may be H, R, K, D, S, T, Q, N, A, V, L, G, or F
XXXXXXXXX

SEQ ID NO: 312 - protein, 9 aa, Artificial Sequence
  Synthetic
  SITE (1)..(1): Xaa may be Y, F, W, or N
  SITE (2)..(2): Xaa may be K, P, H, A, or T
  SITE (3)..(3): Xaa may be V, N, G, Q, A, or F
  SITE (4)..(4): Xaa may be H, T, Y, W, K, V,  or R
  SITE (5)..(5): Xaa may be Q, S, G, P, or N
  SITE (6)..(6): Xaa may be S, Y, E, L, K, or T
  SITE (7)..(7): Xaa may be S, D, V, or K
  SITE (8)..(8): Xaa may be G, L, S, P, H, D, or R
  SITE (9)..(9): Xaa may be G, Q, E, or A
XXXXXXXXX

SEQ ID NO: 313 - protein, 4 aa, Artificial Sequence
  Synthetic
GGSG

SEQ ID NO: 314 - protein, 4 aa, Artificial Sequence
  Synthetic
GSGS

SEQ ID NO: 315 - protein, 4 aa, Artificial Sequence
  Synthetic
GGGS

SEQ ID NO: 316 - protein, 7 aa, Artificial Sequence
  Synthetic
GGGSGGG

SEQ ID NO: 317 - protein, 8 aa, Artificial Sequence
  Synthetic
ESGGGGVT

SEQ ID NO: 318 - protein, 9 aa, Artificial Sequence
  Synthetic
LESGGGGVT

SEQ ID NO: 319 - protein, 6 aa, Artificial Sequence
  Synthetic
GRAQVT

SEQ ID NO: 320 - protein, 6 aa, Artificial Sequence
  Synthetic
WRAQVT

SEQ ID NO: 321 - protein, 8 aa, Artificial Sequence
  Synthetic
ARGRAQVT